(19) United States
(12) Patent Application Publication
(10) Pub. No.: US 2010/0234292 A1
(43) Pub. Date: Sep. 16, 2010

(54) METHODS OF ASSESSING A PROPENSITY OF CLINICAL OUTCOME FOR A FEMALE MAMMAL SUFFERING FROM BREAST CANCER

(75) Inventors: Francois Bertucci, Carnoux-en-Provence (FR); Daniel Birnbaum, Marseille (FR); Patrice Viens, Marseille (FR); Vincent Fert, Marseille (FR); Fabienne Hermitte, La Ciotat (FR); Stephane Debono, Marseille (FR); Stephane Deraco, Saint-Orens-de-Gameville (FR); Nathalie Borie, Clermont-Ferrand (FR); Fanny Piette, Limelette (BE)

Correspondence Address: ARENT FOX LLP, 1050 CONNECTICUT AVENUE, N.W., SUITE 400, WASHINGTON, DC, 20036, US

(73) Assignees: IPSOGEN, Marseille (FR); INSERM - INSTITUT NATIONAL DE LA SANTE ET DE LA RE CHECHE MEDICALE, Paris Cedex 13 (FR); INSTITUT PAOLI-CALMETTES, Marseille (FR)

(21) Appl. No.: 12/596,143

(22) PCT Filed: Apr. 16, 2008
(86) PCT No.: PCT/IB08/02334

§ 371 Date: May 21, 2010

Related U.S. Application Data
(60) Provisional application No. 60/923,690, filed on Apr. 16, 2007.

(51) Int. Cl. C12Q 1/68; A61K 38/16; A61K 38/22; A61K 38/02; A61P 35/00

(52) U.S. Cl. 514/12; 435/6; 514/2

(57) ABSTRACT

The present invention relates to a method of assessing a propensity of clinical outcome for a female mammal suffering from breast cancer in view of the expression of specific nucleic acid sequences in a biological sample.

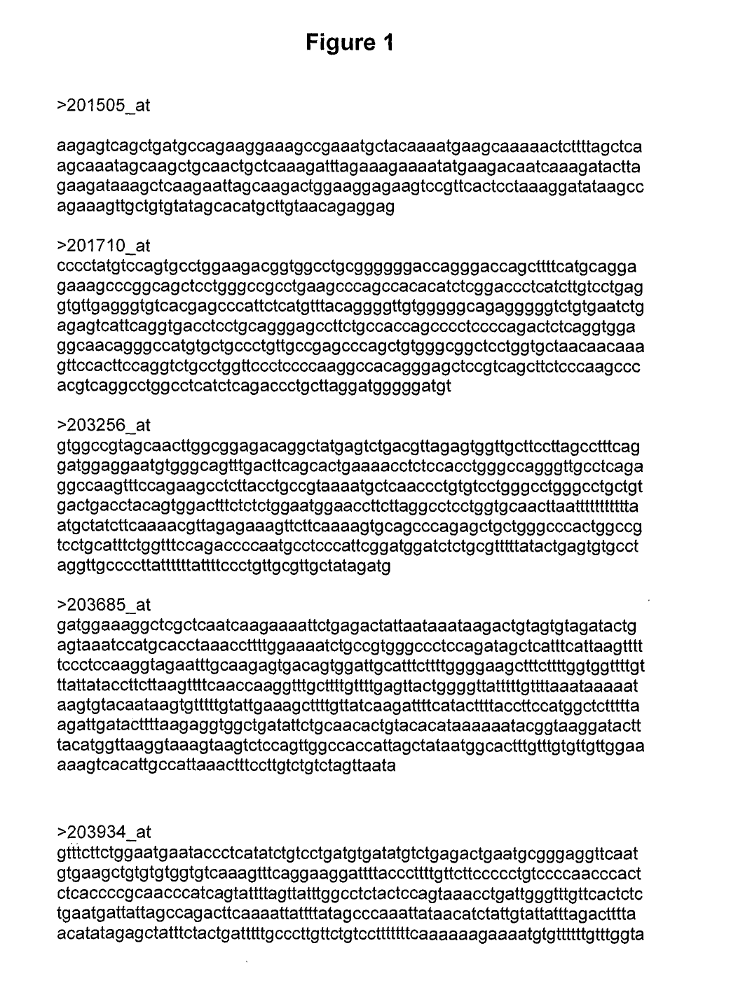

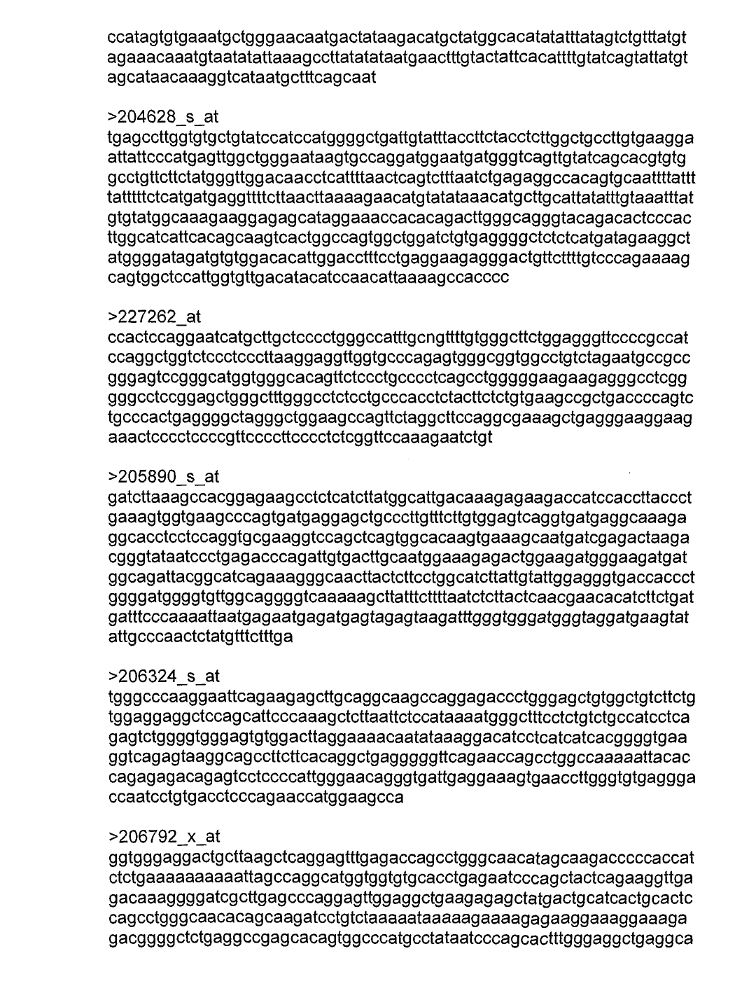

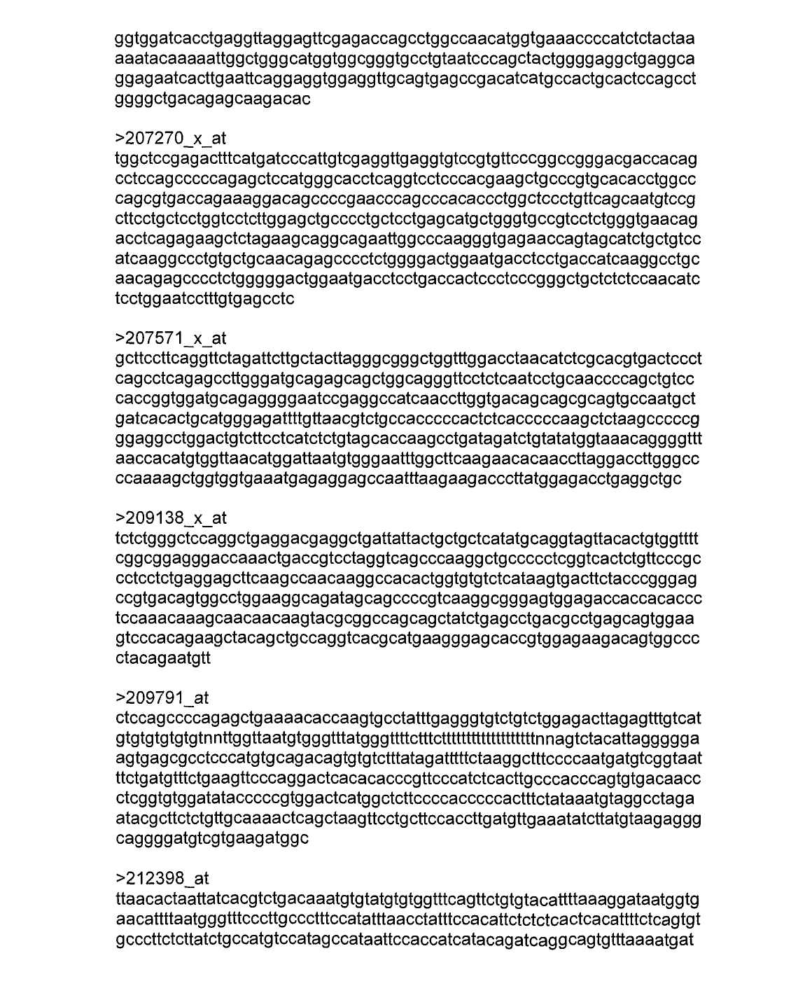

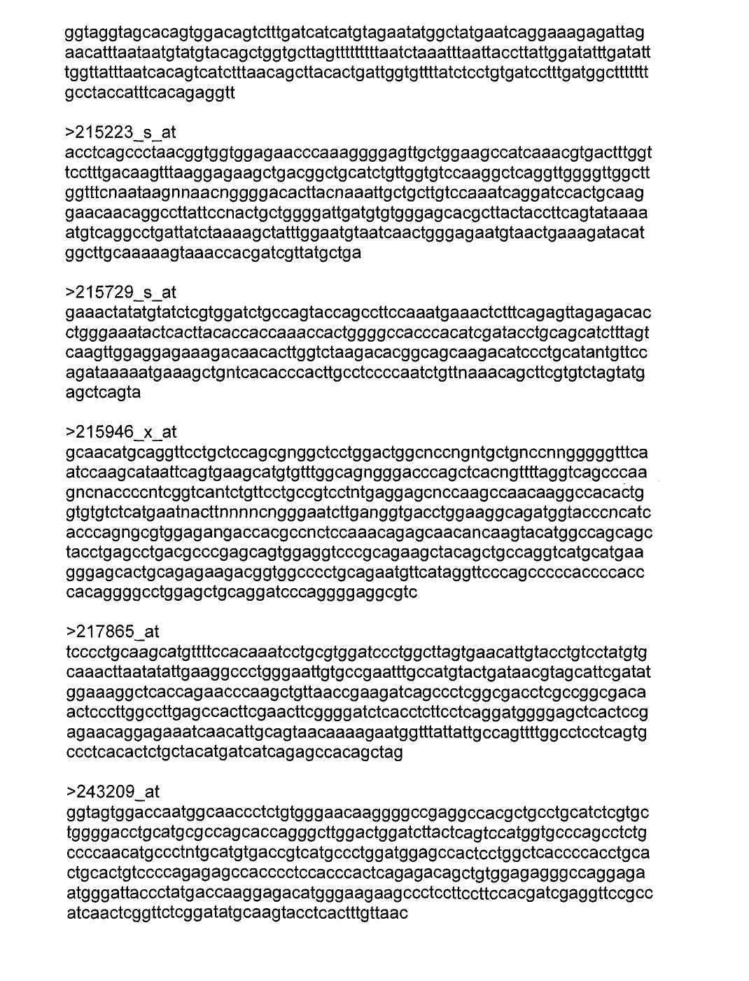

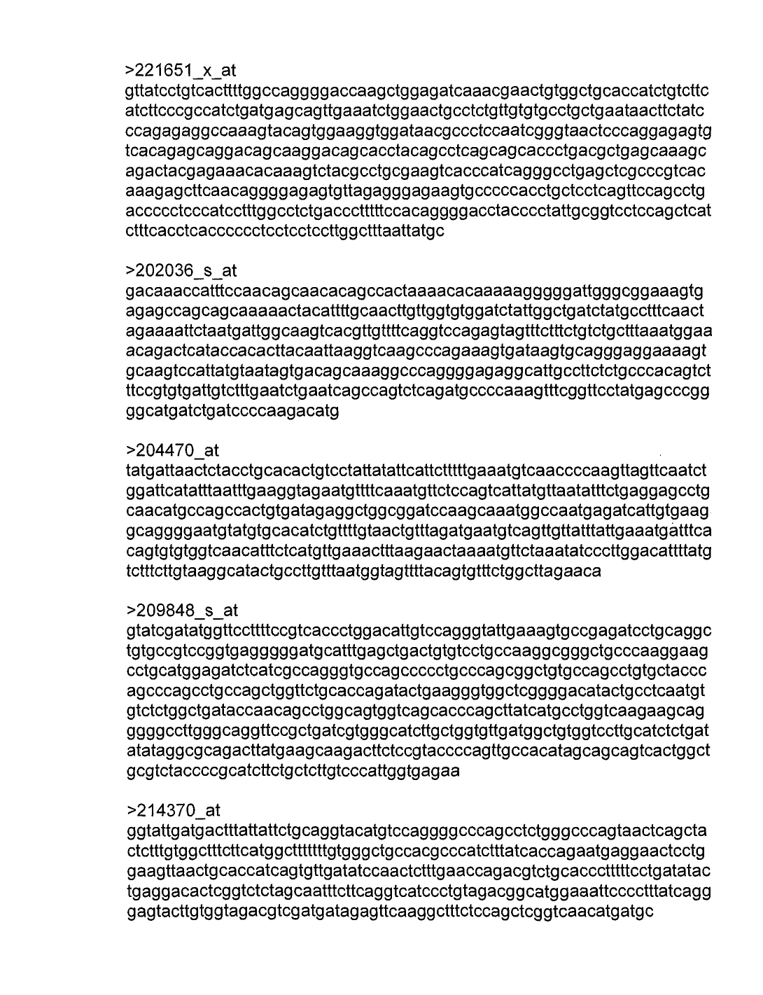

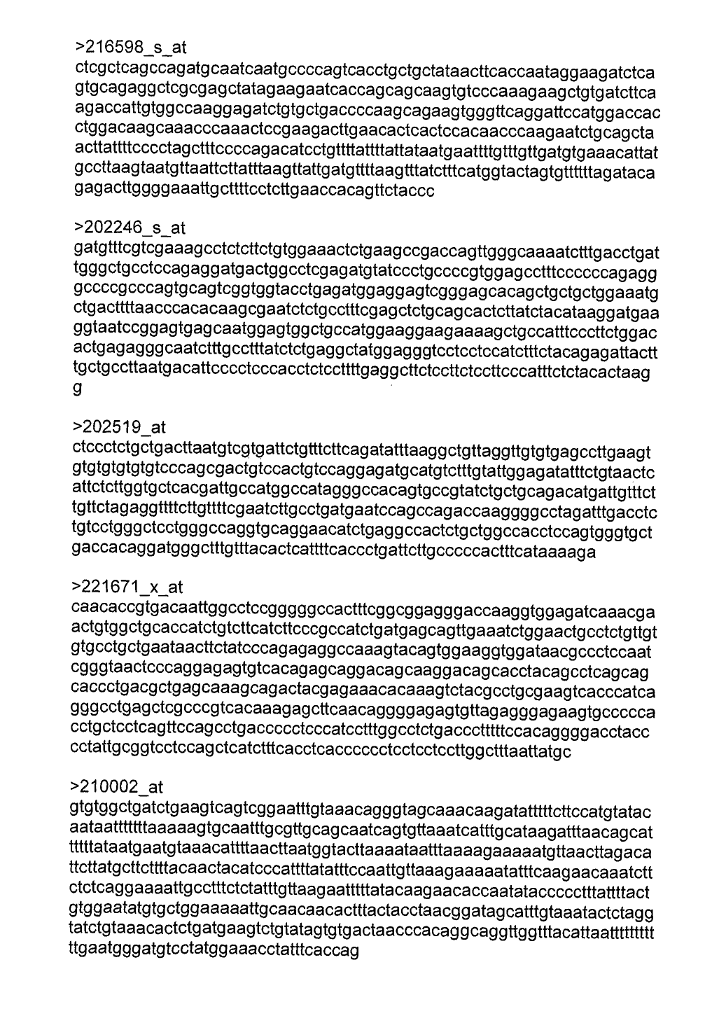

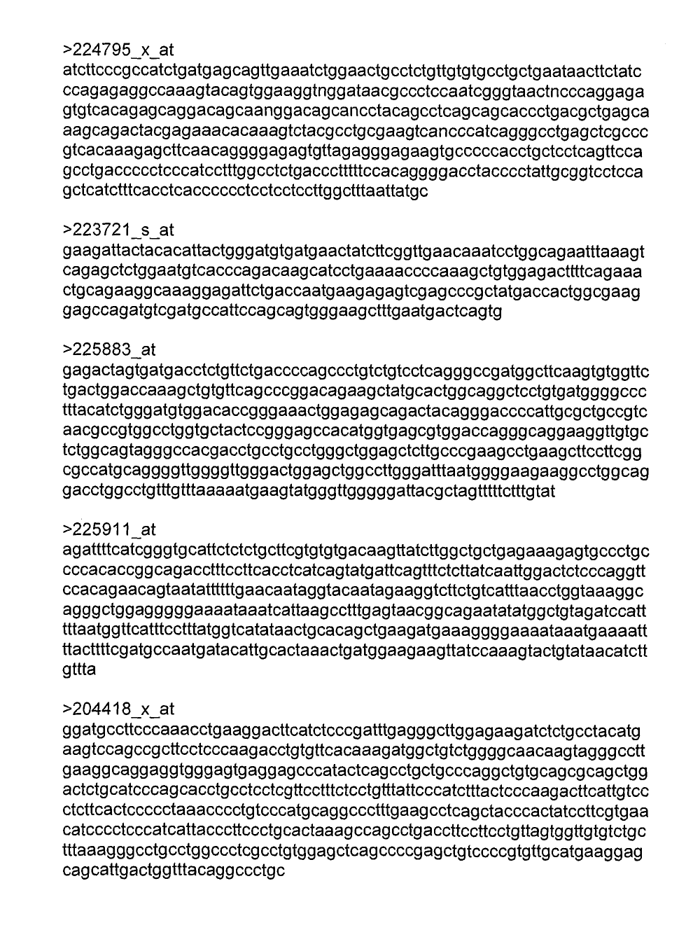

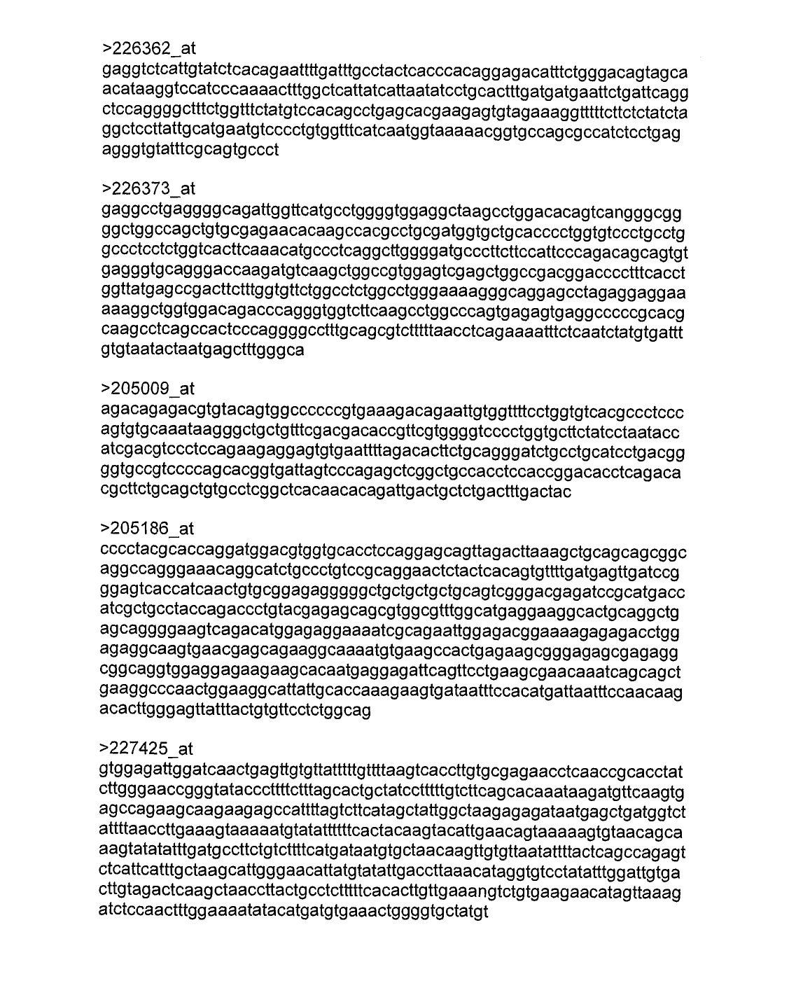

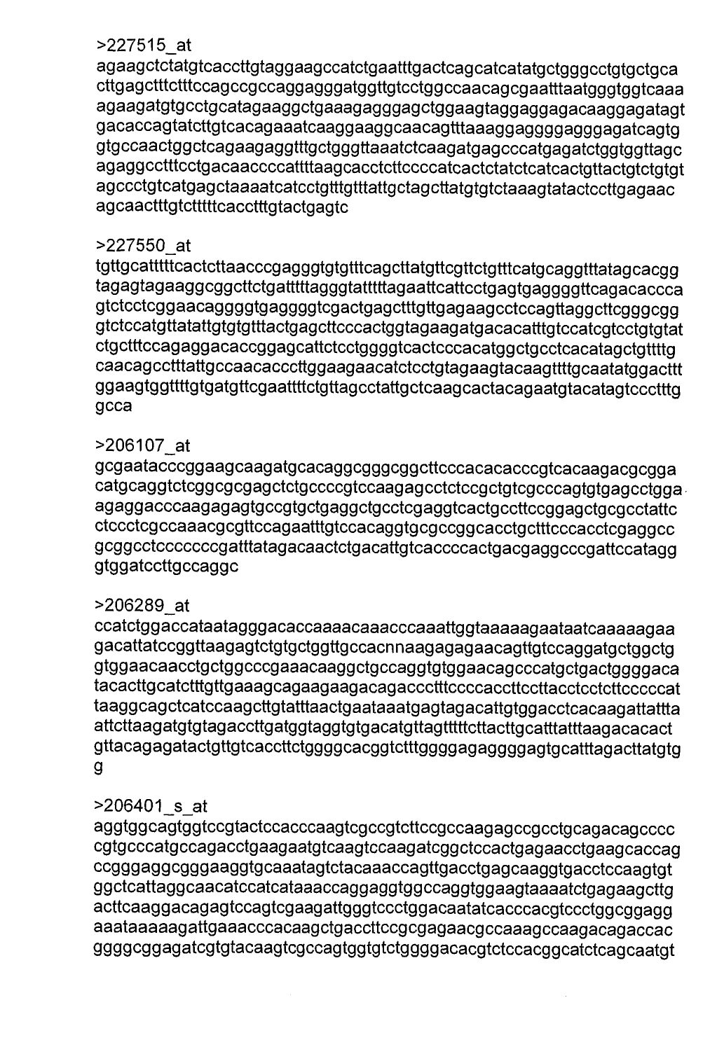

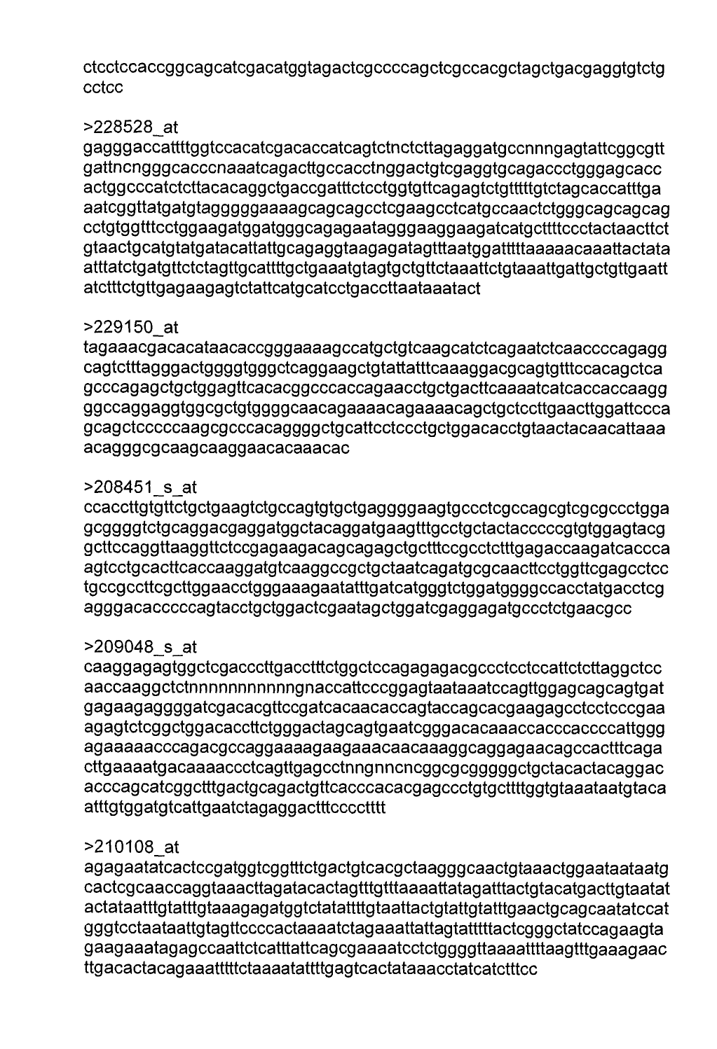

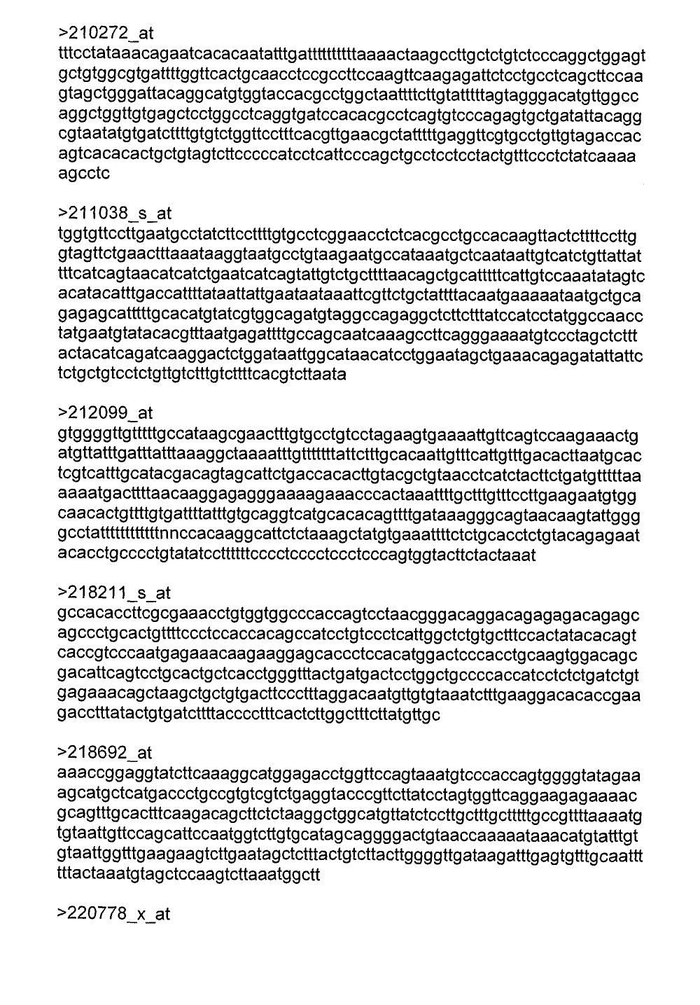

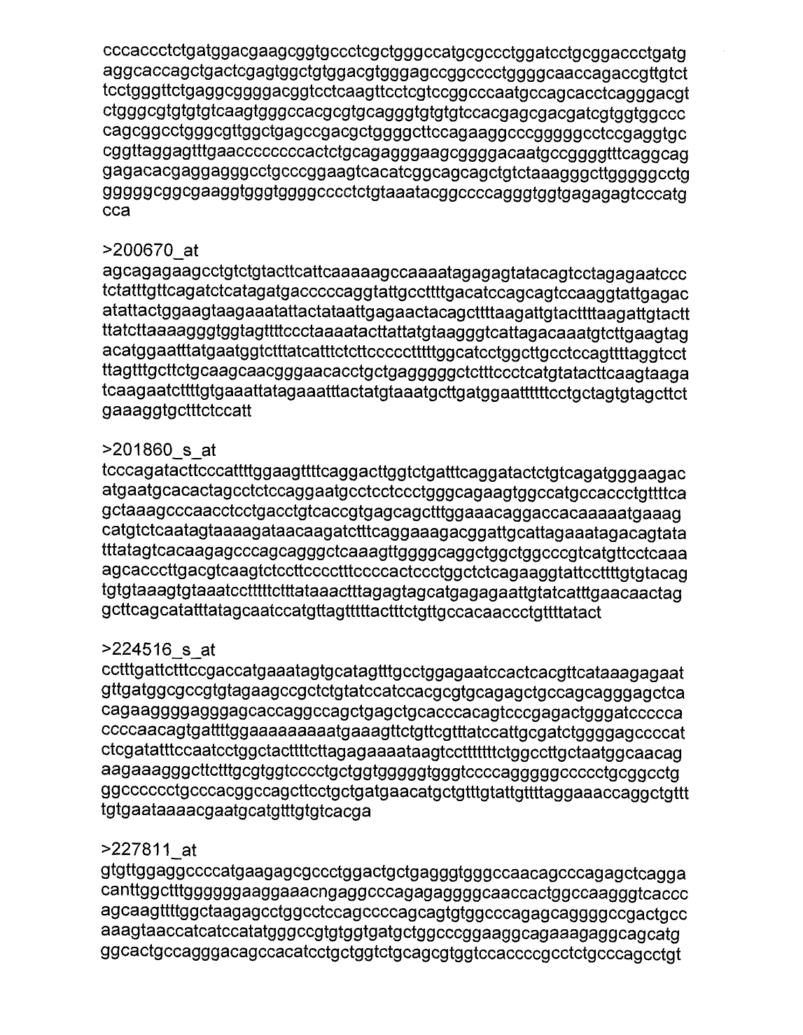

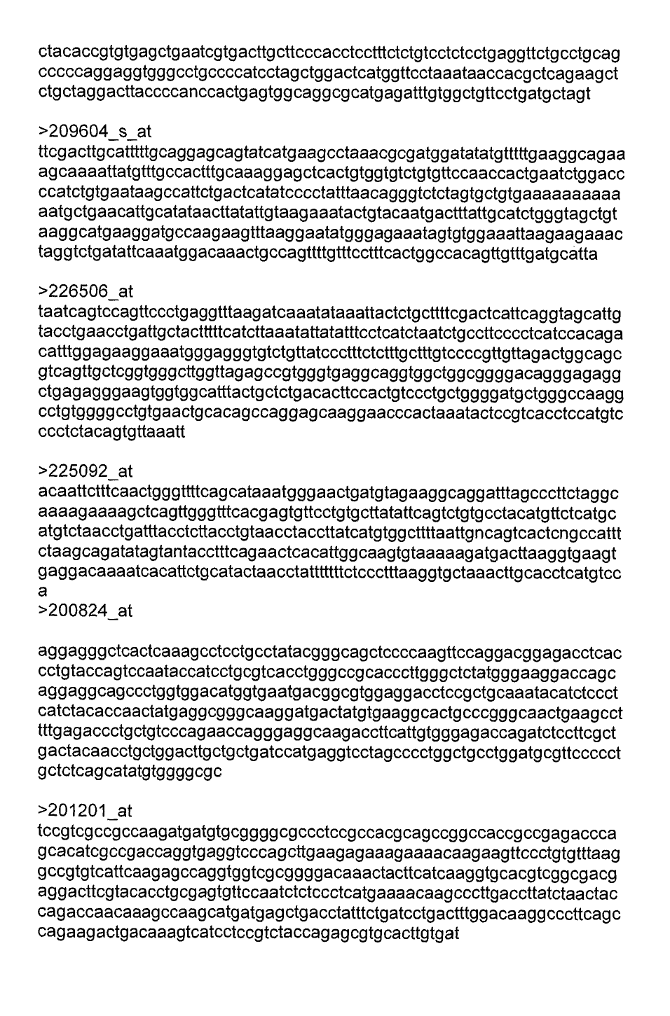

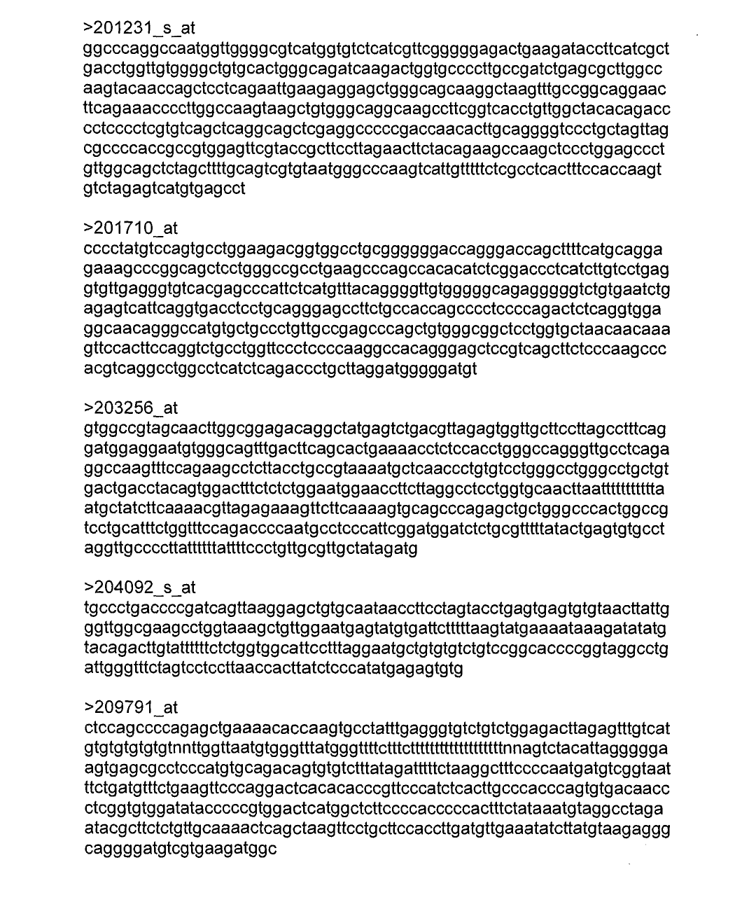

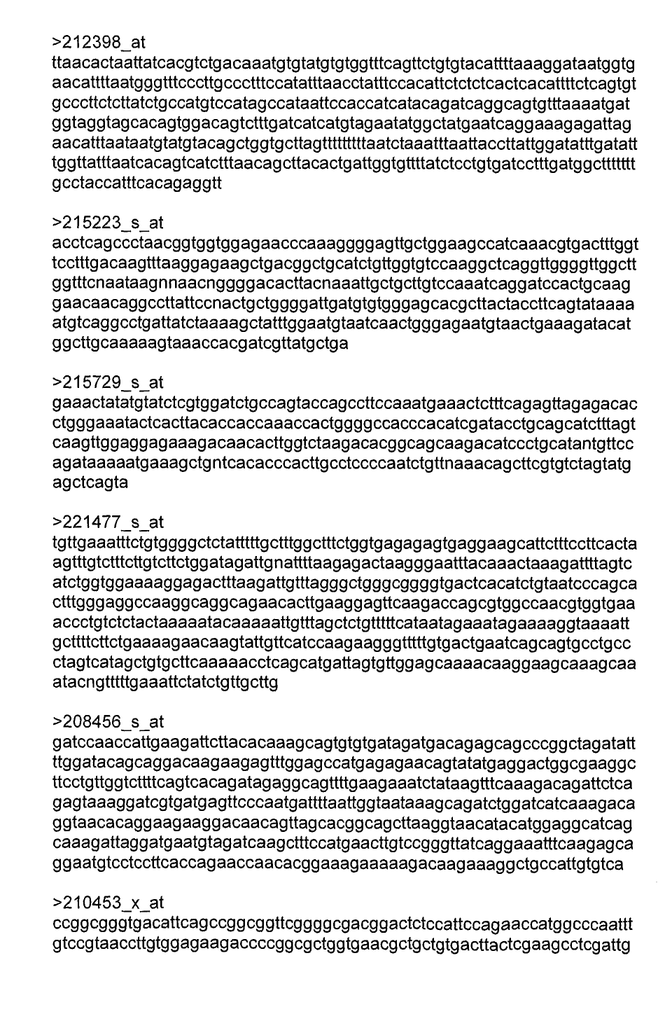

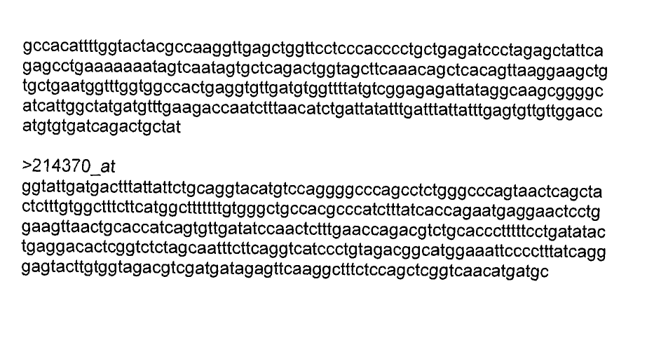

### FIELD OF THE INVENTION

The present invention relates to methods of assessing a propensity of the clinical outcome of a female mammal suffering from breast cancer, preferably after said female mammal has been treated with chemotherapy, for example anthracycline-based chemotherapy.

### BACKGROUND

Breast cancer is the most common nonskin malignancy in women and the second leading cause of female cancer mortality (FEAR et al., IEEE Potentials, vol. 22 (1), p: 12-18, 2003).

Worldwide, breast cancer is the most common cancer in women. It is estimated than in the year 2000, there were 350.000 new breast cancer cases in Europe, while the number of deaths from breast cancer was estimated at 130.000. Breast cancer is responsible for 26.5% of all new cancer cases among women in Europe, and 17.5% of cancer deaths. The highest incidence rates for the year 2000 were in Western Europe, with France in third position (42.000 new cases and 12.000 deaths). Despite these high rates of incidence and mortality, the survival of women diagnosed with breast cancer increased in Europe and in France since the end of the 1970s. This improvement is probably in relation with early diagnosis and screening programs and with adjuvant systemic therapy.

Adjuvant chemotherapy (CT) for breast cancer has undergone major changes over the past two decades. Results from the published update of the overview analysis by the Early Breast Cancer Trialists' Collaborative group indicated that administration of adjuvant CT significantly reduced the risk of recurrence by 23.5% and the risk of death by 15.3%. According to the same overview, the 10-year recurrence-free survival for node-positive patients treated with adjuvant CT was 47.6% for patients younger than 50 years and 43.6% for those 50 to 69 years of age. The 10-year overall survival (OS) was 53.8% and 48.6% respectively. This overview analysis also demonstrated that, as compared with standard combination of cyclophosphamide, methotrexate and 5FU (CMF), regimens that contained anthracyclins reduced the annual risk of recurrence of breast cancer by 12% and the annual risk of death by 11%. Such regimens are significantly (2p=0.0001 for recurrence, 2p<0.00001 for breast cancer mortality) more effective than CMF.

The most commonly used anthracycline-based adjuvant CT regimen in USA consists of four cycles of doxorubicin plus cyclophosphamide (AC) administrated every 21 days. Six cycles of FAC (cyclophosphamide, doxorubicin, and fluorouracil) every 3 weeks were also accepted as appropriate adjuvant regimen. Since epirubicin is less cardiotoxic than doxorubicin at an equimolar dose (recommended cumulative doses of doxorubicin and epirubicin are 550 mg/m² and 1.000 mg/m², respectively), several groups introduced epirubicin. A National Cancer Institute of Canada study showed that six cycles of cyclophosphamide, epirubicin, fluorouracil (CEF) were superior to six cycles of CMF. The Groupe Français d'Etudes Adjuvantes (GFEA; The French Adjuvant Trial Group) has studied epirubicin in the treatment of breast cancer for several years. The FEC regimen (fluorouracil, epirubicin, cyclophosphamide) has been evaluated in the trial setting lymph node-positive patients. Six cycles of adjuvant FEC 50 (epirubicin 50 mg/m²) are better than 3 cycles. Subsequently a trial in patients less than 65 years of age, with node-positive operable breast cancer, compared FEC 50 versus FEC 100 (epirubicin 100 mg/m²). Six cycles of FEC 100 was associated with improved relapse rates and better survival. Thus, 6 cycles of FEC every three weeks were generally accepted a few years ago in France as appropriate and “standard” adjuvant regimens for early breast cancer.

Recently, taxanes have emerged as potent agents for the adjuvant treatment of breast cancer. Studies involving more than 20.000 patients have been reported or are ongoing. Recent published adjuvant trials with taxanes (paclitaxel, docetaxel) in node-positive breast cancer have demonstrated an additional benefit (as compared with regimen without taxanes), ranging from 2 to 7% in absolute difference in disease-free survival (DFS) or overall survival (OS) at 5 years. Two trials showed the benefit of incorporating sequentially 4 courses of paclitaxel after 4 cycles of AC: CALGB 9344 and NSABP B-28. Two trials showed the benefit of incorporating docetaxel: BCIRG 01 study, which compared the FAC regimen (6 cycles) to the TAC regimen (docetaxel, doxorubicin, and fluorouracil, 6 cycles), and PACS 01 study. The PACS 01 study (1.999 patients included) was promoted by the French Federation of Anti-Cancer Centers (FNCLCC). It compared the FEC 100 regimen (6 cycles) to a sequential regimen, 3 cycles of FEC100 followed by 3 cycles of docetaxel administered at the dose of 100 mg/m² every 3 weeks in node-positive patients. At a median follow-up of 60 months, adjuvant CT with 3 cycles of FEC100 followed by 3 cycles of docetaxel improved recurrence-free survival (reduction in the hazard rate of recurrence, 17%, p=0.04) and OS (reduction in the hazard rate of death, 23% p=0.005) (13). The 5-year DFS are 78.3% (3 FEC100-3 docetaxel arm) vs 73.2% (6 FEC100 arm) and the 5-year OS are 90.7 vs 86.7 respectively. In comparison with the BCIRG study, the incidence of febrile neutropenia, infection and cardiac dysfunction is very low especially in the sequential arm. As a consequence of these trials, the combination of anthracyclin and taxane has become the new standard of adjuvant CT for node-positive breast cancer. Several other trials promoted by the FNCLCC (PACS) investigated the optimal scheme of combination eprubicin-docetaxel: the PACS 04 study compared the FEC 100 regimen (6 cycles) to the combination epirubicin 75 mg/m²+docetaxel 75 mg/m² every 3 weeks in node-positive patients. Follow-up is ongoing with 3.015 patients included (end of inclusions in August 2004). The PACS 06 compared FEC 100×3 cycles every 2 weeks followed by docetaxel 100 mg/m^{2×3} cycles every 2 weeks, in association with G-CSF, with either a 2-week or a 4-week interval between FEC and docetaxel. The primary endpoint was to define the rate of patients with any toxicity requiring dose reduction or treatment delay by more than one week over the 6 courses. As May 2005, the recruitment was stopped after 74 inclusions with the following conclusion, FEC 100×3 cycles every 2 weeks followed by docetaxel 100 mg/m^{2×3} cycles every 2 weeks, with a 2-week interval between FEC and docetaxel is not feasible due to an excess of skin/hand-foot syndrome severe toxicities.

Currently, adjuvant CT in early breast cancer is indicated according classical prognostic factors such the axillary lymph node status, the pathological size and grading of tumour, the hormonal receptor expression, and age of patients. These factors remain insufficient for reflecting the whole heterogeneity of disease, and none of them has been validated for selecting the optimal regimen of CT, resulting in the delivery of a combination of anthracyclin-taxane to all node-positive patients. However, recent studies have shown that in sub-groups of patients the addition of taxanes did not provide benefit as compared to FAC or FEC and that these classical regimens without taxanes might provide long survival in certain patients. Altogether with the potential toxicity and cost of the combination of anthracyclin-taxane, as well as the ongoing introduction/development of new drugs in adjuvant regimens (CT such as capecitabine, targeted therapy such as trastuzumab, hormone therapy such as anti-aromatases, diphosphonates), these data call for the identification of parameters predictive of clinical outcome (prognostic and/or predictive of response to CT) after given regimen of adjuvant CT.

A lot of research, mainly retrospective, has been performed to find predictive biological factors of adjuvant CT effectiveness, but, presently, there is still no individual admitted factor. The current prognostic factors evaluate only poorly the heterogeneous clinical behavior of disease. In consequence, many N− patients are subjected to unnecessary anthracycline-based adjuvant CT, and all N+ patients receive regimens based on anthracyclines and taxanes (Piccart et al. The Breast 14:439-445, 2005). However, taxanes are not yet universally accepted as standard treatment (Colozza et al. Oncologist 11:111-125, 2006). Recent randomized studies (Buzdar et al. Clin Cancer Res 8:1073-1079, 2002; Henderson et al. J Clin Oncol 21:976-983, 2003; Mamounas et al. J Clin Oncol 23:3686-3696, 2005; Martin et al. N Engl J Med 352:2302-2313, 2005; Roche et al. J Clin Oncol 24:5664-5671, 2006) have shown that the addition of taxanes provides a significant but small benefit (3 to 7%) in 5-year survival. This suggests that a majority of patients do not benefit from the anthracycline-taxane combination. The availability of new drugs in adjuvant setting and the heterogeneity of breast cancer render necessary the tailoring of treatment without systematically associating all drugs. This challenge supposes to better assess the metastatic risk after CT. No biological factor predictive of anthracycline-based adjuvant CT efficacy (Hayes, The Breast 14:493-499, 2005) has yet been validated and introduced in routine use.

A predictive factor will be of a tremendous interest to select patients who benefit or who do not benefit from a specific regimen of adjuvant CT. Breast cancer is a complex genetic disease characterized by the accumulation of multiple molecular alterations. Pathological and clinical factors are insufficient to capture the complex cascade of events which drive the heterogeneous clinical behaviour of tumours.

High-throughput molecular technologies provide novel tools to tackle this complexity. In particular, DNA microarrays allow the simultaneous and quantitative analysis of the mRNA expression levels of thousands of genes in a single assay. The first research results are promising; comprehensive gene expression profiles of breast tumours are revealing new sub-groups of tumour in groups a priori identical, but with different outcome.

Several retrospective studies confirm the prognostic potential of DNA microarrays in breast cancer (Bertucci et al. Omics 10:429-443, 2006). Most studies focused on survival without any adjuvant systemic therapy (van de Vijver et al. N Engl J Med 347:1999-2009, 2002; van 't Veer et al. Nature 415:530-536, 2002; Wang et al. Lancet 365:671-679, 2005; Foekens et al. J Clin Oncol 24:1665-1671, 2006) after adjuvant HT (Ma et al. Cancer Cell 5:607-616, 2004; Paik et al. N Engl J Med 351:2817-2826, 2004; Oh et al. J Clin Oncol 24:1656-1664, 2006) and after neo-adjuvant CT (Sorlie et al. Proc Natl Acad Sci USA 98:10869-10874, 2001; Sorlie et al. Proc Natl Acad Sci USA 100:8418-8423, 2003). A few studies directly analyzed the response to primary CT (Ayers et al. J Clin Oncol 22:2284-2293, 2004; Bertucci et al. Cancer Res 64:8558-8565, 2004; Chang et al. Lancet 362:362-369, 2003; Hannemann et al. J Clin Oncol. 23:3331-3342, 2005). Only few data with small (Bertucci al. Lancet 360:173-174; discussion 174, 2002; Bertucci et al. Hum Mol Genet 9:2981-2991, 2000) or heterogeneous series (Pawitan et al. Breast Cancer Res 7:R953-964, 2005) are available regarding outcome after adjuvant CT. In all these studies, the prognostic and/or predictive multigenic signatures appeared more performing than individual molecular and pathoclinical parameters.

There is a need of adapting adjuvant CT in patients that are candidate to CT. The ongoing introduction of new drugs in adjuvant setting—in general associated to a low and heterogeneous benefit and a morbid and financial cost—necessitates refining the assessment of the metastatic risk after a given CT regimen and the decision regarding what CT regimen to use.

After exhausting testing we have identified gene marker sets that predict clinical outcome after CT, and methods of use thereof. This represents a step towards molecular tailoring by guiding patients towards the most beneficial CT regimen. This would allow moving away from the “one shoe fits all” strategy used in oncology for many years and from the ongoing therapeutic escalation.

### SUMMARY OF THE INVENTION

The invention relates to a method for assessing the clinical outcome of a female mammal suffering from breast cancer, comprising the step of:

a) generating a metagene adjusted value underER by comparing the expression level, in a biological sample from said female mammal and in a control, of at least 10 nucleic acid sequences selected in the group comprising or consisting of: SEQ ID No:374 (nm_{—}000212), SEQ ID No:1027 (nm_{—}007365), SEQ ID No:598 (nm_{—}000636), SEQ ID No:717 (nm_{—}024598), SEQ ID No:573 (nm_{—}001527), SEQ ID No:83 (nm_{—}015065), SEQ ID No:12 (nm_{—}002964), SEQ ID No:405 (nm_{—}000852), SEQ ID No:856 (nm_{—}005564), SEQ ID No:384 (nm_{—}002466), SEQ ID No:167 (nm_{—}002627), SEQ ID No:51 (nm_{—}198433), SEQ ID No:999 (nm_{—}145290), SEQ ID No:979 (nm_{—}004414), SEQ ID No:2 (nm_{—}005245), SEQ ID No:98 (nm_{—}016267), SEQ ID No:751 (nm_{—}002423), SEQ ID No:696 (nm_{—}001428), SEQ ID No:1050 (BC034638), SEQ ID No:488 (nm_{—}002979), SEQ ID No:262 (nm_{—}005194), SEQ ID No:1020 (nm_{—}000359), SEQ ID No:1106 (BC015969), SEQ ID No:952 (nm_{—}003878), SEQ ID No:675 (nm_{—}001512), SEQ ID No:289 (nm_{—}020179), SEQ ID No:553 (nm_{—}004701), SEQ ID No:579 (nm_{—}001814), SEQ ID No:760 (nm_{—}005746), SEQ ID No:805 (nm_{—}014624), SEQ ID No:361 (nm_{—}002906), SEQ ID No:448 (nm_{—}198569), SEQ ID No:170 (nm_{—}002428), SEQ ID No:878 (nm_{—}002774), SEQ ID No:1117, SEQ ID No:612 (nm_{—}032515), SEQ ID No:540 (nm_{—}003159), SEQ ID No:823 (nm_{—}000100), SEQ ID No:131 (nm_{—}145280), SEQ ID No:705 (nm_{—}005596), SEQ ID No:31 (nm_{—}005558), and SEQ ID No:199 (nm_{—}024323), fragments, derivatives or complementary sequences thereof.

Preferably, at least 20 nucleic acid sequences selected in said group, and more preferably at least 25 nucleic acid sequences selected in said group.

In one embodiment, said metagene adjusted value underER is generated by comparing the expression level, in a biological sample from said female mammal and in a control, of the 20 nucleic acid sequences selected in the group consisting of: SEQ ID No:374 (nm_{—}000212); SEQ ID No:1027 (nm_{—}007365); SEQ ID No:598 (nm_{—}000636); SEQ ID No:573 (nm_{—}001527); SEQ ID No:83 (nm_{—}015065); SEQ ID No:12 (nm_{—}002964); SEQ ID No:405 (nm_{—}000852); SEQ ID No:856 (nm_{—}005564); SEQ ID No:167 (nm_{—}002627); SEQ ID No:51 (nm_{—}198433); SEQ ID No:98 (nm_{—}016267); SEQ ID No:751 (nm_{—}002423); SEQ ID No:696 (nm_{—}001428); SEQ ID No:262 (nm_{—}005194); SEQ ID No:1020 (nm_{—}000359); SEQ ID No:579 (nm_{—}001814); SEQ ID No:760 (nm_{—}005746); SEQ ID No:805 (nm_{—}014624); SEQ ID No:878 (nm_{—}002774); and SEQ ID No:612 (nm_{—}032515), fragments, derivatives or complementary sequences thereof.

In another embodiment, said metagene adjusted value underER is generated by comparing the expression level, in a biological sample from said female mammal and in a control, of the 27 nucleic acid sequences selected in the group consisting of: SEQ ID No:374 (nm_{—}000212); SEQ ID No:1027 (nm_{—}007365); SEQ ID No:598 (nm_{—}000636); SEQ ID No:573 (nm_{—}001527); SEQ ID No:83 (nm_{—}015065); SEQ ID No:12 (nm_{—}002964); SEQ ID No:405 (nm_{—}000852); SEQ ID No:856 (nm_{—}005564); SEQ ID No:167 (nm_{—}002627); SEQ ID No:51 (nm_{—}198433); SEQ ID No:98 (nm_{—}016267); SEQ ID No:751 (nm_{—}002423); SEQ ID No:696 (nm_{—}001428); SEQ ID No:262 (nm_{—}005194); SEQ ID No:1020 (nm_{—}000359); SEQ ID No:579 (nm_{—}001814); SEQ ID No:760 (nm_{—}005746); SEQ ID No:805 (nm_{—}014624); SEQ ID No:878 (nm_{—}002774); SEQ ID No:612 (nm_{—}032515); SEQ ID No:384 (nm_{—}002466); SEQ ID No:2 (nm_{—}005245); SEQ ID No:1050 (BC034638); SEQ ID No:952 (nm_{—}003878); SEQ ID No:361 (nm_{—}002906); SEQ ID No:31 (nm_{—}005558); and SEQ ID No:199 (nm_{—}024323), fragments, derivatives or complementary sequences thereof.

b) generating a metagene adjusted value underPR by comparing the expression level, in a biological sample from said female mammal and in a control, of at least 6 nucleic acid sequences selected in the group comprising or consisting of: SEQ ID No:598 (nm_{—}000636), SEQ ID No:1122, SEQ ID No:364 (nm_{—}002253), SEQ ID No:387 (nm_{—}006563), SEQ ID No:34 (nm_{—}001229), SEQ ID No:657 (nm_{—}000633), SEQ ID No:384 (nm_{—}002466), SEQ ID No:451 (nm_{—}001110), SEQ ID No:999 (nm_{—}145290), SEQ ID No:1056 (AK126297), SEQ ID No:15 (nm_{—}003243), SEQ ID No:1090 (AK125808), SEQ ID No:1120, SEQ ID No:12 (nm_{—}002964), SEQ ID No:743 (nm_{—}006875), SEQ ID No:414 (nm_{—}000546), SEQ ID No:374 (nm_{—}000212), SEQ ID No:711 (nm_{—}002291), SEQ ID No:663 (nm_{—}006928), SEQ ID No:1102 (AK124587), SEQ ID No:237 (nm_{—}002644), SEQ ID No:60 (nm_{—}022640), SEQ ID No:361 (nm_{—}002906), SEQ ID No:119 (nm_{—}004730) (or SEQ ID No:1109 (NM_{—}002019)), SEQ ID No:167 (nm_{—}002627), SEQ ID No:339 (nm_{—}144970), SEQ ID No:333 (nm_{—}145037), SEQ ID No:83 (nm_{—}015065), SEQ ID No:330 (nm_{—}018291), SEQ ID No:1024 (nm_{—}030666), SEQ ID No:229 (nm_{—}004586), SEQ ID No:925 (nm_{—}005257), SEQ ID No:788 (nm_{—}001005369), SEQ ID No:1104 (AK128524), SEQ ID No:1103 (BX108410), SEQ ID No:66 (nm_{—}000416), SEQ ID No:1030 (nm_{—}024007), SEQ ID No:1119, SEQ ID No:1068 (AK024670), SEQ ID No:241 (nm_{—}000801), SEQ ID No:398 (nm_{—}003084), SEQ ID No:74 (nm_{—}000878), SEQ ID No:1087 (AK074131), SEQ ID No:955 (nm_{—}001986), SEQ ID No:71 (nm_{—}004633), SEQ ID No:1105 (BC072392), SEQ ID No:856 (nm_{—}005564), SEQ ID No:231 (nm_{—}006678), SEQ ID No:593 (nm_{—}001511), SEQ ID No:384 (nm_{—}002466), SEQ ID No:519 (nm_{—}020125), SEQ ID No:579 (nm_{—}001814), SEQ ID No:1039 (nm_{—}006209), SEQ ID No:31 (nm_{—}005558), SEQ ID No:327 (nm_{—}173825), SEQ ID No:573 (nm_{—}001527), SEQ ID No:98 (nm_{—}016267), SEQ ID No:1059 (AK091113), SEQ ID No:886 (nm_{—}000075), SEQ ID No:1032 (nm_{—}005688), SEQ ID No:1091 (XM_{—}378178), SEQ ID No:233 (nm_{—}178155), SEQ ID No:938 (nm_{—}003012), SEQ ID No:264 (nm_{—}152862), SEQ ID No:546 (nm_{—}005874), SEQ ID No:1099 (BC066343) SEQ ID No:1037 (nm_{—}023068), SEQ ID No:550 (nm_{—}004848), SEQ ID No:1027 (nm_{—}007365), SEQ ID No:1005 (nm_{—}014938), SEQ ID No:820 (nm_{—}000593), and SEQ ID No:370 (nm_{—}000106), fragments, derivatives or complementary sequences thereof.

Preferably, at least 10 nucleic acid sequences selected in said group, as an example at least 20 nucleic acid sequences or at least 30 nucleic acid sequences, and more preferably at least 36 nucleic acid sequences selected in said group.

In one embodiment, said metagene adjusted value underPR is generated by comparing the expression level, in a biological sample from said female mammal and in a control, of the 6 nucleic acid sequences selected in the group consisting of: SEQ ID No:364 (nm_{—}002253); SEQ ID No:34 (nm_{—}001229); SEQ ID No:657 (nm_{—}000633); SEQ ID No:339 (nm_{—}144970); SEQ ID No:229 (nm_{—}004586); SEQ ID No:1119, fragments, derivatives or complementary sequences thereof.

In another embodiment, said metagene adjusted value underPR is generated by comparing the expression level, in a biological sample from said female mammal and in a control, of the 36 nucleic acid sequences selected in the group consisting of: SEQ ID No:364 (nm_{—}002253); SEQ ID No:34 (nm_{—}001229); SEQ ID No:657 (nm_{—}000633); SEQ ID No:339 (nm_{—}144970); SEQ ID No:229 (nm_{—}004586); SEQ ID No:1119; SEQ ID No:387 (nm_{—}006563); SEQ ID No:1056 (AK126297); SEQ ID No:15 (nm_{—}003243); SEQ ID No:1120; SEQ ID No:414 (nm_{—}000546); SEQ ID No:374 (nm_{—}000212); SEQ ID No:711 (nm_{—}002291); SEQ ID No:663 (nm_{—}006928); SEQ ID No:237 (nm_{—}002644); SEQ ID No:60 (nm_{—}022640); SEQ ID No:119 (nm_{—}004730); SEQ ID No:330 (nm_{—}018291); SEQ ID No:1024 (nm_{—}030666); SEQ ID No:925 (nm_{—}005257); SEQ ID No:1104 (AK128524); SEQ ID No:1103 (BX108410); SEQ ID No:66 (nm_{—}000416); SEQ ID No:1068 (AK024670); SEQ ID No:374 (nm_{—}000212); SEQ ID No:74 (nm_{—}000878); SEQ ID No:231 (nm_{—}006678); SEQ ID No:593 (nm_{—}001511); SEQ ID No:384 (nm_{—}002466); SEQ ID No:1039 (nm_{—}006209); SEQ ID No:327 (nm_{—}173825); SEQ ID No:886 (nm_{—}000075); SEQ ID No:1032 (nm_{—}005688); SEQ ID No:264 (nm_{—}152862); SEQ ID No:1037 (nm_{—}023068); and SEQ ID No:1005 (nm_{—}014938), fragments, derivatives or complementary sequences thereof.

c) generating a metagene adjusted value underEGFR by comparing the level, in a biological sample from said female mammal and in a control, of at least 10 nucleic acid sequences selected in the group comprising or consisting of: SEQ ID No:1071 (NM_{—}001033047), SEQ ID No:254 (nm_{—}005581), SEQ ID No:6 (nm_{—}003225), SEQ ID No:883 (nm_{—}000125), SEQ ID No:543 (nm_{—}005080), SEQ ID No:681 (nm_{—}020974), SEQ ID No:63 (nm_{—}001002295), SEQ ID No:212 (nm_{—}024852), SEQ ID No:635 (nm_{—}001002029), SEQ ID No:535 (nm_{—}003226), SEQ ID No:1125, SEQ ID No:109 (nm_{—}000662), SEQ ID No:342 (nm_{—}001846), SEQ ID No:927 (nm_{—}004703), SEQ ID No:1124, SEQ ID No:124 (nm_{—}014899), SEQ ID No:280 (nm_{—}020764) (or SEQ ID No:1110 (nm_{—}024522)), SEQ ID No:297 (nm_{—}016463), SEQ ID No:791 (nm_{—}016835), SEQ ID No:210 (nm_{—}178840), SEQ ID No:827 (nm_{—}152499), SEQ ID No:1064 (nm_{—}000767), SEQ ID No:147 (nm_{—}014675), SEQ ID No:323 (nm_{—}001014443), SEQ ID No:106 (nm_{—}004619), SEQ ID No:181 (nm_{—}000848), SEQ ID No:376 (nm_{—}057158), SEQ ID No:116 (nm_{—}014034), SEQ ID No:252 (nm_{—}000758), SEQ ID No:797 (nm_{—}022131), SEQ ID No:911 (nm_{—}000168), SEQ ID No:720 (nm_{—}004726), SEQ ID No:889 (nm_{—}000561), SEQ ID No:250 (nm_{—}000930), SEQ ID No:179 (nm_{—}004747), SEQ ID No:786 (nm_{—}033388), SEQ ID No:177 (nm_{—}015996), SEQ ID No:1047 (BC012900), SEQ ID No:301 (nm_{—}004326), SEQ ID No:207 (nm_{—}003940), SEQ ID No:936 (nm_{—}003462), SEQ ID No:916 (nm_{—}001453) (or SEQ ID No:1116 (nm_{—}004040)), SEQ ID No:1052 (BX096026), SEQ ID No:159 (nm_{—}000224), SEQ ID No:1096 (AK127274), SEQ ID No:28 (nm_{—}021800), SEQ ID No:1054 (AK123264), SEQ ID No:25 (nm_{—}012391) (or SEQ ID No:1108 (nm_{—}053279)), SEQ ID No:825 (nm_{—}024704), SEQ ID No:145 (nm_{—}017786), SEQ ID No:491 (nm_{—}004374), SEQ ID No:485 (nm_{—}003834), SEQ ID No:1072 (AY007114), SEQ ID No:274 (nm_{—}032108), SEQ ID No:258 (nm_{—}080545), SEQ ID No:292 (nm_{—}014371), SEQ ID No:803 (nm_{—}183047), SEQ ID No:349 (nm_{—}031946), SEQ ID No:1123, SEQ ID No:763 (nm_{—}014585), SEQ ID No:438 (nm_{—}001759), SEQ ID No:94 (nm_{—}014315), SEQ ID No:845 (nm_{—}001089), SEQ ID No:1084 (BX648964), SEQ ID No:734 (nm_{—}025137), SEQ ID No:943 (nm_{—}002141), SEQ ID No:1085 (nm_{—}000720), and SEQ ID No:276 (nm_{—}012202), fragments, derivatives or complementary sequences thereof.

Preferably, at least 20 nucleic acid sequences selected in said group, as an example at least 24 nucleic acid sequences or at least 30 nucleic acid sequences, and more preferably at least 37 nucleic acid sequences selected in said group.

In one embodiment, said metagene adjusted value underEGFR is generated by comparing the expression level, in a biological sample from said female mammal and in a control, of the 24 nucleic acid sequences selected in the group consisting of: SEQ ID No:1071 (nm_{—}001033047); SEQ ID No:254 (nm_{—}005581); SEQ ID No:6 (nm_{—}003225); SEQ ID No:883 (nm_{—}000125); SEQ ID No:543 (nm_{—}005080); SEQ ID No:681 (nm_{—}020974); SEQ ID No:63 (nm_{—}001002295); SEQ ID No:212 (nm_{—}024852); SEQ ID No:635 (nm_{—}001002029); SEQ ID No:535 (nm_{—}003226); SEQ ID No:1125); SEQ ID No:1124; SEQ ID No:297 (nm_{—}016463); SEQ ID No:791 (nm_{—}016835); SEQ ID No:827 (nm_{—}152499); SEQ ID No:207 (nm_{—}003940); SEQ ID No:916 (nm_{—}001453) (or SEQ ID No:1116 (nm_{—}004040)); SEQ ID No:1052 (BX096026); SEQ ID No:159 (nm_{—}000224); SEQ ID No:25 (nm_{—}012391) (or SEQ ID No:1108 (nm_{—}053279)); SEQ ID No:845 (nm_{—}001089); and SEQ ID No:1085 (nm_{—}000720), fragments, derivatives or complementary sequences thereof.

In another embodiment, said metagene adjusted value underEGFR is generated by comparing the expression level, in a biological sample from said female mammal and in a control, of the 37 nucleic acid sequences selected in the group consisting of: SEQ ID No:1071 (nm_{—}001033047); SEQ ID No:254 (nm_{—}005581); SEQ ID No:6 (nm_{—}003225); SEQ ID No:883 (nm_{—}000125); SEQ ID No:543 (nm_{—}005080); SEQ ID No:681 (nm_{—}020974); SEQ ID No:63 (nm_{—}001002295); SEQ ID No:212 (nm_{—}024852); SEQ ID No:635 (nm_{—}001002029); SEQ ID No:535 (nm_{—}003226); SEQ ID No:1125; SEQ ID No:1124; SEQ ID No:297 (nm_{—}016463); SEQ ID No:791 (nm_{—}016835); SEQ ID No:827 (nm_{—}152499); SEQ ID No:207 (nm_{—}003940); SEQ ID No:916 (nm_{—}001453) (or SEQ ID No:1116 (nm_{—}004040)); SEQ ID No:1052 (BX096026); SEQ ID No:159 (nm_{—}000224); SEQ ID No:25 (nm_{—}012391) (or SEQ ID No:1108 (NM_{—}053279)); SEQ ID No:845 (nm_{—}001089); SEQ ID No:1085 (NM_{—}000720); SEQ ID No:109 (nm_{—}000662); SEQ ID No:342 (nm_{—}001846); SEQ ID No:927 (nm_{—}004703); SEQ ID No:280 (nm_{—}020764) (or SEQ ID No:1110 (NM_{—}024522)); SEQ ID No:210 (nm_{—}178840); SEQ ID No:181 (nm_{—}000848); SEQ ID No:116 (nm_{—}014034); SEQ ID No:250 (nm_{—}000930); SEQ ID No:177 (nm_{—}015996); SEQ ID No:825 (nm_{—}024704); SEQ ID No:145 (nm_{—}017786); and SEQ ID No:276 (nm_{—}012202), fragments, derivatives or complementary sequences thereof.

d) generating a score (S_{C}) from said metagene adjusted values using a mathematical method establishing a relation between the combined metagene values and the clinical outcome of said female mammal.

In one embodiment, the mathematical method used in step d) comprises a Cox regression analysis (Wright et al., Proc. Natl. Acad. Sci. USA, vol. 100 (17), p. 9991-9996, 2003) or a CART analysis (Breiman et al Classification and Regression Trees, Chapman & Hall 1984).

In a particular embodiment, the mathematical method is a Cox regression analysis and the score (S_{C}) is generated according to the following formula: S_{C}=a×underER+b×underPR+c×under EGFR, wherein “a” is comprised in the interval [−6.26; +0.49], “b” is comprised in the interval [−2.65; +0.29] and “c” is comprised in the interval [−6.69; +1.65].

For example the formula is: S_{C}=−2.90279×underER−1.47423×underPR−4.17198×under EGFR.

The invention further relates to a method for assessing the clinical outcome of a female mammal suffering from breast cancer, comprising the step of:

a) generating a metagene adjusted value underEGFR by comparing the expression level, in a biological sample from said female mammal and in a control, of at least one nucleic acid sequence selected in the group consisting of: SEQ ID No:1071 (NM_{—}001033047), SEQ ID No:254 (nm_{—}005581), SEQ ID No:6 (nm_{—}003225), SEQ ID No:883 (nm_{—}000125), SEQ ID No:543 (nm_{—}005080), SEQ ID No:681 (nm_{—}020974), SEQ ID No:63 (nm_{—}001002295), SEQ ID No:212 (nm_{—}024852), SEQ ID No:635 (nm_{—}001002029), SEQ ID No:535 (nm_{—}003226), SEQ ID No:1125, SEQ ID No:109 (nm_{—}000662), SEQ ID No:342 (nm_{—}001846), SEQ ID No:927 (nm_{—}004703), SEQ ID No:1124, SEQ ID No:124 (nm_{—}014899), SEQ ID No:280 (nm_{—}020764) (or SEQ ID No:1110 (nm_{—}024522)), SEQ ID No:297 (nm_{—}016463), SEQ ID No:791 (nm_{—}016835), SEQ ID No:210 (nm_{—}178840), SEQ ID No:827 (nm_{—}152499), SEQ ID No:1064 (NM_{—}000767), SEQ ID No:147 (nm_{—}014675), SEQ ID No:323 (nm_{—}001014443), SEQ ID No:106 (nm_{—}004619), SEQ ID No:181 (nm_{—}000848), SEQ ID No:376 (nm_{—}057158), SEQ ID No:116 (nm_{—}014034), SEQ ID No:252 (nm_{—}000758), SEQ ID No:797 (nm_{—}022131), SEQ ID No:911 (nm_{—}000168), SEQ ID No:720 (nm_{—}004726), SEQ ID No:889 (nm_{—}000561), SEQ ID No:250 (nm_{—}000930), SEQ ID No:179 (nm_{—}004747), SEQ ID No:786 (nm_{—}033388), SEQ ID No:177 (nm_{—}015996), SEQ ID No:1047 (BC012900), SEQ ID No:301 (nm_{—}004326), SEQ ID No:207 (nm_{—}003940), SEQ ID No:936 (nm_{—}003462), SEQ ID No:916 (nm_{—}001453) (or SEQ ID No:1116 (NM_{—}004040)), SEQ ID No:1052 (BX096026), SEQ ID No:159 (nm_{—}000224), SEQ ID No:1096 (AK127274), SEQ ID No:28 (nm_{—}021800), SEQ ID No:1054 (AK123264), SEQ ID No:25 (nm_{—}012391) (or SEQ ID No:1108 (nm_{—}053279)), SEQ ID No:825 (nm_{—}024704), SEQ ID No:145 (nm_{—}017786), SEQ ID No:491 (nm_{—}004374), SEQ ID No:485 (nm_{—}003834), SEQ ID No:1072 (AY007114), SEQ ID No:274 (nm_{—}032108), SEQ ID No:258 (nm_{—}080545), SEQ ID No:292 (nm_{—}014371), SEQ ID No:803 (nm_{—}183047), SEQ ID No:349 (nm_{—}031946), SEQ ID No:1123, SEQ ID No:763 (nm_{—}014585), SEQ ID No:438 (nm_{—}001759), SEQ ID No:94 (nm_{—}014315), SEQ ID No:845 (nm_{—}001089), SEQ ID No:1084 (BX648964), SEQ ID No:734 (nm_{—}025137), SEQ ID No:943 (nm_{—}002141), SEQ ID No:1085 (nm_{—}000720), and SEQ ID No:276 (nm_{—}012202), fragments, derivatives or complementary sequences thereof.

Preferably, said nucleic acid sequence is SEQ ID No:681 (nm_{—}020974), fragments, derivatives or complementary sequences thereof.

Preferably, at least 10 nucleic acid sequences selected in said group, as an example at least 20 nucleic acid sequences or at least 24 nucleic acid sequences, and more preferably at least 37 nucleic acid sequences selected in said group.

In one embodiment, said metagene adjusted value underEGFR is generated by comparing the expression level, in a biological sample from said female mammal and in a control, of the 24 nucleic acid sequences selected in the group consisting of: SEQ ID No:1071 (nm_{—}001033047); SEQ ID No:254 (nm_{—}005581); SEQ ID No:6 (nm_{—}003225); SEQ ID No:883 (nm_{—}000125); SEQ ID No:543 (nm_{—}005080); SEQ ID No:681 (nm_{—}020974); SEQ ID No:63 (nm_{—}001002295); SEQ ID No:212 (nm_{—}024852); SEQ ID No:635 (nm_{—}001002029); SEQ ID No:535 (nm_{—}003226); SEQ ID No:1125); SEQ ID No:1124; SEQ ID No:297 (nm_{—}016463); SEQ ID No:791 (nm_{—}016835); SEQ ID No:827 (nm_{—}152499); SEQ ID No:207 (nm_{—}003940); SEQ ID No:916 (nm_{—}001453) (or SEQ ID No:1116 (nm_{—}004040)); SEQ ID No:1052 (BX096026); SEQ ID No:159 (nm_{—}000224); SEQ ID No:25 (nm_{—}012391) (or SEQ ID No:1108 (NM_{—}053279)); SEQ ID No:845 (nm_{—}001089); and SEQ ID No:1085 (NM_{—}000720), fragments, derivatives or complementary sequences thereof.

In another embodiment, said metagene adjusted value underEGFR is generated by comparing the expression level, in a biological sample from said female mammal and in a control, of the 37 nucleic acid sequences selected in the group consisting of: SEQ ID No:1071 (nm_{—}001033047); SEQ ID No:254 (nm_{—}005581); SEQ ID No:6 (nm_{—}003225); SEQ ID No:883 (nm_{—}000125); SEQ ID No:543 (nm_{—}005080); SEQ ID No:681 (nm_{—}020974); SEQ ID No:63 (nm_{—}001002295); SEQ ID No:212 (nm_{—}024852); SEQ ID No:635 (nm_{—}001002029); SEQ ID No:535 (nm_{—}003226); SEQ ID No:1125; SEQ ID No:1124; SEQ ID No:297 (nm_{—}016463); SEQ ID No:791 (nm_{—}016835); SEQ ID No:827 (nm_{—}152499); SEQ ID No:207 (nm_{—}003940); SEQ ID No:916 (nm_{—}001453) (or SEQ ID No:1116 (nm_{—}004040)); SEQ ID No:1052 (BX096026); SEQ ID No:159 (nm_{—}000224); SEQ ID No:25 (nm_{—}012391) (or SEQ ID No:1108 (NM_{—}053279)); SEQ ID No:845 (nm_{—}001089); SEQ ID No:1085 (NM_{—}000720); SEQ ID No:109 (nm_{—}000662); SEQ ID No:342 (nm_{—}001846); SEQ ID No:927 (nm_{—}004703); SEQ ID No:280 (nm_{—}020764) (or SEQ ID No:1110 (NM_{—}024522)); SEQ ID No:210 (nm_{—}178840); SEQ ID No:181 (nm_{—}000848); SEQ ID No:116 (nm_{—}014034); SEQ ID No:250 (nm_{—}000930); SEQ ID No:177 (nm_{—}015996); SEQ ID No:825 (nm_{—}024704); SEQ ID No:145 (nm_{—}017786); and SEQ ID No:276 (nm_{—}012202), fragments, derivatives or complementary sequences thereof.

b) generating a metagene adjusted value overEGFR by comparing the expression level, in a biological sample from said female mammal and in a control, of at least one nucleic acid sequences selected in the group consisting of SEQ ID No:405 (nm_{—}000852), SEQ ID No:374 (nm_{—}000212), SEQ ID No:1122, SEQ ID No:598 (nm_{—}000636), SEQ ID No:262 (nm_{—}005194), SEQ ID No:1099 (BC066343), SEQ ID No:696 (nm_{—}001428), SEQ ID No:1059 (AK091113), SEQ ID No:751 (nm_{—}002423), SEQ ID No:1121, SEQ ID No:286 (nm_{—}002417), SEQ ID No:244 (nm_{—}199002), SEQ ID No:18 (nm_{—}001880), SEQ ID No:121 (nm_{—}014553), SEQ ID No:1107 (BC073775), SEQ ID No:103 (nm_{—}003619), SEQ ID No:1118, SEQ ID No:42 (nm_{—}000757), and SEQ ID No:1067 (AK123784), fragments, derivatives or complementary sequences thereof.

Preferably, said nucleic acid sequence is SEQ ID No: 1107 (BC073775) or SEQ ID No: 1099 (BC066343), fragments, derivatives or complementary sequences thereof.

More preferably, at least 5 nucleic acid sequences selected in said group, as an example at least 10 nucleic acid sequences, and more preferably at least 12 nucleic acid sequences selected in said group.

In one embodiment, said metagene adjusted value overEGFR is generated by comparing the expression level, in a biological sample from said female mammal and in a control, of the 5 nucleic acid sequences selected in the group consisting of: SEQ ID No:1122; SEQ ID No:598 (nm_{—}000636); SEQ ID No:696 (nm_{—}001428); SEQ ID No:1059 (AK091113); and SEQ ID No:121 (nm_{—}014553), fragments, derivatives or complementary sequences thereof.

In another embodiment, said metagene adjusted value overEGFR is generated by comparing the expression level, in a biological sample from said female mammal and in a control, of the 12 nucleic acid sequences selected in the group consisting of: SEQ ID No:1122; SEQ ID No:598 (nm_{—}000636); SEQ ID No:696 (nm_{—}001428); SEQ ID No:1059 (AK091113); SEQ ID No:121 (nm_{—}014553); SEQ ID No:262 (nm_{—}005194); SEQ ID No:1099 (BC066343); SEQ ID No:751 (nm_{—}002423); SEQ ID No:1121; SEQ ID No:286 (nm_{—}002417); SEQ ID No:103 (nm_{—}003619); and SEQ ID No:1118, fragments, derivatives or complementary sequences thereof.

c) generating a score (S_{C}) from said metagene adjusted values using a mathematical method establishing a relation between the combined metagene values and the clinical outcome of said female mammal.

In one embodiment, the mathematical method used in step c) comprises a Cox regression analysis or a CART analysis.

In another embodiment, the mathematical method is a Cox regression and the score (S_{C}) to the following formula: S_{C}=a×overEGFR+b×underEGFR, wherein “a” is comprised in the interval [−1.85; +0.81] and “b” is comprised in the interval [−3.86; +0.70]

For example the formula is: S_{C}=−1.33×over EGFR×2.28×under EGFR.

The invention further relates to a method of assessing the clinical outcome of a female mammal suffering from breast cancer, comprising the steps of:

a) generating a metagene adjusted value underER by comparing the expression level, in a biological sample from said female mammal and in a control, of at least two genes, e.g. by using nucleic acid sequences selected in the group of Affymetrix® Probe Sets, of table IX or XII, preferably table XII (described below),

b) generating said metagene adjusted value underPR by comparing the expression level, in a biological sample from said female mammal and in a control, of at least two genes, e.g. by using nucleic acid sequences selected in the group of Affymetrix® Probe Sets, of table X or XIII, preferably table XIII (described below),

c) generating said metagene adjusted value underEGFR by comparing the expression level, in a biological sample from said female mammal and in a control, of at least two genes, e.g. by using the nucleic acid sequences selected in the group of Affymetrix® Probe Sets, of table XI or XIV, preferably table XIV (described below),

d) generating a score (S_{C}) from said metagene adjusted values using a mathematical method establishing a relation between the combined metagene values and the clinical outcome of said female mammal.

In one embodiment, the mathematical method used in step d) comprises a Cox regression or CART analysis.

In another embodiment, the mathematical method used in step d) is a Cox regression and the score (S_{C}) is generated according to the following formula: S_{C}=a×underER+b×underPR+c×under EGFR, wherein “a” is comprised in the interval [−6.26; +0.49], “b” is comprised in the interval [−2.65; +0.29] and “c” is comprised in the interval [−6.69; +1.65].

For example, the formula is: S_{C}=−2.90279×underER−1.47423×underPR−4.17198×under EGFR.

Preferably, the comparing of expression level at each step a), b) and c) is performed with at least 5, preferably 10, preferably all of said genes or nucleic acid sequences of each respective group.

In various embodiments, said methods may comprise the first step of quantifying in a biological sample from said female mammal the expression level of said nucleic acids sequences.

In other various embodiments, these methods can comprise the step e) of comparing said score (S_{C}) from the biological sample with a baseline or a score (S_{C}) from a control sample.

In other various embodiments, said biological sample is a breast tumor sample. By “sample” is meant a cell or a tissue.

In other various embodiments, said methods further comprise a step of taking at least one biological sample from said female mammal.

In another embodiment, said methods comprise a step of administrating a pharmaceutical treatment, preferably a chemotherapy treatment to a female mammal, for optimizing the clinical outcome of said female mammal in response to said treatment. The pharmaceutical treatment may comprise the use of one or more taxane compounds, e.g., docetaxel or paclitaxel. This treatment may be administered if the female mammal has not responded to a previous anti-cancer treatment, e.g., a treatment comprising the use of one or more anthracyclin compound, e.g., epirubicin, doxorubicin, pirarubicin, idarubicin, zorubicin or aclarubicin, preferably epirubicin.

In a further aspect, the methods according to the invention may be used for identifying a female mammal that has not responded to a previous anti-cancer treatment, e.g., a treatment comprising the use of one or more anthracyclin compound, e.g., epirubicin, doxorubicin, pirarubicin, idarubicin, zorubicin or aclarubicin, preferably epirubicin.

In other various embodiments, a comparison of or analysis of data may involve a statistical computer mediated analysis. Also, said methods may optionally further involve generating a printed report.

The invention further relates to a computer program comprising instructions for performing said methods.

Finally, the invention relates to a recording medium for recording said computer program.

### DETAILED DESCRIPTION

Unless otherwise noted, technical terms are used according to conventional usage.

In order to facilitate review of the various embodiment of the invention, the following explanation of specific terms is provided:

Mammals corresponds to animals such as humans, mice, rats, guinea pigs, monkeys, cats, dogs, pigs, horses, or cows, preferably to humans, and most preferably to women;

Biological sample: any biological material, such as a cell, a tissue sample, or a biopsy from breast cancer.

A “Metagene” as used herein corresponds to a group of genes for which expression variation (but not necessarily expression level) across tumors is correlated. A metagene can be simply calculated by one of skill in the art according to the method as described in the examples.

A “Control” as used herein corresponds to one or more biological samples from a cell, a tissue sample or a biopsy from breast. Said control may be obtained from the same female mammal than the one to be tested or from another female mammal, preferably from the same specie, or from a population of females mammal, preferably from the same specie, that may be the same or different from the test female mammal or subject. Said control may correspond to a biological sample from a cell, a cell line, a tissue sample or a biopsy from breast cancer. Preferably, the expression of EGFR, RE, PR and/or KI-67 has been established for this biological sample, by IHC (ImmunoHistoChemistry) FISH (Fluorescence In Situ Hybridization) or Quantitative PCR, for example.

In silico research: Literally referring to “in computer” systems, in silico research involves methods to test biological models, drugs, and other interventions using computer models rather than laboratory (in vitro) and animal (in vivo) experiments. In silico methods can involve analyzing an existing database, for instance a database that includes one or more records that include quantitative analysis of nucleic acid sequence expression. Analysis of such databases may include mining, parsing, selecting, identifying, sorting, or filtering of the data in the database. Data in the database can also be subjected to a clustering algorithm, discrimination algorithm, difference test, correlation, regression algorithm or other statistical modeling algorithm.

Using in silico research, drug treatment can be selected, tested and validated, and experimental strategies can be assessed. In silico systems complement laboratory-based research, yet increase productivity and efficiency by minimizing the need for in vitro and in vivo laboratory experiments.

In certain embodiments provided herein, in silico systems are used. In particular, this disclosure provides in silico methods for assessing a condition related to the clinical outcome of a female mammal suffering from breast cancer. Such methods involve assessing data in a database. The data in the database usually includes a quantity of nucleic acids from a biological sample from one or more individuals.

Quantitative data as discussed herein include molar quantitative data or relative data (variation of expression compared to control) for individual nucleic acid sequences, or subsets of nucleic acid sequences. Quantitative aspects of nucleic acids samples may be provided and/or improved by including one or more quantitative internal standards during the analysis, for instance one control nucleic acid sequence. Internal standards described herein enable true quantification of each nucleic acid sequence expression.

Truly quantitative data can be integrated from multiple sources (whether it is work from different labs, samples from different subjects, or merely samples processed on different days) into a single seamless database, regardless of the number of nucleic acid sequences measured in each discrete, individual analysis.

In any of the provided methods, a comparison of or an analysis involves a statistical or computer-mediated analysis.

The mathematical model (or method) for establishing a relation between the combined metagene adjusted values is realized on a population of mammal females showing the same ethnic and the same breast cancer characteristics than the female mammal to be tested.

The metagene coefficients (a, b, c) in the formulas used to calculate the scores (S_{C}) may vary according to the used tumor samples database consisting of mammal females showing the same ethnic and the same characteristics. A skilled person may calculate these coefficients by using a so-called Cox regression as described in Wright et al. (Proc. Natl. Acad. Sci. USA, vol. 100 (17), p. 9991-9996, 2003)

Optionally, in some of the provided embodiments, the methods further involve comparing the score (S_{C}) from the female mammal to the score (S_{C}) from another female mammal, preferably from the same specie, or a compiled score (S_{C}) from a population of females mammal, preferably from the same specie, that may be the same or different from the test female mammal or subject.

In specific examples of such methods, the control is a baseline corresponding to a score (S_{C}) established from a population of females mammal.

The baseline is simply determined by one of skill in the art in view of the protocol described in the examples. An optimal baseline is obtained by using score distribution separating tumors into two groups of most significant different outcome.

As an example (described below), the inventors have established that a woman having a score (S_{C}) of more than 0.136 have at least a double propensity of poor clinical outcome than a woman with a score (S_{C}) of less than 0.0393.

Any of the provided method can further involve generating a printed report, for instance a report of some or all the data, of some or all the conclusions drawn from the data, or of a score or comparison between the results of a subject or individual and other individuals or a control or baseline.

There are many ways to collect quantitative or relative data on nucleic acids sequences, and the analytical methodology does not affect the utility of nucleic acids sequences expression in assessing the clinical outcome of a female mammal suffering from breast cancer. Methods for determining quantities of nucleic acids expression in a biological sample are well known from one of skill in the art. As an example of such methods, one can cite northern blot, cDNA array, oligo arrays or quantitative Reverse Transcription-PCR.

Preferably said methodology is cDNA arrays or oligo arrays, which allows the quantitative study of numerous candidate genes mRNA expression levels.

DNA arrays consist of large numbers of DNA molecules spotted in a systematic order on a solid support or substrate such as a nylon membrane, glass slide, glass beads or a silicon chip. Depending on the size of each DNA spot on the array, DNA arrays can be categorized as microarrays (each DNA spot has a diameter less than 250 microns) and macroarrays (spot diameter is grater than 300 microns). When the solid substrate used is small in size, arrays are also referred to as DNA chips. Depending on the spotting technique used, the number of spots on a glass microarray can range from hundreds to thousands.

Typically, a method of monitoring gene expression by DNA array involves the following steps:

a) obtaining a polynucleotide sample from a subject; and

b) reacting the sample polynucleotide obtained in step (a) with a probe immobilized on a solid support wherein said probe consist of polynucleotides having the nucleic acids sequence as previously described, fragments, derivative or complementary sequence thereof.

c) detecting the reaction product of step (b).

In the present invention, the term “polynucleotide” refers to a polymer of RNA or DNA that is single- or double-stranded, optionally containing synthetic, non-natural or altered nucleotide bases. A polynucleotide in the form of a polymer of DNA may be comprised of one or more segments of cDNA, genomic DNA or synthetic DNA.

In the present invention, the term “fragment” refers to a sequence of nucleic acids that allows a specific hybridization under stringent conditions, as an example more than 10 nucleotides, preferably more than 15 nucleotides, and most preferably more than 25 nucleotides, as an example more than 50 nucleotides or more than 100 nucleotides.

In the present invention, the term “derivative” refers to a sequence having more than 80% identity with an identified nucleic acid sequence, preferably more than 90% identity, as an example more than 95% identity, and most particularly more than 99% identity.

In the present invention, the term “immobilized on a support” means bound directly or indirectly thereto including attachment by covalent binding, hydrogen bonding, ionic interaction, hydrophobic interaction or otherwise.

The polynucleotide sample isolated from the subject and obtained at step (a) is RNA, preferably mRNA. Said polynucleotide sample isolated from the patient can also correspond to cDNA obtained by reverse transcription of the mRNA, or a product of ligation after specific hybridization of specific probes to mRNA or cDNA.

Preferably, the polynucleotide sample obtained at step (a) is labeled before its reaction at step (b) with the probe immobilized on a solid support. Such labeling is well known from one of skill in the art and includes, but is not limited to, radioactive, colorimetric, enzymatic, molecular amplification, bioluminescent, electrochemical or fluorescent labeling.

Advantageously, the reaction product of step (c) is quantified by further comparison of said reaction product to a control sample.

Detection preferably involves calculating/quantifying a relative expression (transcription) level for each nucleic acids sequence.

Then, the determination of the relative expression level for each nucleic acid sequences previously described enables to assess the clinical outcome of the subject—i.e. female mammal—suffering from breast cancer by the method of the invention.

The method of assessing the clinical outcome of a female mammal suffering from breast cancer can further involve a step of taking a biological sample, preferably breast cancer tissue or cells from a female mammal. Such methods of sampling are well known of one of skill in the art, and as an example, one can cite surgery.

The provided method may also correspond to an in vitro method, which does not include such a step of sampling.

Also provided are methods to determine if a pharmaceutical treatment, especially chemotherapy treatment, influences the clinical outcome of a female mammal suffering from breast cancer, which methods involve quantifying said nucleic acids sequences expression in a biological sample from a female mammal and determining the score (S_{C}) for said female mammal.

Further embodiments are methods to assess or identify a therapeutic or pharmaceutical agent for its potential effectiveness, efficacy or side effects relating to the clinical outcome, which methods involve quantifying said nucleic acids sequences in a biological sample from a female mammal suffering from breast cancer and determining the score (S_{C}) for said female mammal.

Also provided herein are methods of assessing a change in the propensity of clinical outcome from a female mammal suffering from breast cancer, wherein the methods involve taking at least two biological samples from the female mammal, one of which is taken before and one after an event. In various specific embodiments, the event involves passage of time (e.g., minutes, hours, days, weeks, months, or years), treatment with a therapeutic agent (or putative or potential therapeutic agent), treatment with a pharmaceutical agent (or putative or potential pharmaceutical agent).

One specific provided embodiment is a method of determining whether or to what extent a condition influences the clinical outcome of a female mammal suffering from breast cancer. This method involves subjecting a subject to the condition, taking a biological sample from the subject, analyzing the biological sample to produce a score (S_{C}) for said subject, and comparing said score (S_{C}) for the subject with a control. From this comparison, conclusions are drawn about whether or to what extent the condition influences the clinical outcome of female mammal suffering from breast cancer based on differences or similarities between the test score (S_{C}) and the control. As contemplated for this embodiment, a condition to which the subject is subjected can include but is not limited to application of a pharmaceutical or therapeutic agent or candidate agent.

Subject: a female mammal.

In specific examples of such methods, the nucleic acids sequences expression profile is a pre-condition score (S_{C}) from the subject or a compiled score (S_{C}) assembled from a plurality of individual score (S_{C}). In other examples, the control score (S_{C}) is a control or a baseline established from previously described control score (S_{C}).

Pharmaceutical treatment: any agent treatment, regimen, or dosage, such the administration of a protein, a peptide (e.g., hormone), other organic molecule or inorganic molecule or compound, or combination thereof, that has or should have beneficial effects on clinical outcome when properly administrated to a subject, preferably said agents are used in chemotherapy.

Unless otherwise explained, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

In various embodiments, the provided methods further comprise the step of selecting the pharmaceutical treatment that improves the clinical outcome of a female mammal suffering from breast cancer.

The present invention will be understood more clearly on reading the description of the experimental studies performed in the context of the research carried out by the applicant, which should not be interpreted as being limiting in nature.

### Example 1

### Identification of Significant Metagenes Combination

### 1) Goals:

While it is now possible to assess patients' responses to drugs with respect to their genomic profile, the standard adjuvant chemotherapy (anthracyclines and taxanes) for non metastatic breast cancer may not be systematically appropriate: according to their genomic profile, women may rather benefit from a treatment based on anthracyclines alone without taxane.

The primary objective was to identify a gene set, which discriminate two groups of patients with different clinical outcome based on gene expression. This goal was reached by: defining the gene expression profiles, using 9.000-genes microarrays, of 323 tumours obtained from patients treated with adjuvant anthracycline-based CT without taxanes (identification set), grouping individual genes in metagenes and identifying metagenes closely correlated with the biological status of ER, PR, HER2/Neu, MIB/KI67, EGFR status of the sample as determined by the mean of independent methods such as Immunohistochemistry or FISH. Then we combined these metagenes using a Cox proportional hazard ratio analysis to separate patients according to clinical outcome. This latter step providing a model consisting of a score expressed as a linear combination such as Score=Σβᵢ.xᵢ where βᵢ.is a fixed parameter and xᵢ is the value of the metagene.

The secondary objective was to prospectively validate the Cox model and its metagene component for predicting clinical outcome in an independent cohort of patients (validation set). This goal was reached by defining the gene expression profiles of 164 tumours, using the same technology, obtained from patients treated with adjuvant anthracycline-based CT without taxanes in the context of a multicentric clinical trial.

### 2) Patients:

We profiled a multicentric and retrospective series of 504 early breast cancers (Institut Paoli Calmettes, Centre Léon Bérard, Institut Bergonié and tumours from clinicals trials PACS01 and PEGASE01) treated with adjuvant anthracycline-based and non taxane-based CT. Clinical and pathological criteria for each patient are summarized in the following table and correspond to the identification and the validation sets.

Global population demography:

| Age | median (min-max) | 50 | (11-90) |
|---|---|---|---|
| menopausal | Y | 210 | (41.8%) |
| | N | 292 | (58.2%) |
| Tumour size | pT1 | 105 | (21%) |
| | pT2 | 317 | (63.5%) |
| | pT3 | 77 | (15.4%) |
| N (Node) | N− | 67 | (13.3%) |
| | N+ | 437 | (86.7%) |
| Node category | N1 (N = 0) | 67 | (13.3%) |
| | N2 (N = 1 to 3) | 248 | (49.2%) |
| | N3 (N > 3) | 189 | (37.5%) |
| grade SBR | I | 66 | (13.4%) |
| | II | 221 | (45%) |
| | III | 204 | (41.5%) |
| RE (10%) | RE− | 150 | (31%) |
| (Estrogen Receptor) | RE+ | 334 | (69%) |
| RP (10%) | RP− | 199 | (41.5%) |
| (Progesterone Receptor) | RP+ | 280 | (58.5%) |
| RH (10%) | RH− | 115 | (23.8%) |
| (Hormone Receptor; RE | RH+ | 369 | (76.2%) |
| and/or RP) | | | |
| Her2/neu | 0-1-2 | 308 | (85.1%) |
| | 3 | 54 | (14.9%) |
| Hormonotherapy | N | 212 | (45.3%) |
| | Y | 256 | (54.7%) |
| Follow-up | median [IC95] | 71 mois [68-73] | |
| Metastasis | N | 364 | (72.2%) |
| | Y | 140 | (27.8%) |
| 5 years MFS (Metastasis | MFS [IC95] | 73.52 | [69.55-77.72] |
| Free Survival) | | | |
| Deaths from breast | N | 412 | (81.7%) |
| cancer | Y | 92 | (18.3%) |
| Specific Survival at 5 | SS [IC95] | 84.87 | [81.56-88.31] |
| years | | | |

Identification Set demography (IPC, Lyon, Total):

| Age | median | 52 | | 48 | | 51 | |
|---|---|---|---|---|---|---|---|
| menopausal | Y | 110 | (52%) | 67 | (61%) | 177 | (55%) |
| | N | 103 | (48%) | 40 | (36%) | 143 | (45%) |
| Tumour size | pT1 | 57 | (27%) | 17 | (16%) | 74 | (23%) |
| | pT2 | 115 | (54%) | 73 | (66%) | 188 | (58%) |
| | pT3 | 41 | (19%) | 20 | (18%) | 61 | (19%) |
| N | N− | 56 | (26%) | 11 | (10%) | 67 | (21%) |
| | N+ | 157 | (74%) | 99 | (90%) | 256 | (79%) |
| N. cat | N1 | 43 | (20%) | 12 | (11%) | 55 | (17%) |
| | N2 | 72 | (34%) | 60 | (55%) | 132 | (41%) |
| | N3 | 98 | (46%) | 41 | (34%) | 139 | (43%) |
| grade SBR | I | 29 | (14%) | 16 | (15%) | 45 | (14%) |
| | II | 99 | (46%) | 55 | (50%) | 154 | (48%) |
| | III | 82 | (38%) | 39 | (35%) | 121 | (38%) |
| RE (10%) | RE− | 80 | (43%) | 32 | (31%) | 112 | (38%) |
| | RE+ | 104 | (57%) | 76 | (69%) | 180 | (62%) |
| RP (10%) | RP− | 62 | (38%) | 30 | (29%) | 92 | (34%) |
| | RP+ | 100 | (62%) | 78 | (71%) | 178 | (56%) |
| RH (10%) | RH− | 46 | (24%) | 23 | (21%) | 69 | (23%) |
| | RH+ | 143 | (76%) | 86 | (79%) | 229 | (77%) |
| Her2/neu | 0-1-2 | 174 | (82%) | 19 | (17%) | 193 | (60%) |
| | 3 | 35 | (16%) | 1 | (<1%) | 36 | (11%) |
| | NA | 4 | (2%) | 90 | (78%) | 94 | (29%) |
| Hormono- | N | 77 | (36%) | 38 | (35%) | 105 | (33%) |
| therapy | Y | 136 | (64%) | 72 | (65%) | 208 | (67%) |
| Follow-up | median | 61 | | 84 | | 70 | |
| Metastasis | N | 163 | (77%) | 73 | (66%) | 236 | (70%) |
| | Y | 50 | (23%) | 37 | (34%) | 87 | (30%) |
| 5 year MFS | MFS | 77.5% | | 71.8% | | 75.6% | |
| Deaths from | N | 172 | (81%) | 85 | (77%) | 257 | (80%) |
| breast cancer | Y | 41 | (19%) | 25 | (23%) | 66 | (20%) |

| | | | | |
|---|---|---|---|---|
| Specific | SS | 81.7% | 82.7% | 82% |
| Survival at | | | | |
| 5 years | | | | |

Validation Set demography (PACS01, Bordeaux, total):

| Age | median | 50 | | 44 | | 49 | |
|---|---|---|---|---|---|---|---|
| | (min-max) | | | | | | |
| menopausal | Y | 60 | (37%) | 1 | (6%) | 61 | (34%) |
| | N | 104 | (63%) | 15 | (88%) | 119 | (66%) |
| Tumour size | pT1 | 27 | (16%) | 9 | (53%) | 37 | (20%) |
| | pT2 | 116 | (71%) | 8 | (47%) | 125 | (69%) |
| | pT3 | 16 | (10%) | 0 | (0%) | 16 | (9%) |
| N | N− | 0 | (0%) | 0 | (0%) | 0 | (0%) |
| | N+ | 164 | (100%) | 17 | (100%) | 181 | (0%) |
| N. cat | N1 | 0 | (0%) | 0 | (0%) | 0 | (0%) |
| | N2 | 80 | (49%) | 14 | (82%) | 94 | (52%) |
| | N3 | 84 | (51%) | 3 | (18%) | 87 | (48%) |
| grade SBR | I | 24 | (15%) | 2 | (12%) | 26 | (14%) |
| | II | 60 | (37%) | 7 | (41%) | 67 | (37%) |
| | III | 75 | (46%) | 8 | (47%) | 83 | (46%) |
| RE (10%) | RE− | 121 | (74%) | 5 | (29%) | 126 | (70%) |
| | RE+ | 31 | (19%) | 12 | (71%) | 43 | (24%) |
| RP (10%) | RP− | 54 | (33%) | 4 | (24%) | 58 | (32%) |
| | RP+ | 110 | (67%) | 13 | (76%) | 123 | (68%) |
| RH (10%) | RH− | 33 | (20%) | 3 | (18%) | 37 | (20%) |
| | RH+ | 131 | (80%) | 14 | (82%) | 145 | (80%) |
| Her2/neu | 0-1-2 | 113 | (69%) | 13 | (76%) | 126 | (70%) |
| | 3 | 15 | (9%) | 4 | (24%) | 19 | (10%) |
| | NA | 36 | (22%) | 0 | (0%) | 36 | (20%) |
| Hormono- | N | 53 | (32%) | 14 | (82%) | 67 | (37%) |
| therapy | Y | 76 | (46%) | 3 | (18%) | 79 | (44%) |
| | NA | 36 | (22%) | 0 | (0%) | 36 | (19%) |
| Follow-up | median | 59 | | 123 | | 59 | |
| Metastasis | N | 127 | (77%) | 12 | (71%) | 139 | (77%) |
| | Y | 37 | (23%) | 5 | (29%) | 42 | (23%) |
| 5 y MFS | MFS | 78.6% | | 70.6% | | 77.8% | |
| Deaths from | N | 140 | (85%) | 12 | (71%) | 152 | (84%) |
| breast cancer | Y | 24 | (15%) | 5 | (29%) | 29 | (16%) |

| | | | | |
|---|---|---|---|---|
| Specific | SS | 85.4% | 70.6% | 84% |
| Survival at | | | | |
| 5 years | | | | |

### 3) Method for Gene Profiling:

Radio-labeled [A³³P]-dCTP cDNA probes are obtained by reverse transcription from 3 μg of total RNA. Probes are then hybridised on IPSOGEN's 10K DiscoveryChip™, consisting of nylon membranes containing 9600 spotted cDNA (Discovery™ platform).

Following hybridization, membranes are washed and exposed to phosphor-imaging plates, then scanned with a Fuji-BAS 5000 machine. Signal intensities are quantified using the Fuji ArrayGauge v1.2 program, and the resulting raw data are analysed.

### 4) Analysis:

### 4-1: Normalisation and Filtering:

Raw data are exported from Ipsogen database. Spots for which spotted DNA amount is too low are invalidated from further analysis. Data are then normalized as compared to a reference sample using a non-linear rank based method (Sabatti et al., 2002). Normalized data are then filtered to eliminate low intensity genes, for which expression level is comparable to non-specific signal and the measure highly uncertain.

Data quality controls are performed based on hierarchical clustering grouping samples and genes according to their profile similarity. Biological pertinence of samples and genes clusters insures good quality data and allow for further analysis.

Since we analysed several samples series we performed a supplementary data normalization to insure inter-series comparability. Comparability was checked by hierarchical clustering.

### 4-2: Phenotypic Signatures Identification:

We performed supervised analysis using MaxT method available on Bioconductor (Ge, Dudoit & Speed, 2003) for several phenotypic markers: ER, PR, HER2/Neu, MIB/KI67, EGFR. The five markers were all measured by standard immunohistochemistry (IHC).

Supervised analyses were performed on a 159 samples identification set for ER, PR, HER2/Neu and EGFR markers, and on a 114 samples identification set for MIB/KI67. Each identified signature was then validated on one to four independent datasets.

Validation consisted in status prediction for independent samples using the LPS method (Linear Predictor Score) (Wright et al., PNAS, 2003, vol. 100, no. 17, 9991-9996). Prediction of all independent samples allowed for sensitivity and specificity evaluation for each identified signature.

### 4-3: Metagenes Calculation:

We considered as a metagene a group of genes for which expression variation (but not necessarily expression level) across tumors is correlated. The assumption is that the error made on the measurement of expression level from a single gene is highly reduced when considering several genes. So even in the case that an individual gene is poorly measured, its contribution in the metagene value is weighted by the number of genes considered and the final value for the metagene is lowly affected.

Metagenes were calculated from both supervised and unsupervised data.

Metagenes from phenotypic signatures: Phenotypic signatures correspond to genes correlated with a given phenotypic marker assessed by current standards such as immunohistochemistry (IHC) or FISH. A gene is considered correlated by a modified t test (MaxT method) which tests the significance of differential expression with a 5% risk. Each phenotypic signature is composed of two gene subsets, which expression levels are anti-correlated. One group of gene is overexpressed in a group of tumours (for example ER+ tumours) while the other group is underexpressed in the same group of tumours. Although expression variation is correlated across samples, expression levels may vary between genes, then leading to non robust average expression. It is assumed that even if expression levels vary, differential expression according to a reference sample belongs to the same dynamic range for all genes, allowing average calculation. For each tumour, each gene measure is divided by the expression level of the gene in a reference sample (log ratio) and the corresponding metagene is the average of those log ratios.

Each signature allowed the calculation of two anti-correlated metagenes. For instance, ER signature gives 2 metagenes, underER (genes under expressed in ER+ tumours) and overER (genes over expressed in ER+ tumours).

Metagenes from unsupervised analyses: we also defined metagenes as groups of genes with correlated expression variation across samples based on hierarchical clustering on a 468 samples set. A group of genes was retained if it contained at least 5 genes and had a node correlation coefficient higher than 0.5. Groups of genes that corresponded to previously identified metagenes by supervised analysis were not further considered. Metagenes were obtained as the mean of the log ratios of the genes contained in a given group.

### 4-4: Biostatistics

Since we failed to identify any robust gene signature based on classical supervised analysis for the metastasis, it seems that obviously a single set of correlated genes is not able to predict metastasis.

The biostatistic approach was then based on survival analysis, and the objective was, instead of separating metastasis from non metastasis patients, to identify two groups of patients with significantly different outcome. The event considered is the metastasis without considering any previous event such as local relapse.

Model calculation: We used the Cox regression to identify a combination of metagenes able to add prognostic information to already existing prognostic factors, such as SBR grade, tumour size, or lymph node involvement. Cox proportional hazard ratio analysis consists in the calculation of a likelihood function, which gives for a patient the probability to observe the event at a given time (death, metastasis), knowing that he survived until this time. The likelihood function is independent of time, and takes into account a “baseline” risk which is common to every patient, and the risk which is associated to different explanatory variables (which values differ between patients). The baseline risk function is unknown and eliminated as far as ratios between patients are considered. Then, the log-likelihood is defined as a linear function of explanatory variables, each one being appropriately weighted by a given coefficient. The coefficients are estimated by the algorithm to maximize the log-likelihood function.

For this, we use a forward stepwise approach to select the most significant metagenes, the threshold p-value being fixed to 10%. To obtain a model dependant on metagenes information and not influenced by already known clinical parameters, the analysis was stratified on the clinical parameters SBR grade, tumour size and lymph node synthesized in a single parameter, the NPI (Nottingham Prognostic Index). Moreover, since the identification set was composed of patients originating from different anti-cancer centers, we also stratified the analysis on the center of origin.

Once a combination of metagenes was obtained we calculated for each patient a score based on the linear combination of the metagenes values weighted by the coefficient calculated by the algorithm for each metagene. The exponential value of the coefficient corresponds to the hazard ratio associated to the metagene. For each parameter estimation, the algorithm gives the 95% confidence interval. Hence any combination of values comprised in the confidence intervals can be used to separate patients into significantly different prognostic groups.

Prognostic groups determination: The distribution of the scores in the identification set was used to determine the most significant cut-off to separate patients into two groups of different outcome. We tested three thresholds, 1^{st}, 2^{nd}, and 3^{rd} quartile, and performed in each case the logrank test to compare the two groups of patients. We used a step by step approach to define the optimized threshold, testing all score values as a potential threshold.

The cut-off was the one for which the p value associated to the log rank test was the most significant.

Validation on an independent validation set: for each patient of the validation set, we calculated the score and separated the patients into two prognostic groups using the coefficients and the threshold determined on the identification set. The score was calculated without considering the outcome (DFS-Disease Free Survival) of individual patients.

The validation was appreciated by the p value of the log rank test, which has to be <5% to consider the model validated.

We verified that the identified model effectively added relevant information as compared to standard parameters by performing multivariate Cox analyses which integrate clinical parameters and the model.

Sample prediction: For any new sample to be predicted raw data are normalized according to the reference sample previously defined and metagenes are calculated. The formula calculated on the identification set is then applied to the new sample, allowing the attribution of a specific score to each sample. The score is compared to the threshold optimized from the identification procedure and the patient is declared to belong to the good prognosis group if its score is lower or equal to the threshold and to the poor prognosis group if its score is higher than the threshold.

### 5) Results:

### 5-1: Metagene Selection

We started from 9 metagenes calculated from supervised analyses, and 17 metagenes from unsupervised analysis.

A first analysis based on the correlation between metagenes and robustness reduced the potential candidates to 19 metagenes, 7 from supervised analysis and 12 from unsupervised analysis.

### 5-2: Univariate Analysis

Each metagene was first tested in a univariate Cox analysis, and none of them could be found significant alone as shown in the following table.

| | Parameter | | |
|---|---|---|---|
| Variable | Estimate | Hazard Ratio | p value |
| underER | 0.468 | 1.597 | 0.59 |
| underPR | −0.474 | 0.622 | 0.32 |
| underEGFR | −1.132 | 0.322 | 0.06 |
| overEGFR | −0.261 | 0.771 | 0.59 |
| underMIB | −0.951 | 0.387 | 0.18 |
| overMIB | 0.927 | 2.528 | 0.37 |
| overERBB2 | 0.089 | 1.094 | 0.88 |
| MG48 | −0.398 | 0.672 | 0.46 |
| MG187 | −0.453 | 0.636 | 0.38 |
| MG66 | −0.423 | 0.655 | 0.40 |
| MG27 | 0.193 | 1.21 | 0.65 |
| MG51 | −0.182 | 0.834 | 0.75 |
| MG141 | −0.076 | 0.927 | 0.90 |
| MG144 | −0.256 | 0.774 | 0.70 |
| MG171 | 0.131 | 1.14 | 0.82 |
| MG240 | −0.304 | 0.738 | 0.55 |
| MG310 | 0.271 | 1.31 | 0.61 |
| MG448 | −1.03 | 0.358 | 0.10 |
| MG1001 | −0.34 | 0.712 | 0.31 |

### 5-3: Description of Selected Metagenes and Combination Thereof

Multivariate Cox analyses allowed identification of significant metagenes and combinations thereof associated with prognosis. The constituents of the selected metagenes and these combinations are described hereafter.

### Example 2

### Identification of a First Metagene Combination

The Cox analysis using forward stepwise procedure identified the three following significant metagenes (underER, underPR and underEGFR) associated with good or poor prognosis.

TABLE I
(Metagene UnderER)

| | | | | | Reduced | Reduced |
|---|---|---|---|---|---|---|
| | | | | | metagene | metagene |
| Gene | Unigene Cluster | Regulation | P value | Ref. Seq | 27 | 20 |
| ITGB3 | ughs.218040:186 | − | 0.00001 | SEQ ID | + | + |
| integrin, beta 3 (platelet | | | | No: 374 | | |
| glycoprotein iiia, | | | | (nm_000212) | | |
| antigen cd61) | | | | | | |
| PADI2 | ughs.33455:186 | − | 0.00001 | SEQ ID | + | + |
| peptidyl arginine | | | | No: 1027 | | |
| deiminase, type ii | | | | (nm_007365) | | |
| SOD2 | ughs.487046:186 | − | 0.00001 | SEQ ID | + | + |
| superoxide dismutase | | | | No: 598 | | |
| 2, mitochondrial | | | | (nm_000636) | | |
| FLJ13154 | ughs.408702:186 | − | 0.00003 | SEQ ID | − | − |
| hypothetical protein | | | | No: 717 | | |
| flj13154 | | | | (nm_024598) | | |
| HDAC2 | ughs.3352:186 | − | 0.00004 | SEQ ID | + | + |
| histone deacetylase 2 | | | | No: 573 | | |
| | | | | (nm_001527) | | |
| SLAC2-B | N_A | − | 0.00006 | SEQ ID | + | + |
| | | | | No: 83 | | |
| | | | | (nm_015065) | | |
| S100A8 | ughs.416073:186 | − | 0.00006 | SEQ ID | + | + |
| s100 calcium binding | | | | No: 12 | | |
| protein a8 (calgranulina) | | | | (nm_002964) | | |
| GSTP1 | ughs.523836:186 | − | 0.00006 | SEQ ID | + | + |
| glutathione s- | | | | No: 405 | | |
| transferase pi | | | | (nm_000852) | | |
| LCN2 | ughs.204238:186 | − | 0.00012 | SEQ ID | + | + |
| lipocalin 2 (oncogene | | | | No: 856 | | |
| 24p3) | | | | (nm_005564) | | |
| MYBL2 | ughs.179718:186 | − | 0.00013 | SEQ ID | + | − |
| v-myb myeloblastosis | | | | No: 384 | | |
| viral oncogene homolog | | | | (nm_002466) | | |
| (avian)-like 2 | | | | | | |
| PFKP | ughs.26010:186 | − | 0.00081 | SEQ ID | + | + |
| phosphofructokinase, | | | | No: 167 | | |
| platelet | | | | (nm_002627) | | |
| STK6 | ughs.250822:186 | − | 0.00134 | SEQ ID | + | + |
| serine/threonine kinase 6 | | | | No: 51 | | |
| | | | | (nm_198433) | | |
| GPR125 | ughs.99195:186 | − | 0.00153 | SEQ ID | − | − |
| g protein-coupled | | | | No: 999 | | |
| receptor 125 | | | | (nm_145290) | | |
| DSCR1 | ughs.282326:186 | − | 0.00206 | SEQ ID | − | − |
| down syndrome critical | | | | No: 979 | | |
| region gene 1 | | | | (nm_004414) | | |
| FAT | ughs.481371:186 | − | 0.0023 | SEQ ID No: 2 | + | − |
| fat tumor suppressor | | | | (nm_005245) | | |
| homolog 1 (drosophila) | | | | | | |
| VGLL1 | N_A | − | 0.00247 | SEQ ID | + | + |
| vestigial like 1 | | | | No: 98 | | |
| (drosophila) | | | | (nm_016267) | | |
| MMP7 | ughs.2256:186 | − | 0.00264 | SEQ ID | + | + |
| matrix | | | | No: 751 | | |
| metalloproteinase 7 | | | | (nm_002423) | | |
| (matrilysin, uterine) | | | | | | |
| ENO1 | ughs.517145:186 | − | 0.00348 | SEQ ID | + | + |
| enolase 1, (alpha) | | | | No: 696 | | |
| | | | | (nm_001428) | | |
| cdna clone | ughs.175285:186 | − | 0.00429 | SEQ ID | + | − |
| image:4831215 | | | | No: 1050 | | |
| | | | | (BC034638) | | |
| SCP2 | ughs.476365:186 | − | 0.00469 | SEQ ID | − | − |
| sterol carrier protein 2 | | | | No: 488 | | |
| | | | | (nm_002979) | | |
| CEBPB | ughs.517106:186 | − | 0.00507 | SEQ ID | + | + |
| ccaat/enhancer binding | | | | No: 262 | | |
| protein (c/ebp), beta | | | | (nm_005194) | | |
| TGM1 | ughs.508950:186 | − | 0.00695 | SEQ ID | + | + |
| transglutaminase 1 (k | | | | No: 1020 | | |
| polypeptide epidermal | | | | (nm_000359) | | |
| type i, protein- | | | | | | |
| glutamine-gamma- | | | | | | |
| glutamyltransferase) | | | | | | |
| | N_A | − | 0.00764 | SEQ ID | − | − |
| | | | | No: 1106 | | |
| | | | | (BC015969) | | |
| GGH | ughs.78619:186 | − | 0.00881 | SEQ ID | + | − |
| gamma-glutamyl | | | | No: 952 | | |
| hydrolase (conjugase, | | | | (nm_003878) | | |
| folylpolygammaglutamyl | | | | | | |
| hydrolase) | | | | | | |
| GSTA4 | ughs.485557:186 | − | 0.00995 | SEQ ID | − | − |
| glutathione s- | | | | No: 675 | | |
| transferase a4 | | | | (nm_001512) | | |
| FN5 | ughs.438064:186 | − | 0.0109 | SEQ ID | − | − |
| b-cell cll/lymphoma 7b | | | | No: 289 | | |
| | | | | (nm_020179) | | |
| CCNB2 | ughs.194698:186 | − | 0.01221 | SEQ ID | − | − |
| glutamate | | | | No: 553 | | |
| decarboxylase 1 (gad | | | | (nm_004701) | | |
| 1) | | | | | | |
| CTSC | ughs.128065:186 | − | 0.01501 | SEQ ID | + | + |
| cathepsin c | | | | No: 579 | | |
| | | | | (nm_001814) | | |
| PBEF1 | ughs.489615:186 | − | 0.01621 | SEQ ID | + | + |
| pre-b-cell colony | | | | No: 760 | | |
| enhancing factor 1 | | | | (nm_005746) | | |
| S100A6 | ughs.275243:186 | − | 0.01719 | SEQ ID | + | + |
| s100 calcium binding | | | | No: 805 | | |
| protein a6 (calcyclin) | | | | (nm_014624) | | |
| RDX | ughs.263671:186 | − | 0.01753 | SEQ ID | + | − |
| radixin | | | | No: 361 | | |
| | | | | (nm_002906) | | |
| GPR126 | ughs.318894:186 | − | 0.01886 | SEQ ID | − | − |
| g protein-coupled | | | | No: 448 | | |
| receptor 126 | | | | (nm_198569) | | |
| MMP15 | ughs.80343:186 | − | 0.0274 | SEQ ID | − | − |
| matrix | | | | No: 170 | | |
| metalloproteinase 15 | | | | (nm_002428) | | |
| (membrane-inserted) | | | | | | |
| KLK6 | ughs.79361:186 | − | 0.02892 | SEQ ID | + | + |
| kallikrein 6 (neurosin, | | | | No: 878 | | |
| zyme) | | | | (nm_002774) | | |
| | N_A | − | 0.0351 | SEQ ID | − | − |
| | | | | No: 1117 | | |
| BOK | ughs.293753:186 | − | 0.03747 | SEQ ID | + | + |
| bcl2-related ovarian | | | | No: 612 | | |
| killer | | | | (nm_032515) | | |
| CDKL5 | ughs.435570:186 | − | 0.03754 | SEQ ID | − | − |
| cyclin-dependent | | | | No: 540 | | |
| kinase-like 5 | | | | (nm_003159) | | |
| CSTB | ughs.695:186 | − | 0.0382 | SEQ ID | − | − |
| cystatin b (stefin b) | | | | No: 823 | | |
| | | | | (nm_000100) | | |
| LOC151194 | ughs.552610:186 | − | 0.03884 | SEQ ID | − | − |
| similar to hepatocellular | | | | No: 131 | | |
| carcinoma-associated | | | | (nm_145280) | | |
| antigen hca557b | | | | | | |
| NFIB | ughs.370359:186 | − | 0.03949 | SEQ ID | − | − |
| nuclear factor i/b | | | | No: 705 | | |
| | | | | (nm_005596) | | |
| LAD1 | ughs.519035:186 | − | 0.04184 | SEQ ID | + | − |
| ladinin 1 | | | | No: 31 | | |
| | | | | (nm_005558) | | |
| MGC11271 | ughs.143288:18 | − | 0.04312 | SEQ ID | + | − |
| hypothetical protein 6 | | | | No: 199 | | |
| mgc11271 | | | | (nm_024323) | | |

TABLE II
(Metagene Under PR)

| | | | | | Reduced | Reduced |
|---|---|---|---|---|---|---|
| | | | | | Metagene | Metagene |
| Gene | Unigene Cluster | Regulation | P value | Ref. Seq | 35 | 6 |
| SOD2 | ughs.487046:186 | − | 0.00001 | SEQ ID | − | − |
| superoxide dismutase | | | | No: 598 | | |
| 2, mitochondrial | | | | (nm_000636) | | |
| IGHG1 | ughs.510635:186 | − | 0.00001 | SEQ ID | − | − |
| immunoglobulin heavy | | | | No: 1122 | | |
| constant gamma 1 | | | | | | |
| (g1m marker) | | | | | | |
| KDR | ughs.479756:186 | − | 0.00011 | SEQ ID | + | + |
| kinase insert domain | | | | No: 364 | | |
| receptor (a type iii | | | | (nm_002253) | | |
| receptor tyrosine | | | | | | |
| kinase) | | | | | | |
| KLF1 | ughs.37860:186 | − | 0.00014 | SEQ ID | + | − |
| kruppel-like factor 1 | | | | No: 387 | | |
| (erythroid) | | | | (nm_006563) | | |
| CASP9 | ughs.329502:186 | − | 0.00016 | SEQ ID | + | + |
| caspase 9, apoptosis- | | | | No: 34 | | |
| related cysteine | | | | (nm_001229) | | |
| protease | | | | | | |
| BCL2 | ughs.150749:186 | − | 0.00018 | SEQ ID | + | + |
| b-cell oil/lymphoma 2 | | | | No: 657 | | |
| | | | | (nm_000633) | | |
| MYBL2 | ughs.179718:186 | − | 0.00025 | SEQ ID | − | − |
| v-myb myeloblastosis | | | | No: 384 | | |
| viral oncogene | | | | (nm_002466) | | |
| homolog (avian)-like 2 | | | | | | |
| ADAM10 | ughs.172028:186 | − | 0.00031 | SEQ ID | − | − |
| a disintegrin and | | | | No: 451 | | |
| metalloproteinase | | | | (nm_001110) | | |
| domain 10 | | | | | | |
| GPR125 | ughs.99195:186 | − | 0.00032 | SEQ ID | − | − |
| g protein-coupled | | | | No: 999 | | |
| receptor 125 | | | | (nm_145290) | | |
| | ughs.26192:186 | − | 0.00049 | SEQ ID | + | − |
| | | | | No: 1056 | | |
| | | | | (AK126297) | | |
| TGFBR3 | ughs.482390:186 | − | 0.00061 | SEQ ID | + | − |
| transforming growth | | | | No: 15 | | |
| factor, beta receptor iii | | | | (nm_003243) | | |
| (betaglycan, 300 kda) | | | | | | |
| LOC91316 | ughs.407693:186; | − | 0.00072 | SEQ ID | − | − |
| similar to bk246h3.1 | ughs.148656:186 | | | No: 1090 | | |
| (immunoglobulin | | | | (AK125808) | | |
| lambda-like | | | | | | |
| polypeptide 1, pre-b- | | | | | | |
| cell specific) | | | | | | |
| | ughs.416139:186 | − | 0.00074 | SEQ ID | + | − |
| | | | | No: 1120 | | |
| S100A8 | ughs.416073:186 | − | 0.00079 | SEQ ID | − | − |
| s100 calcium binding | | | | No: 12 | | |
| protein a8 (calgranulina) | | | | (nm_002964) | | |
| PIM2 | ughs.496096:186 | − | 0.00088 | SEQ ID | − | − |
| pim-2 oncogene | | | | No: 743 | | |
| | | | | (nm_006875) | | |
| TP53 | ughs.408312:186 | − | 0.00104 | SEQ ID | + | − |
| tumor protein p53 (li- | | | | No: 414 | | |
| fraumeni syndrome) | | | | (nm_000546) | | |
| ITGB3 | ughs.218040:186 | − | 0.00118 | SEQ ID | + | − |
| integrin, beta 3 | | | | No: 374 | | |
| (platelet glycoprotein | | | | (nm_000212) | | |
| iiia, antigen cd61) | | | | | | |
| LAMB1 | ughs.489646:186 | − | 0.00118 | SEQ ID | + | − |
| laminin, beta 1 | | | | No: 711 | | |
| | | | | (nm_002291) | | |
| SILV | ughs.95972:186 | − | 0.00118 | SEQ ID | + | − |
| silver homolog | | | | No: 663 | | |
| (mouse) | | | | (nm_006928) | | |
| cdna flj42596 fis, clone | ughs.113271:186 | − | 0.00121 | SEQ ID | − | − |
| brace3010283 | | | | No: 1102 | | |
| | | | | (AK124587) | | |
| PIGR | ughs.497589:186 | − | 0.00123 | SEQ ID | + | − |
| polymeric | | | | No: 237 | | |
| immunoglobulin | | | | (nm_002644) | | |
| receptor | | | | | | |
| CSH1 | ughs.347963:186 | − | 0.00161 | SEQ ID | + | − |
| chorionic | | | | No: 60 | | |
| somatomammotropin | | | | (nm_022640) | | |
| hormone 1 (placental | | | | | | |
| lactogen) | | | | | | |
| RDX | ughs.263671:186 | − | 0.00176 | SEQ ID | − | − |
| radixin | | | | No: 361 | | |
| | | | | (nm_002906) | | |
| ETF1/FLT1 | ughs.483494:186; | − | 0.0019 | SEQ ID | + | − |
| eukaryotic translation | ughs.507621:186 | | | No: 119 | | |
| termination factor | | | | (nm_004730) | | |
| 1/fms-related tyrosine | | | | or | | |
| kinase 1 | | | | SEQ ID | | |
| | | | | No: 1109 | | |
| | | | | (NM_002019) | | |
| PFKP | ughs.26010:186 | − | 0.00193 | SEQ ID | − | − |
| phosphofructokinase, | | | | No: 167 | | |
| platelet | | | | (nm_002627) | | |
| CXORF38 | ughs.495961:186 | − | 0.002 | SEQ ID | + | + |
| chromosome x open | | | | No: 339 | | |
| reading frame 38 | | | | (nm_144970) | | |
| MGC15606 | ughs.130195:186 | − | 0.00207 | SEQ ID | − | − |
| family with sequence | | | | No: 333 | | |
| similarity 55, member c | | | | (nm_145037) | | |
| SLAC2-B | N_A | − | 0.00236 | SEQ ID | − | − |
| slac2-b | | | | No: 83 | | |
| | | | | (nm_015065) | | |
| FLJ10986 | ughs.444301:186; | − | 0.00261 | SEQ ID | + | − |
| hypothetical protein | ughs.439112:186 | | | No: 330 | | |
| flj10986 | | | | (nm_018291) | | |
| SERPINB1 | ughs.381167:186 | − | 0.00368 | SEQ ID | + | − |
| serine (or cysteine) | | | | No: 1024 | | |
| proteinase inhibitor, | | | | (nm_030666) | | |
| clade b (ovalbumin), | | | | | | |
| member 1 | | | | | | |
| RPS6KA3 | ughs.445387:186 | − | 0.00482 | SEQ ID | + | + |
| ribosomal protein s6 | | | | No: 229 | | |
| kinase, 90 kda, | | | | (nm_004586) | | |
| polypeptide 3 | | | | | | |
| GATA6 | ughs.514746:186 | − | 0.00491 | SEQ ID | + | − |
| gata binding protein 6 | | | | No: 925 | | |
| | | | | (nm_005257) | | |
| MTIF2 | ughs.149894:186 | − | 0.00535 | SEQ ID | − | − |
| mitochondrial | | | | No: 788 | | |
| translational initiation | | | | (nm_001005369) | | |
| factor 2 | | | | | | |
| | N_A | − | 0.00572 | SEQ ID | + | − |
| | | | | No: 1104 | | |
| | | | | (AK128524) | | |
| | N_A | − | 0.00635 | SEQ ID | + | − |
| | | | | No: 1103 | | |
| | | | | (BX108410) | | |
| IFNGR1 | ughs.520414:186 | − | 0.00656 | SEQ ID | + | − |
| interferon gamma | | | | No: 66 | | |
| receptor 1 | | | | (nm_000416) | | |
| EBF | ughs.308048:186 | − | 0.00665 | SEQ ID | − | − |
| early b-cell factor | | | | No: 1030 | | |
| | | | | (nm_024007) | | |
| | N_A | − | 0.00729 | SEQ ID | + | + |
| | | | | No: 1119 | | |
| p66alpha | ughs.551742:186 | − | 0.00741 | SEQ ID | + | − |
| GATA zinc finger | | | | No: 1068 | | |
| domain containing 2A | | | | (AK024670) | | |
| (p66alpha) | | | | | | |
| FKBP1A | ughs.471933:186 | − | 0.00885 | SEQ ID | − | − |
| fk506 binding protein | | | | No: 241 | | |
| 1a, 12 kda | | | | (nm_000801) | | |
| SNAPC3 | ughs.546299:186 | − | 0.00887 | SEQ ID | − | − |
| small nuclear rna | | | | No: 398 | | |
| activating complex, | | | | (nm_003084) | | |
| polypeptide 3, 50 kda | | | | | | |
| IL2RB | ughs.474787:186; | − | 0.0097 | SEQ ID | + | − |
| interleukin 2 receptor, | ughs.555488:186 | | | No: 74 | | |
| beta | | | | (nm_000878) | | |
| Homo sapiens mRNA | ughs.535157:186 | − | 0.00973 | SEQ ID | − | − |
| for FLJ00204 protein | | | | No: 1087 | | |
| | | | | (AK074131) | | |
| ETV4 | ughs.434059:186 | − | 0.01003 | SEQ ID | − | − |
| ets variant gene 4 | | | | No: 955 | | |
| (e1a enhancer binding | | | | (nm_001986) | | |
| protein, e1af) | | | | | | |
| IL1R2 | ughs.25333:186 | − | 0.01009 | SEQ ID | − | − |
| interleukin 1 receptor, | | | | No: 71 | | |
| type ii | | | | (nm_004633) | | |
| IGHG1 | ughs.510635:186 | − | 0.01039 | SEQ ID | − | − |
| immunoglobulin heavy | | | | No: 1105 | | |
| constant gamma 1 | | | | (BC072392) | | |
| (g1m marker) | | | | | | |
| LCN2 | ughs.204238:186 | − | 0.01068 | SEQ ID | − | − |
| lipocalin 2 (oncogene | | | | No: 856 | | |
| 24p3) | | | | (nm_005564) | | |
| CMRF35 | ughs.2605:186 | − | 0.01119 | SEQ ID | + | − |
| cd300c antigen | | | | No: 231 | | |
| | | | | (nm_006678) | | |
| CXCL1 | ughs.789:186 | − | 0.01174 | SEQ ID | + | − |
| chemokine (c-x-c | | | | No: 593 | | |
| motif) ligand 1 | | | | (nm_001511) | | |
| (melanoma growth | | | | | | |
| stimulating activity, | | | | | | |
| alpha) | | | | | | |
| MYBL2 | ughs.179718:186 | − | 0.0122 | SEQ ID | + | − |
| v-myb myeloblastosis | | | | No: 384 | | |
| viral oncogene | | | | (nm_002466) | | |
| homolog (avian)-like 2 | | | | | | |
| SLAMF8 | ughs.438683:186 | − | 0.01309 | SEQ ID | − | − |
| slam family member 8 | | | | No: 519 | | |
| | | | | (nm_020125) | | |
| CTSC | ughs.128065:186 | − | 0.016 | SEQ ID | − | − |
| cathepsin c | | | | No: 579 | | |
| | | | | (nm_001814) | | |
| ENPP2 | ughs.190977:186 | − | 0.0205 | SEQ ID | + | − |
| ectonucleotide | | | | No: 1039 | | |
| pyrophosphatase/phosphodiesterase 2 | | | | (nm_006209) | | |
| (autotaxin) | | | | | | |
| LAD1 | ughs.519035:186 | − | 0.02102 | SEQ ID | − | − |
| ladinin 1 | | | | No: 31 | | |
| | | | | (nm_005558) | | |
| RABL3 | ughs.444360:186; | − | 0.02205 | SEQ ID | + | − |
| rab, member of ras | ughs.548087:186 | | | No: 327 | | |
| oncogene family-like 3 | | | | (nm_173825) | | |
| HDAC2 | ughs.3352:186 | − | 0.02428 | SEQ ID | − | − |
| histone deacetylase 2 | | | | No: 573 | | |
| | | | | (nm_001527) | | |
| VGLL1 | N_A | − | 0.02447 | SEQ ID | − | − |
| vestigial like 1 | | | | No: 98 | | |
| (drosophila) | | | | (nm_016267) | | |
| npc-a-5 | ughs.510543:186 | − | 0.02592 | SEQ ID | − | − |
| nasopharyngeal | | | | No: 1059 | | |
| carcinoma-associated | | | | (AK091113) | | |
| antigen npc-a-5 | | | | | | |
| CDK4 | ughs.95577:186 | − | 0.02615 | SEQ ID | + | − |
| cyclin-dependent | | | | No: 886 | | |
| kinase 4 | | | | (nm_000075) | | |
| ABCC5 | ughs.368563:186 | − | 0.02624 | SEQ ID | + | − |
| atp-binding cassette, | | | | No: 1032 | | |
| sub-family c (cftr/mrp), | | | | (nm_005688) | | |
| member 5 | | | | | | |
| MGC9913 | ughs.23133:186 | − | 0.02709 | SEQ ID | − | − |
| hypothetical protein | | | | No: 1091 | | |
| mgc9913 | | | | (XM_378178) | | |
| FUT8 | ughs.118722:186 | − | 0.02833 | SEQ ID | − | − |
| fucosyltransferase 8 | | | | No: 233 | | |
| (alpha (1,6) | | | | (nm_178155) | | |
| fucosyltransferase) | | | | | | |
| SFRP1 | ughs.213424:186 | − | 0.03011 | SEQ ID | − | − |
| secreted frizzled- | | | | No: 938 | | |
| related protein 1 | | | | (nm_003012) | | |
| ARPC2 | ughs.529303:186 | − | 0.03237 | SEQ ID | + | − |
| actin related protein | | | | No: 264 | | |
| 2/3 complex, subunit | | | | (nm_152862) | | |
| 2, 34 kda | | | | | | |
| LILRB2 | ughs.534386:186 | − | 0.03294 | SEQ ID | − | − |
| leukocyte | | | | No: 546 | | |
| immunoglobulin-like | | | | (nm_005874) | | |
| receptor, subfamily b | | | | | | |
| (with tm and itim | | | | | | |
| domains), member 2 | | | | | | |
| IGKC | ughs.449621:186; | − | 0.03458 | SEQ ID | − | − |
| immunoglobulin kappa | ughs.546620:186 | | | No: 1099 | | |
| constant | | | | (BC066343) | | |
| SN | ughs.31869:186 | − | 0.03771 | SEQ ID | + | − |
| sialoadhesin | | | | No: 1037 | | |
| | | | | (nm_023068) | | |
| C1ORF38 | ughs.10649:186 | − | 0.03783 | SEQ ID | − | − |
| chromosome 1 open | | | | No: 550 | | |
| reading frame 38 | | | | (nm_004848) | | |
| PADI2 | ughs.33455:186 | − | 0.0418 | SEQ ID | − | − |
| peptidyl arginine | | | | No: 1027 | | |
| deiminase, type ii | | | | (nm_007365) | | |
| MONDOA | ughs.437153:186 | − | 0.04548 | SEQ ID | + | − |
| mix interactor | | | | No: 1005 | | |
| | | | | (nm_014938) | | |
| TAP1 | ughs.352018:186; | − | 0.04583 | SEQ ID | − | − |
| transporter 1, atp- | ughs.552165:186 | | | No: 820 | | |
| binding cassette, sub- | | | | (nm_000593) | | |
| family b (mdr/tap) | | | | | | |
| CYP2D6 | ughs.534311:186 | − | 0.04704 | SEQ ID | − | − |
| cytochrome p450, | | | | No: 370 | | |
| family 2, subfamily d, | | | | (nm_000106) | | |
| polypeptide 6 | | | | | | |

TABLE III
(Metagene UnderEGFR)

| | | | | | Reduced | Reduced |
|---|---|---|---|---|---|---|
| | | | | | Metagene | Metagene |
| Gene | Unigene Cluster | Regulation | P value | Ref. Seq | 34 | 22 |
| LOC255743: | N_A | − | 0.00001 | SEQ ID | + | + |
| Nephronectin | | | | No: 1071 | | |
| | | | | (NM_001033047) | | |
| LU | ughs.155048:186 | − | 0.00001 | SEQ ID | + | + |
| lutheran blood group | | | | No: 254 | | |
| (auberger b antigen | | | | (nm_005581) | | |
| included) | | | | | | |
| TFF1 | ughs.162807:186 | − | 0.00001 | SEQ ID No: 6 | + | + |
| trefoil factor 1 (breast | | | | (nm_003225) | | |
| cancer, estrogen- | | | | | | |
| inducible sequence | | | | | | |
| expressed in) | | | | | | |
| ESR1 | ughs.208124:186 | − | 0.00001 | SEQ ID | + | + |
| estrogen receptor 1 | | | | No: 883 | | |
| | | | | (nm_000125) | | |
| XBP1 | ughs.437638:186 | − | 0.00001 | SEQ ID | + | + |
| x-box binding protein 1 | | | | No: 543 | | |
| | | | | (nm_005080) | | |
| SCUBE2 | ughs.523468:186 | − | 0.00001 | SEQ ID | + | + |
| signal peptide, cub | | | | No: 681 | | |
| domain, egf-like 2 | | | | (nm_020974) | | |
| GATA3 | ughs.524134:186 | − | 0.00001 | SEQ ID No: 63 | + | + |
| gata binding protein 3 | | | | (nm_001002295) | | |
| EIF2C3 | ughs.530333:186 | − | 0.00001 | SEQ ID | + | + |
| eukaryotic translation | | | | No: 212 | | |
| initiation factor 2c, 3 | | | | (nm_024852) | | |
| C4A | ughs.534847:186 | − | 0.00001 | SEQ ID | + | + |
| complement | | | | No: 635 | | |
| component 4b, | | | | (nm_001002029) | | |
| telomeric | | | | | | |
| TFF3 | ughs.82961:186 | − | 0.00001 | SEQ ID | + | + |
| trefoil factor 3 | | | | No: 535 | | |
| (intestinal) | | | | (nm_003226) | | |
| | N_A | − | 0.00003 | SEQ ID | + | + |
| | | | | No: 1125 | | |
| NAT1 | ughs.155956:186 | − | 0.00003 | SEQ ID | + | − |
| n-acetyltransferase 1 | | | | No: 109 | | |
| (arylamine n- | | | | (nm_000662) | | |
| acetyltransferase) | | | | | | |
| COL4A2 | ughs.508716:186 | − | 0.00003 | SEQ ID | + | − |
| collagen, type iv, alpha 2 | | | | No: 342 | | |
| | | | | (nm_001846) | | |
| RABEP1 | ughs.551518:186 | − | 0.00003 | SEQ ID | + | − |
| rabaptin, rab gtpase | | | | No: 927 | | |
| binding effector protein 1 | | | | (nm_004703) | | |
| | N_A | − | 0.00005 | SEQ ID | + | + |
| | | | | No: 1124 | | |
| RHOBTB3 | ughs.445030:186 | − | 0.00006 | SEQ ID | − | − |
| rho-related btb domain | | | | No: 124 | | |
| containing 3 | | | | (nm_014899) | | |
| CASKIN1/flj12650 | ughs.530863:186; | − | 0.00006 | SEQ ID | + | − |
| cask interacting protein 1 | ughs.470259:186 | | | No: 280 | | |
| | | | | (nm_020764) | | |
| | | | | or SEQ ID | | |
| | | | | No: 1110 | | |
| | | | | (NM_024522) | | |
| CXXC5 | ughs.189119:186 | − | 0.00009 | SEQ ID | + | + |
| cxxc finger 5 | | | | No: 297 | | |
| | | | | (nm_016463) | | |
| MAPT | ughs.101174:186 | − | 0.0001 | SEQ ID | + | + |
| microtubule-associated | | | | No: 791 | | |
| protein tau | | | | (nm_016835) | | |
| MGC24047 | ughs.29190:186 | − | 0.0001 | SEQ ID | + | − |
| chromosome 1 open | | | | No: 210 | | |
| reading frame 64 | | | | (nm_178840) | | |
| MGC45441 | ughs.488337:186 | − | 0.00026 | SEQ ID | + | + |
| hypothetical protein | | | | No: 827 | | |
| mgc45441 | | | | (nm_152499) | | |
| CYP2B6 | N_A | − | 0.00065 | SEQ ID | − | − |
| Cytochrome P450, | | | | No: 1064 | | |
| family 2, subfamily B, | | | | (NM_000767) | | |
| polypeptide 6 | | | | | | |
| CROCC | ughs.309403:186; | − | 0.00072 | SEQ ID | − | − |
| ciliary rootlet coiled- | ughs.135718:186 | | | No: 147 | | |
| coil, rootletin | | | | (nm_014675) | | |
| USP21 | ughs.8015:186 | − | 0.00075 | SEQ ID | − | − |
| ubiquitin specific | | | | No: 323 | | |
| protease 21 | | | | (nm_001014443) | | |
| TRAF5 | ughs.523930:186 | − | 0.0011 | SEQ ID | − | − |
| tnf receptor-associated | | | | No: 106 | | |
| factor 5 | | | | (nm_004619) | | |
| GSTM2 | ughs.279837:186 | − | 0.00127 | SEQ ID | + | − |
| glutathione s- | | | | No: 181 | | |
| transferase m2 | | | | (nm_000848) | | |
| (muscle) | | | | | | |
| DUSP4 | ughs.417962:186 | − | 0.0015 | SEQ ID | − | − |
| dual specificity | | | | No: 376 | | |
| phosphatase 4 | | | | (nm_057158) | | |
| ASF1A | ughs.292316:186 | − | 0.00177 | SEQ ID | + | − |
| asf1 anti-silencing | | | | No: 116 | | |
| function 1 homolog a | | | | (nm_014034) | | |
| (s. cerevisiae) | | | | | | |
| CSF2 | ughs.1349:186 | − | 0.0024 | SEQ ID | − | − |
| colony stimulating | | | | No: 252 | | |
| factor 2 (granulocyte- | | | | (nm_000758) | | |
| macrophage) | | | | | | |
| CLSTN2 | ughs.158529:186 | − | 0.00247 | SEQ ID | − | − |
| calsyntenin 2 | | | | No: 797 | | |
| | | | | (nm_022131) | | |
| GLI3 | ughs.199338:186 | − | 0.00282 | SEQ ID | − | − |
| gli-kruppel family | | | | No: 911 | | |
| member gli3 (greig | | | | (nm_000168) | | |
| cephalopolysyndactyly | | | | | | |
| syndrome) | | | | | | |
| REPS2 | ughs.186810:186; | − | 0.00307 | SEQ ID | − | − |
| ralbp1 associated eps | ughs.131188:186 | | | No: 720 | | |
| domain containing 2 | | | | (nm_004726) | | |
| GSTM1 | ughs.301961:186 | − | 0.00307 | SEQ ID | − | − |
| glutathione s- | | | | No: 889 | | |
| transferase m1 | | | | (nm_000561) | | |
| PLAT | ughs.491582:186 | − | 0.00335 | SEQ ID | + | − |
| plasminogen activator, | | | | No: 250 | | |
| tissue | | | | (nm_000930) | | |
| DLG5 | ughs.500245:186 | − | 0.00393 | SEQ ID | − | − |
| discs, large homolog 5 | | | | No: 179 | | |
| (drosophila) | | | | (nm_004747) | | |
| FLJ00012 | ughs.21051:186 | − | 0.00396 | SEQ ID | − | − |
| flj00012 protein | | | | No: 786 | | |
| | | | | (nm_033388) | | |
| SIDT2 | ughs.410977:186 | − | 0.00409 | SEQ ID | + | − |
| sid1 transmembrane | | | | No: 177 | | |
| family, member 2 | | | | (nm_015996) | | |
| | N_A | − | 0.00434 | SEQ ID | − | − |
| | | | | No: 1047 | | |
| | | | | (BC012900) | | |
| BCL9 | ughs.415209:186 | − | 0.00434 | SEQ ID | − | − |
| b-cell cll/lymphoma 9 | | | | No: 301 | | |
| | | | | (nm_004326) | | |
| USP13 | ughs.175322:186 | − | 0.00516 | SEQ ID | + | + |
| ubiquitin specific | | | | No: 207 | | |
| protease 13 | | | | (nm_003940) | | |
| (isopeptidase t-3) | | | | | | |
| DNALI1 | ughs.406050:186 | − | 0.00606 | SEQ ID | − | − |
| dynein, axonemal, light | | | | No: 936 | | |
| intermediate | | | | (nm_003462) | | |
| polypeptide 1 | | | | | | |
| FOXC1/RHOB | ughs.348883:186; | − | 0.00652 | SEQ ID | + | + |
| forkhead box c1/ras | ughs.502876:186 | | | No: 916 | | |
| homolog gene | | | | (nm_001453) | | |
| | | | | or SEQ ID | | |
| | | | | No: 1116 | | |
| | | | | (NM_004040) | | |
| | N_A | − | 0.00699 | SEQ ID | + | + |
| | | | | No: 1052 | | |
| | | | | (BX096026) | | |
| KRT18 | ughs.406013:186 | − | 0.00879 | SEQ ID | + | + |
| keratin 18 | | | | No: 159 | | |
| | | | | (nm_000224) | | |
| | ughs.548040:186 | − | 0.00889 | SEQ ID | − | − |
| | | | | No: 1096 | | |
| | | | | (AK127274) | | |
| DNAJC12 | ughs.260720:186 | − | 0.0094 | SEQ ID No: 28 | − | − |
| dnaj (hsp40) homolog, | | | | (nm_021800) | | |
| subfamily c, member | | | | | | |
| 12 | | | | | | |
| cdna flj41270 fis, clone | ughs.445414:186 | − | 0.00963 | SEQ ID | − | − |
| bramy2036387 | | | | No: 1054 | | |
| | | | | (AK123264) | | |
| SPDEF/c8orf13 | ughs.124299:186; | − | 0.00981 | SEQ ID No: 25 | + | + |
| sam pointed domain | ughs.485158:186 | | | (nm_012391) | | |
| containing ets | | | | or SEQ ID | | |
| transcription factor/ | | | | No: 1108 | | |
| chromosome 8 open | | | | (NM_053279) | | |
| reading frame 13 | | | | | | |
| C20ORF23 | ughs.101774:186 | − | 0.01019 | SEQ ID | + | − |
| chromosome 20 open | | | | No: 825 | | |
| reading frame 23 | | | | (nm_024704) | | |
| FLJ20366 | ughs.390738:186 | − | 0.01278 | SEQ ID | + | − |
| hypothetical protein | | | | No: 145 | | |
| flj20366 | | | | (nm_017786) | | |
| COX6C | ughs.351875:186 | − | 0.01401 | SEQ ID | − | − |
| cytochrome c oxidase | | | | No: 491 | | |
| subunit vic | | | | (nm_004374) | | |
| RGS11 | ughs.65756:186 | − | 0.01422 | SEQ ID | − | − |
| regulator of g-protein | | | | No: 485 | | |
| signalling 11 | | | | (nm_003834) | | |
| Hypothetical protein | ughs.508559:186 | − | 0.01475 | SEQ ID | − | − |
| LOC153561 | | | | No: 1072 | | |
| | | | | (AY007114) | | |
| SEMA6B | ughs.465642:186 | − | 0.01572 | SEQ ID | − | − |
| sema domain, | | | | No: 274 | | |
| transmembrane | | | | (nm_032108) | | |
| domain (tm), and | | | | | | |
| cytoplasmic domain, | | | | | | |
| (semaphorin) 6b | | | | | | |
| AP1G2 | ughs.343244:186 | − | 0.01707 | SEQ ID | − | − |
| adaptor-related protein | | | | No: 258 | | |
| complex 1, gamma 2 | | | | (nm_080545) | | |
| subunit | | | | | | |
| AKAP8L | ughs.399800:186 | − | 0.01817 | SEQ ID | − | − |
| a kinase (prka) anchor | | | | No: 292 | | |
| protein 8-like | | | | (nm_014371) | | |
| PRKCBP1 | ughs.446240:186 | − | 0.01835 | SEQ ID | − | − |
| protein kinase c | | | | No: 803 | | |
| binding protein 1 | | | | (nm_183047) | | |
| CENTG3 | ughs.195048:186 | − | 0.02053 | SEQ ID | − | − |
| centaurin, gamma 3 | | | | No: 349 | | |
| | | | | (nm_031946) | | |
| genomic region on | ughs.159853:186 | − | 0.02456 | SEQ ID | − | − |
| chromosome 1 | | | | No: 1123 | | |
| SLC40A1 | ughs.529285:186 | − | 0.02463 | SEQ ID | − | − |
| solute carrier family 40 | | | | No: 763 | | |
| (iron-regulated | | | | (nm_014585) | | |
| transporter), member 1 | | | | | | |
| CCND2 | ughs.376071:186 | − | 0.02723 | SEQ ID | − | − |
| cyclin d2 | | | | No: 438 | | |
| | | | | (nm_001759) | | |
| KLHDC2 | N_A | − | 0.02795 | SEQ ID No: 94 | − | − |
| kelch domain | | | | (nm_014315) | | |
| containing 2 | | | | | | |
| ABCA3 | ughs.26630:186 | − | 0.03438 | SEQ ID | + | + |
| atp-binding cassette, | | | | No: 845 | | |
| sub-family a (abc1), | | | | (nm_001089) | | |
| member 3 | | | | | | |
| LOC143381 | ughs.388347:186; | − | 0.03705 | SEQ ID | − | − |
| hypothetical protein | ughs.557061:186 | | | No: 1084 | | |
| loc143381 | | | | (BX648964) | | |
| FLJ21439 | ughs.550536:186 | − | 0.03746 | SEQ ID | − | − |
| hypothetical protein | | | | No: 734 | | |
| flj21439 | | | | (nm_025137) | | |
| HOXA4 | ughs.77637:186 | − | 0.03897 | SEQ ID | − | − |
| homeo box a4 | | | | No: 943 | | |
| | | | | (nm_002141) | | |
| CACNA1D/KIF5C | ughs.476358:186; | − | 0.03958 | SEQ ID | + | + |
| Calcium channel, | ughs.435557:186 | | | No: 1085 | | |
| voltage-dependent, L | | | | (NM_000720) | | |
| type, alpha 1D | | | | | | |
| subunit/kinesin family | | | | | | |
| member 5c | | | | | | |
| GNG3 | ughs.179915:186 | − | 0.04937 | SEQ ID | + | − |
| guanine nucleotide | | | | No: 276 | | |
| binding protein (g | | | | (nm_012202) | | |
| protein), gamma 3 | | | | | | |

Multivariate Cox analysis allowed estimation of parameters corresponding to each of the selected metagenes:

| | Parameter | P value | |
|---|---|---|---|
| Metagene | estimation | (Chi square) | Hazard Ratio |
| UnderER | −2.90279 | 0.0906 | 0.055 |
| UnderPR | −1.47423 | 0.0143 | 0.229 |
| UnderEGFR | −4.17198 | 0.0012 | 0.015 |

On the basis of these parameters, the score for prognosis has been established as follows:

Score=−2.90279*underER−1.47423*underPR−4.17198*underEGFR

Threshold optimization: we tested all the possible thresholds. As an example 1^{st}, 2^{nd} and 3^{rd} quartile of the score distribution of the training set and found 0.502, 0.0057 and <0.0001 respectively for the p value associated to the log rank test.

The 3^{rd} quartile (cut-off=0.087646) was then defined as the optimal cut-off to separate patients into two groups with the highest significance.

The error on the score was integrated by calculating a confidence interval around the threshold, within which sample classification was considered non robust. Considering the score distribution Gaussian, we estimated the confidence interval around the threshold using standard deviation calculation method (estimated standard deviation of the population/√n).

The inventors have established that a woman having a score (S_{C}) of more than 0.136 have at least a double propensity of poor clinical outcome than a woman with a score (S_{C}) of less than 0.0393.

Model validation: the score was calculated for each of the 164 patients from the validation set and we separated the patients into two groups according the cut-off determined on the identification set. On the 164 patients, the model was well validated (p=4.7 10⁻⁰², log rank test) and separated the patients into a good-prognosis group with 80% 5-year MFS (84% of patients) and a poor-prognosis group with 63% 5-year MFS (13% of patients), 3% of patients being not interpretable. On a subset of the validation set, constituted of the clinical trial PACS01 (N=128), we obtained similar validation (p=3.9 10⁻⁰³, logrank test) with 88% of 5-year MFS in the good-prognosis group (80% of patients) and 65% of 5-year MFS in the poor-prognosis group (16% of patients, 4% of patients not interpretable).

Model performances: we performed multivariate analysis to determine the importance of the model as compared to standard clinical parameters. Even when considering grade, lymph node, ER status, age . . . , the model was still significant in the multivariate analysis, suggesting that it provides an independent, complementary and significant prognostic information.

Multivariate analysis on the global population (N=347)

| | | Hazard | CI95 | CI95 | |
|---|---|---|---|---|---|
| | | ratio | upper | lower | p |
| Age | <35 y | 1 | 0.16 | 2.79 | p = 0.57 |
| | >=35 y | 0.66 | | | |
| Menopausal | N | 1 | 0.82 | 1.9 | p = 0.31 |
| | Y | 1.24 | | | |
| Tumour size | pT1 | 1 | 0.95 | 2.74 | p = 0.078 |
| | pT2-pT3 | 1.61 | | | |
| N | N− | 1 | 0.76 | 2.76 | p = 0.26 |
| | N+ | 1.45 | | | |
| SBR grade | I | 1 | 0.96 | 5.35 | p = 0.062 |
| | II-III | 2.27 | | | |
| HR (10%) | HR− | 1 | 0.53 | 1.4 | p = 0.54 |
| | HR+ | 0.86 | | | |
| Erbb2 | 0-1-2 | 1 | 0.66 | 2.07 | p = 0.58 |
| | 3 | 1.17 | | | |
| Model | Good | 1 | 1.65 | 4.11 | P = 3.8 10⁻⁵ |
| | Poor | 2.61 | | | |

Multivariate analysis on the identification set (N=222)

| | | Hazard | CI95 | | |
|---|---|---|---|---|---|
| | | ratio | upper | CI95 lower | p |
| Age | <35 y | 1 | 0.19 | 10.8 | p = 0.73 |
| | >=35 y | 1.43 | | | |
| Menopausal | N | 1 | 0.64 | 1.68 | p = 0.89 |
| | Y | 1.03 | | | |
| Tumour size | pT1 | 1 | 0.63 | 1.97 | p = 0.7 |
| | pT2-pT3 | 1.12 | | | |
| N | N− | 1 | 0.9 | 3.34 | p = 0.1 |
| | N+ | 1.73 | | | |
| SBR grade | I | 1 | 0.87 | 5.08 | p = 0.098 |
| | II-III | 2.1 | | | |
| HR (10%) | HR− | 1 | 0.53 | 1.62 | p = 0.79 |
| | HR+ | 0.93 | | | |
| Erbb2 | 0-1-2 | 1 | 0.44 | 1.93 | p = 0.84 |
| | 3 | 0.93 | | | |
| Model | Good | 1 | 1.3 | 3.64 | P = 0.003 |
| | Poor | 2.18 | | | |

Multivariate analysis on the PACS01 clinical trial (N=108)

| | | | CI95 | CI95 | |
|---|---|---|---|---|---|
| | | Hazard ratio | upper | lower | p |
| Age | <35 y | 1 | 0 | Inf | p = 1 |
| | >=35 y | 518527625.4 | | | |
| Menopausal | N | 1 | 0.51 | 3.47 | p = 0.56 |
| | Y | 1.33 | | | |
| Tumour size | pT1 | 1 | 0 | Inf | p = 1 |
| | pT2-pT3 | 324628156.2 | | | |
| N | N− | 1 | | | p = NA |
| | N+ | | | | |
| SBR grade | I | 1 | 0 | Inf | p = 1 |
| | II-III | 287987535.5 | | | |
| HR (10%) | HR− | 1 | 0.36 | 5.61 | p = 0.62 |
| | HR+ | 1.42 | | | |
| Erbb2 | 0-1-2 | 1 | 0.68 | 6.66 | p = 0.19 |
| | 3 | 2.13 | | | |
| Model | Good | 1 | 1.58 | 17.74 | P = 0.0068 |
| | Poor | 5.3 | | | |

Metagenes Reduction:

In this model with underER, underPR and underEGFR, we defined the number of genes according to their significance in the metagene identification with the MaxT method. Even if the genes are well correlated between each other, some of them may be removed from further analysis, in order to reduce the number of genes to analyze and simplify the analysis process.

We calculated the correlation between each gene composing the metagene and the metagene, sorted the genes according to their increasing correlation to the metagene and progressively eliminated the genes the least correlated to the metagene, starting from 1 removed gene to all except one removed genes.

For each of these new sets of genes, we calculated a new metagene and its correlation with the original metagene. We selected given correlation cut-offs varying from 0.91 to 0.99 and integrated the corresponding new metagene in the model. This allowed us to generate a new score and prognostic group for each patient and to compare the attribution of a given prognostic group between the original model and the model with the optimized metagene. The criterion was equivalence between the 2 patients classification (with the original model and the optimized one) within the 2 prognostic groups.

As an example, we can reduce the number of genes from the metagene underER from 42 to 27 (Table I), while keeping 97% of equivalence (meaning that only 3% of patients are predicted in the opposite prognostic group when optimizing the metagene) for patient classification in the two prognostic groups on the validation set. With 20 genes (Table I), the concordancy is still of 95%.

In the same way, the metagene underPR may be reduced from 73 to 35 (Table II) and 6 genes (Table II) with 96% and 94% equivalence respectively for patient classification in the validation set.

The metagene underEGFR may be reduced from 71 to 34 (Table III) and 22 genes (Table III) with 95% and 91% concordancy respectively for patient classification in the validation set.

Considering optimization of the 3 metagenes, we reached on the validation set a concordancy of 91% and 90% with 102 and 50 genes respectively instead of the 186 genes used in the original model.

### Example 3

### Identification of a Second Significant Metagene Combination

Since ER and EGFR markers are correlated, with the majority of EGFR+ being ER−, we found another combination that could replace the metagenes underER and underPR by a single metagene overEGFR.

TABLE IV
(Metagene OverEGFR)

| | | | | | Reduced | Reduced |
|---|---|---|---|---|---|---|
| Gene | Unigene Cluster | Regulation | P value | Ref. Seq | Metagene 12 | Metagene 5 |
| GSTP1 | ughs.523836:186 | + | 0.00005 | SEQ ID | − | − |
| glutathione s- | | | | No: 405 | | |
| transferase pi | | | | (nm_000852) | | |
| ITGB3 | ughs.218040:186 | + | 0.00008 | SEQ ID | − | − |
| integrin, beta 3 | | | | No: 374 | | |
| (platelet | | | | (nm_000212) | | |
| glycoprotein iiia, | | | | | | |
| antigen cd61) | | | | | | |
| IGHG1 | ughs.510635:186 | + | 0.00011 | SEQ ID | + | + |
| immunoglobulin | | | | No: 1122 | | |
| heavy constant | | | | | | |
| gamma 1 (g1m | | | | | | |
| marker) | | | | | | |
| SOD2 | ughs.487046:186 | + | 0.00072 | SEQ ID | + | + |
| superoxide | | | | No: 598 | | |
| dismutase 2, | | | | (nm_000636) | | |
| mitochondrial | | | | | | |
| CEBPB | ughs.517106:186 | + | 0.00089 | SEQ ID | + | − |
| ccaat/enhancer | | | | No: 262 | | |
| binding protein | | | | (nm_005194) | | |
| (c/ebp), beta | | | | | | |
| IGKC | ughs.449621:186; | + | 0.00177 | SEQ ID | + | − |
| immunoglobulin | ughs.546620:186 | | | No: 1099 | | |
| kappa constant | | | | (BC066343) | | |
| ENO1 | ughs.517145:186 | + | 0.00201 | SEQ ID | + | + |
| enolase 1, | | | | No: 696 | | |
| (alpha) | | | | (nm_001428) | | |
| npc-a-5 | ughs.510543:186 | + | 0.00352 | SEQ ID | + | + |
| nasopharyngeal | | | | No: 1059 | | |
| carcinoma- | | | | (AK091113) | | |
| associated | | | | | | |
| antigen npc-a-5 | | | | | | |
| MMP7 | ughs.2256:186 | + | 0.00698 | SEQ ID | + | − |
| matrix | | | | No: 751 | | |
| metalloproteinase | | | | (nm_002423) | | |
| 7 (matrilysin, | | | | | | |
| uterine) | | | | | | |
| | N_A | + | 0.01196 | SEQ ID | + | − |
| | | | | No: 1121 | | |
| MKI67 | ughs.80976:186 | + | 0.0122 | SEQ ID | + | − |
| antigen identified | | | | No: 286 | | |
| by monoclonal | | | | (nm_002417) | | |
| antibody ki-67 | | | | | | |
| ARHGEF1 | ughs.278186:186 | + | 0.01427 | SEQ ID | − | − |
| rho guanine | | | | No: 244 | | |
| nucleotide | | | | (nm_199002) | | |
| exchange factor | | | | | | |
| (gef) 1 | | | | | | |
| ATF2 | ughs.425104:186 | + | 0.0148 | SEQ ID | − | − |
| activating | | | | No: 18 | | |
| transcription | | | | (nm_001880) | | |
| factor 2 | | | | | | |
| TFCP2L1 | ughs.156471:186 | + | 0.0259 | SEQ ID | + | + |
| transcription | | | | No: 121 | | |
| factor cp2-like 1 | | | | (nm_014553) | | |
| IGKC | N_A | + | 0.02767 | SEQ ID | − | − |
| Immunoglobulin | | | | No: 1107 | | |
| kappa variable 1-5 | | | | (BC073775) | | |
| (IGKC) | | | | | | |
| PRSS12 | ughs.445857:186 | + | 0.03118 | SEQ ID | + | − |
| protease, serine, | | | | No: 103 | | |
| 12 (neurotrypsin, | | | | (nm_003619) | | |
| motopsin) | | | | | | |
| IGLC2 | ughs.449585:186 | + | 0.04077 | SEQ ID | + | − |
| immunoglobulin | | | | No: 1118 | | |
| lambda joining 3 | | | | | | |
| CSF1 | ughs.173894:186 | + | 0.0412 | SEQ ID | − | − |
| colony | | | | No: 42 | | |
| stimulating factor | | | | (nm_000757) | | |
| 1 (macrophage) | | | | | | |
| LOC114659 | ughs.406166:186; | + | 0.04453 | SEQ ID | − | − |
| SH3-domain | ughs.438861:186 | | | No: 1067 | | |
| GRB2-like | | | | (AK123784) | | |
| pseudogene 1 | | | | | | |

Multivariate Cox analysis allowed estimation of parameters corresponding to each of the selected metagenes:

| | Parameter | P value | |
|---|---|---|---|
| Metagene | estimation | (Chi square) | Hazard Ratio |
| OverEGFR | −1.33 | 0.022 | 0.26 |
| UnderEGFR | −2.28 | 0.0048 | 0.10 |

On the basis of these parameters, the score for prognosis has been established as follows:

Score=−1.33*overEGFR−2.28*underEGFR

Threshold optimization: the 3^{rd} quartile was selected (cut-off=0.14) associated with a [0.103-0.177] confidence interval, separating patients into two groups of 79% 5years MFS in the good prognosis group and 60% of 5 years MFS in the poor prognosis group (p=0.041, logrank test).

Model validation: we calculated the score for the 164 patients of the validation set with the formula identified on the training set, and separated the patients according to the defined threshold. The model was well validated (p=1.1 10⁻⁰³, log rank test), with 82% MFS at 5 years in the good prognosis group (76% of patients), and 54% MFS in the poor prognosis group (20% of patients, 5% of patients not interpretable). On a subset of the validation set, constituted of the clinical trial PACS01 (N=128), we obtained similar validation (p=2.9 10⁻⁰³, logrank test) with 87% of 5-year MFS in the good-prognosis group (75% of patients) and 60% of 5-year MFS in the poor-prognosis group (19% of patients, 6% of patients not interpretable).

Model performances: we performed multivariate analysis to determine the importance of the model as previously.

Multivariate analysis on the global population (N=347)

| | | Hazard | CI95 | | |
|---|---|---|---|---|---|
| | | ratio | upper | CI95 lower | p |
| Age | <35 y | 1 | 0.17 | 3.09 | p = 0.67 |
| | >=35 y | 0.73 | | | |
| Menopausal | N | 1 | 0.83 | 1.93 | p = 0.27 |
| | Y | 1.27 | | | |
| Tumour size | pT1 | 1 | 0.97 | 2.79 | p = 0.065 |
| | pT2-pT3 | 1.65 | | | |
| N | N− | 1 | 0.73 | 2.64 | p = 0.32 |
| | N+ | 1.39 | | | |
| SBR grade | I | 1 | 1.05 | 5.78 | p = 0.039 |
| | II-III | 2.46 | | | |
| HR (10%) | HR− | 1 | 0.48 | 1.33 | p = 0.4 |
| | HR+ | 0.8 | | | |
| Erbb2 | 0-1-2 | 1 | 0.59 | 1.85 | p = 0.88 |
| | 3 | 1.05 | | | |
| Model | Good | 1 | 1.09 | 2.94 | P = 0.021 |
| | Poor | 1.79 | | | |

Multivariate analysis on the training set (N=222)

| | | Hazard | CI95 | | |
|---|---|---|---|---|---|
| | | ratio | upper | CI95 lower | p |
| Age | <35 y | 1 | 0.22 | 12.59 | p = 0.62 |
| | >=35 y | 1.67 | | | |
| Menopausal | N | 1 | 0.63 | 1.65 | p = 0.95 |
| | Y | 1.01 | | | |
| Tumour size | pT1 | 1 | 0.63 | 1.97 | p = 0.71 |
| | pT2-pT3 | 1.11 | | | |
| N | N− | 1 | 0.86 | 3.21 | p = 0.13 |
| | N+ | 1.67 | | | |
| SBR grade | I | 1 | 0.95 | 5.46 | p = 0.067 |
| | II-III | 2.27 | | | |
| HR (10%) | HR− | 1 | 0.48 | 1.57 | p = 0.65 |
| | HR+ | 0.87 | | | |
| Erbb2 | 0-1-2 | 1 | 0.4 | 1.77 | p = 0.66 |
| | 3 | 0.85 | | | |
| Model | Good | 1 | 0.73 | 2.38 | P = 0.35 |
| | Poor | 1.32 | | | |

Multivariate analysis on the PACS01 clinical trial (N=108)

| | | | CI95 | CI95 | |
|---|---|---|---|---|---|
| | | Hazard ratio | upper | lower | p |
| Age | <35 y | 1 | 0 | Inf | p = 1 |
| | >=35 y | 440091063.3 | | | |
| Menopausal | N | 1 | 0.62 | 4.12 | p = 0.34 |
| | Y | 1.59 | | | |
| Tumour size | pT1 | 1 | 0 | Inf | p = 1 |
| | pT2-pT3 | 267234385.6 | | | |
| N | N− | 1 | | | p = NA |
| | N+ | | | | |
| SBR grade | I | 1 | 0 | Inf | p = 1 |
| | II-III | 182875754.1 | | | |
| HR (10%) | HR− | 1 | 0.26 | 3.5 | p = 0.94 |
| | HR+ | 0.95 | | | |
| Erbb2 | 0-1-2 | 1 | 0.65 | 6.1 | p = 0.23 |
| | 3 | 1.99 | | | |
| Model | Good | 1 | 0.96 | 10.02 | P = 0.059 |
| | Poor | 3.09 | | | |

Metagenes Reduction:

We optimized the number of genes to analyse in underEGFR and overEGFR signature as described previously for the other metagenes.

The metagene overEGFR could be reduced from 19 to 12 (Table IV) or 5 genes (Table IV) with a concordancy of 96% and 94% respectively on the validation set.

Taken with the optimized underEGFR metagene, we obtained a concordancy of 95 and 91% considering 37 (Table III) and 24 genes (Table III) respectively instead of 92.

Some metagenes could be reduced at the level of a single gene still having a significant prognostic value.

An example of such a gene-based model contains SCUBE2 (SEQ ID NO: 681) and IGKC (SEQ ID NO: 1107 or 1099). SCUBE2 is an element of underEGFR metagene, while IGKC is part of overEGFR metagene.

| | Parameter | P value | |
|---|---|---|---|
| Metagene | estimation | (Chi square) | Hazard Ratio |
| SCUBE2 | −0.746 | 0.0016 | 0.474 |
| IGKC | −0.463 | 0.037 | 0.629 |

Threshold optimization: the 3^{rd} quartile (cut-off=0.095), confidence interval [0.0513-0.1387]) was the most significant (p=9.1 10⁻⁰⁴, logrank test) and separated the identification set in a good-prognosis group (77% MFS at 5 years) and a poor-prognosis group (51% MFS at 5 years).

Model Validation: we used the coefficients and the threshold previously calculated to separate the 164 patients from the validation set into two groups that had statistically significant outcome (p=4 10⁻⁰⁴, logrank test). The good prognosis group had a 5 y MFS of 83% (69% of the patients) while the poor prognosis group had a 5 y MFS of 55% (24% of the patients, 7% of patients not interpretable). On a subset of the validation set, constituted of the clinical trial PACS01 (N=128), we obtained similar validation (p=1.3 10⁻⁰³, logrank test) with 90% of 5-year MFS in the good-prognosis group (69% of patients) and 61% of 5-year MFS in the poor-prognosis group (23% of patients, 7% of patients not interpretable).

Model performances: we performed multivariate analysis to determine the importance of this simplified model as described previously.

Multivariate analysis on the global population (N=330)

| | | Hazard | CI95 | | |
|---|---|---|---|---|---|
| | | ratio | upper | CI95 lower | p |
| Age | <35 y | 1 | 0.22 | 3.77 | p = 0.89 |
| | >=35 y | 0.91 | | | |
| Menopausal | N | 1 | 0.78 | 1.85 | p = 0.4 |
| | Y | 1.2 | | | |
| Tumour size | pT1 | 1 | 0.95 | 2.74 | p = 0.079 |
| | pT2-pT3 | 1.61 | | | |
| N | N− | 1 | 0.72 | 2.64 | p = 0.33 |
| | N+ | 1.38 | | | |
| SBR grade | I | 1 | 1 | 5.56 | p = 0.051 |
| | II-III | 2.36 | | | |
| HR (10%) | HR− | 1 | 0.44 | 1.13 | p = 0.15 |
| | HR+ | 0.71 | | | |
| Erbb2 | 0-1-2 | 1 | 0.62 | 1.92 | p = 0.76 |
| | 3 | 1.09 | | | |
| Model | Good | 1 | 1.17 | 2.82 | P = 0.0077 |
| | Poor | 1.82 | | | |

Multivariate analysis on the training set (N=222)

| | | Hazard | CI95 | | |
|---|---|---|---|---|---|
| | | ratio | upper | CI95 lower | p |
| Age | <35 y | 1 | 0.21 | 11.94 | p = 0.65 |
| | >=35 y | 1.59 | | | |
| Menopausal | N | 1 | 0.61 | 1.6 | p = 0.97 |
| | Y | 0.99 | | | |
| Tumour size | pT1 | 1 | 0.62 | 1.94 | p = 0.75 |
| | pT2-pT3 | 1.1 | | | |
| N | N− | 1 | 0.85 | 3.17 | p = 0.14 |
| | N+ | 1.64 | | | |
| SBR grade | I | 1 | 0.87 | 5.12 | p = 0.098 |
| | II-III | 2.11 | | | |
| HR (10%) | HR− | 1 | 0.52 | 1.59 | p = 0.74 |
| | HR+ | 0.91 | | | |
| Erbb2 | 0-1-2 | 1 | 0.44 | 1.89 | p = 0.8 |
| | 3 | 0.91 | | | |
| Model | Good | 1 | 1.02 | 2.99 | P = 0.043 |
| | Poor | 1.74 | | | |

Multivariate analysis on the PACS01 clinical trial (N=108)

| | | | |
|---|---|---|---|
| | CI95 | CI95 | |
| Hazard ratio | upper | lower | p |

| | | | | | |
|---|---|---|---|---|---|
| Age | <35 y | 1 | 0.09 | 6.22 | p = 0.77 |
| | >=35 y | 0.73 | | | |
| Menopausal | N | 1 | 0.37 | 2.73 | p = 0.99 |
| | Y | 1.01 | | | |
| Tumour size | pT1 | 1 | 0.79 | 48.7 | p = 0.083 |
| | pT2-pT3 | 6.19 | | | |
| N | N− | 1 | | | p = NA |
| | N+ | | | | |
| SBR grade | I | 1 | 0 | Inf | p = 1 |
| | II-III | 634794463.76 | | | |
| HR (10%) | HR− | 1 | 0.23 | 1.58 | p = 0.3 |
| | HR+ | 0.6 | | | |
| Erbb2 | 0-1-2 | 1 | 0.8 | 6.74 | p = 0.12 |
| | 3 | 2.32 | | | |
| Model | Good | 1 | 1.01 | 6.06 | P = 0.049 |
| | Poor | 2.47 | | | |

Different nucleic acids array platforms may be used to work the present invention including, but not limited to, cDNA platforms (Image or “Ipso” clones described below), Affymetrix® platforms (GeneChip® probe sets) and others.

### Example 4

### Use of Metagenes Combinations According to the Invention on a cDNA Platform

The following tables are examples of metagenes of the invention that may be used on a cDNA platform according to the above described methods. For example, the following underER, underPR and underEGFR metagenes may be used in the above described method using a Cox regression analysis and the score S_{C}=−2.90279×underER−1.47423×underPR−4.17198×under EGFR, with the intervals mentioned previously in the description for “a”, “b” and “c” (and similarly for the above described combination involving underEGFR and over EGFR, as well as the IGKC+SCUBE2 combination). The Seq3′ and Seq5′ in the tables below columns provide the sequences identifying the respective Image or Ipso clones.

TABLE V
Metagene UnderER

| Set | Gene | | | | | | Seq | Seq | |
|---|---|---|---|---|---|---|---|---|---|
| No. | symbol | Clone ID | Gene name | Unigene Cluster | Regulation | P value | 3′ | 5′ | Ref. Seq |
| 402 | ITGB3 | ipso:0000143 | integrin, beta 3 | ughs.218040:186 | − | 0.00001 | | SEQ ID No: 992 | SEQ ID No: |
| | | | (platelet | | | | | | 374 |
| | | | glycoprotein iiia, | | | | | | (nm_000212) |
| | | | antigen cd61) | | | | | | |
| 423 | PADI2 | ipso:0000610 | peptidyl arginine | ughs.33455:186 | − | 0.00001 | | SEQ ID | SEQ ID No: 1027 |
| | | | deiminase, type ii | | | | | No: 1026 | (nm_007365) |
| 246 | SOD2 | image:324014 | superoxide | ughs.487046:186 | − | 0.00001 | SEQ ID | | SEQ ID No: 598 |
| | | | dismutase 2, | | | | No: 597 | | (nm_000636) |
| | | | mitochondrial | | | | | | |
| 290 | FLJ13154 | image:43457 | hypothetical protein | ughs.408702:186 | − | 0.00003 | SEQ ID | SEQ ID No: 716 | SEQ ID No: 717 |
| | | | flj13154 | | | | No: 715 | | (nm_024598) |
| 237 | HDAC2 | image:309924 | histone deacetylase 2 | ughs.3352:186 | − | 0.00004 | SEQ ID | SEQ ID No: 572 | SEQ ID No: 573 |
| | | | | | | | No: 571 | | (nm_001527) |
| 34 | SLAC2-B | image:142546 | slac2-b | N_A | − | 0.00006 | | SEQ ID No: 82 | SEQ ID No: 83 |
| | | | | | | | | | (nm_015065) |
| 6 | S100A8 | image:1089513 | s100 calcium | ughs.416073:186 | − | 0.00006 | SEQ ID | | SEQ ID No: 12 |
| | | | binding protein a8 | | | | No: 11 | | (nm_002964) |
| | | | (calgranulin a) | | | | | | |
| 171 | GSTP1 | image:231424 | glutathione s- | ughs.523836:186 | − | 0.00006 | SEQ ID | SEQ ID No: 404 | SEQ ID No: 405 |
| | | | transferase pi | | | | No: 403 | | (nm_000852) |
| 343 | LCN2 | image:544683 | lipocalin 2 | ughs.204238:186 | − | 0.00012 | SEQ ID | SEQ ID No: 855 | SEQ ID No: 856 |
| | | | (oncogene 24p3) | | | | No: 854 | | (nm_005564) |
| 163 | MYBL2 | image:207378 | v-myb | ughs.179718:186 | − | 0.00013 | SEQ ID | SEQ ID No: 383 | SEQ ID No: 384 |
| | | | myeloblastosis viral | | | | No: 382 | | (nm_002466) |
| | | | oncogene homolog | | | | | | |
| | | | (avian)-like 2 | | | | | | |
| 69 | PFKP | image:152714 | phosphofructokinase, | ughs.26010:186 | − | 0.00081 | SEQ ID | SEQ ID No: 166 | SEQ ID No: 167 |
| | | | platelet | | | | No: 165 | | (nm_002627) |
| 152 | STK6 | image:1912132 | serine/threonine | ughs.250822:186 | − | 0.00134 | SEQ ID | | SEQ ID No: 51 |
| | | | kinase 6 | | | | No: 358 | | (nm_198433) |
| 408 | GPR125 | ipso:0000267 | g protein-coupled | ughs.99195:186 | − | 0.00153 | | SEQ ID | SEQ ID No: 999 |
| | | | receptor 125 | | | | | No: 1001 | (nm_145290) |
| 393 | DSCR1 | ipso:0000077 | down syndrome | ughs.282326:186 | − | 0.00206 | | SEQ ID No: 978 | SEQ ID No: 979 |
| | | | critical region gene 1 | | | | | | (nm_004414) |
| 1 | FAT | image:1028762 | fat tumor | ughs.481371:186 | − | 0.0023 | SEQ ID | | SEQ ID No: 2 |
| | | | suppressor homolog | | | | No: 1 | | (nm_005245) |
| | | | 1 (drosophila) | | | | | | |
| 40 | VGLL1 | image:143622 | vestigial like 1 | N_A | − | 0.00247 | SEQ ID | SEQ ID No: 97 | SEQ ID No: 98 |
| | | | (drosophila) | | | | No: 96 | | (nm_016267) |
| 302 | MMP7 | image:471134 | matrix | ughs.2256:186 | − | 0.00264 | SEQ ID | SEQ ID No: 750 | SEQ ID No: 751 |
| | | | metalloproteinase 7 | | | | No: 749 | | (nm_002423) |
| | | | (matrilysin, uterine) | | | | | | |
| 282 | ENO1 | image:392678 | enolase 1, (alpha) | ughs.517145:186 | − | 0.00348 | SEQ ID | | SEQ ID No: 696 |
| | | | | | | | No: 695 | | (nm_001428) |
| 59 | | image:1493187 | cdna clone | ughs.175285:186 | − | 0.00429 | SEQ ID | SEQ ID | SEQ ID No: 1050 |
| | | | image:4831215 | | | | No: 142 | No: 1051 | (BC034638) |
| 203 | SCP2 | image:278490 | sterol carrier protein 2 | ughs.476365:186 | − | 0.00469 | SEQ ID | SEQ ID No: 487 | SEQ ID No: 488 |
| | | | | | | | No: 486 | | (nm_002979) |
| 111 | CEBPB | image:161993 | ccaat/enhancer | ughs.517106:186 | − | 0.00507 | SEQ ID | | SEQ ID No: 262 |
| | | | binding protein | | | | No: 261 | | (nm_005194) |
| | | | (c/ebp), beta | | | | | | |
| 419 | TGM1 | ipso:0000488 | transglutaminase 1 | ughs.508950:186 | − | 0.00695 | | SEQ ID | SEQ ID No: 1020 |
| | | | (k polypeptide | | | | | No: 1019 | (nm_000359) |
| | | | epidermal type i, | | | | | | |
| | | | protein-glutamine- | | | | | | |
| | | | gamma- | | | | | | |
| | | | glutamyltransferase) | | | | | | |
| 418 | | ipso:0000487 | | N_A | − | 0.00764 | | SEQ ID | SEQ ID No: 1106 |
| | | | | | | | | No: 1018 | (BC015969) |
| 380 | GGH | image:809588 | gamma-glutamyl | ughs.78619:186 | − | 0.00881 | SEQ ID | SEQ ID No: 951 | SEQ ID No: 952 |
| | | | hydrolase | | | | No: 950 | | (nm_003878) |
| | | | (conjugase, | | | | | | |
| | | | folylpolygamma- | | | | | | |
| | | | glutamyl hydrolase) | | | | | | |
| 273 | GSTA4 | image:345309 | glutathione s- | ughs.485557:186 | − | 0.00995 | SEQ ID | SEQ ID No: 674 | SEQ ID No: 675 |
| | | | transferase a4 | | | | No: 673 | | (nm_001512) |
| 123 | FN5 | image:171580 | b-cell cll/lymphoma | ughs.438064:186 | − | 0.0109 | SEQ ID | SEQ ID No: 288 | SEQ ID No: 289 |
| | | | 7b | | | | No: 287 | | (nm_020179) |
| 387 | CCNB2 | image:845594 | glutamate | ughs.194698:186 | − | 0.01221 | SEQ ID | | SEQ ID No: 553 |
| | | | decarboxylase 1 | | | | No: 969 | | (nm_004701) |
| | | | (gad 1) | | | | | | |
| 239 | CTSC | image:320656 | cathepsin c | ughs.128065:186 | − | 0.01501 | SEQ ID | SEQ ID No: 578 | SEQ ID No: 579 |
| | | | | | | | No: 577 | | (nm_001814) |
| 305 | PBEF1 | image:488548 | pre-b-cell colony | ughs.489615:186 | − | 0.01621 | SEQ ID | SEQ ID No: 759 | SEQ ID No: 760 |
| | | | enhancing factor 1 | | | | No: 758 | | (nm_005746) |
| 323 | S100A6 | image:512420 | s100 calcium | ughs.275243:186 | − | 0.01719 | SEQ ID | | SEQ ID No: 805 |
| | | | binding protein a6 | | | | No: 804 | | (nm_014624) |
| | | | (calcyclin) | | | | | | |
| 153 | RDX | image:193081 | radixin | ughs.263671:186 | − | 0.01753 | SEQ ID | SEQ ID No: 360 | SEQ ID No: 361 |
| | | | | | | | No: 359 | | (nm_002906) |
| 187 | GPR126 | image:259884 | g protein-coupled | ughs.318894:186 | − | 0.01886 | SEQ ID | SEQ ID No: 447 | SEQ ID No: 448 |
| | | | receptor 126 | | | | No: 446 | | (nm_198569) |
| 70 | MMP15 | image:152744 | matrix | ughs.80343:186 | − | 0.0274 | SEQ ID | SEQ ID No: 169 | SEQ ID No: 170 |
| | | | metalloproteinase | | | | No: 168 | | (nm_002428) |
| | | | 15 (membrane- | | | | | | |
| | | | inserted) | | | | | | |
| 352 | KLK6 | image:724109 | kallikrein 6 | ughs.79361:186 | − | 0.02892 | SEQ ID | SEQ ID No: 877 | SEQ ID No: 878 |
| | | | (neurosin, zyme) | | | | No: 876 | | (nm_002774) |
| 79 | | image: 153978 | | N_A | − | 0.0351 | SEQ ID | SEQ ID No: 190 | SEQ ID No: 1117 |
| | | | | | | | No: 189 | | |
| 251 | BOK | image:325789 | bcl2-related ovarian | ughs.293753:186 | − | 0.03747 | SEQ ID | | SEQ ID No: 612 |
| | | | killer | | | | No: 611 | | (nm_032515) |
| 225 | CDKL5 | image:301018 | cyclin-dependent | ughs.435570:186 | − | 0.03754 | SEQ ID | SEQ ID No: 539 | SEQ ID No: 540 |
| | | | kinase-like 5 | | | | No: 538 | | (nm_003159) |
| 330 | CSTB | image:51814 | cystatin b (stefin b) | ughs.695:186 | − | 0.0382 | SEQ ID | SEQ ID No: 822 | SEQ ID No: 823 |
| | | | | | | | No: 821 | | (nm_000100) |
| 54 | LOC151194 | image:147707 | similar to | ughs.552610:186 | − | 0.03884 | | SEQ ID No: 130 | SEQ ID No: 131 |
| | | | hepatocellular | | | | | | (nm_145280) |
| | | | carcinoma- | | | | | | |
| | | | associated antigen | | | | | | |
| | | | hca557b | | | | | | |
| 285 | NFIB | image:416959 | nuclear factor i/b | ughs.370359:186 | − | 0.03949 | SEQ ID | SEQ ID No: 704 | SEQ ID No: 705 |
| | | | | | | | No: 703 | | (nm_005596) |
| 14 | LAD1 | image:121551 | ladinin 1 | ughs.519035:186 | − | 0.04184 | SEQ ID | SEQ ID No: 30 | SEQ ID No: 31 |
| | | | | | | | No: 29 | | (nm_005558) |
| 83 | MGC11271 | image:154651 | hypothetical protein | ughs.143288:186 | − | 0.04312 | | SEQ ID No: 198 | SEQ ID No: 199 |
| | | | mgc11271 | | | | | | (nm_024323) |

TABLE VI
Metagene underPR

| Set | Gene | | | | | | | | |
|---|---|---|---|---|---|---|---|---|---|
| No. | symbol | Clone ID | Gene name | Unigene Cluster | Regulation | P value | Seq3′ | Seq5′ | Ref. Seq |
| 246 | SOD2 | image:324014 | superoxide | ughs.487046:186 | − | 1E−05 | SED ID | | SED ID No: 598 |
| | | | dismutase 2, | | | | No: 597 | | (nm_000636) |
| | | | mitochondrial | | | | | | |
| 217 | IGHG1 | image:289337 | immunoglobulin | ughs.510635:186 | − | 1E−05 | SED ID | SED ID No: 521 | SED ID No: 1122 |
| | | | heavy constant | | | | No: 520 | | |
| | | | gamma 1 (g1m | | | | | | |
| | | | marker) | | | | | | |
| 154 | KDR | image:193857 | kinase insert domain | ughs.479756:186 | − | 0.0001 | SED ID | SED ID No: 363 | SED ID No: 364 |
| | | | receptor (a type iii | | | | No: 362 | | (nm_002253) |
| | | | receptor tyrosine | | | | | | |
| | | | kinase) | | | | | | |
| 164 | KLF1 | image:208991 | kruppel-like factor 1 | ughs.37860:186 | − | 0.0001 | SEQ ID | SEQ ID No: 386 | SEQ ID No: 387 |
| | | | (erythroid) | | | | No: 385 | | (nm_006563) |
| 15 | CASP9 | image:121693 | caspase 9, | ughs.329502:186 | − | 0.0002 | SEQ ID | SEQ ID No: 33 | SEQ ID No: 34 |
| | | | apoptosis-related | | | | No: 32 | | (nm_001229) |
| | | | cysteine protease | | | | | | |
| 267 | BCL2 | image:342181 | b-cell cll/lymphoma 2 | ughs.150749:186 | − | 0.0002 | SEQ ID | SEQ ID No: 656 | SEQ ID No: 657 |
| | | | | | | | No: 655 | | (nm_000633) |
| 163 | MYBL2 | image:207378 | v-myb | ughs.179718:186 | − | 0.0003 | SEQ ID | SEQ ID No: 383 | SEQ ID No: 384 |
| | | | myeloblastosis viral | | | | No: 382 | | (nm_002466) |
| | | | oncogene homolog | | | | | | |
| | | | (avian)-like 2 | | | | | | |
| 188 | ADAM10 | image:261401 | a disintegrin and | ughs.172028:186 | − | 0.0003 | SEQ ID | SEQ ID No: 450 | SEQ ID No: 451 |
| | | | metalloproteinase | | | | No: 449 | | (nm_001110) |
| | | | domain 10 | | | | | | |
| 406 | GPR125 | ipso:0000252 | g protein-coupled | ughs.99195:186 | − | 0.0003 | | SEQ ID No: 998 | SEQ ID No: 999 |
| | | | receptor 125 | | | | | | (nm_145290) |
| 81 | | image:154483 | | ughs.26192:186 | − | 0.0005 | SEQ ID | SEQ ID | SEQ ID No: 1056 |
| | | | | | | | No: 194 | No: 1055 | (AK126297) |
| 7 | TGFBR3 | image:110287 | transforming growth | ughs.482390:186 | − | 0.0006 | SEQ ID | SEQ ID No: 14 | SEQ ID No: 15 |
| | | | factor, beta receptor | | | | No: 13 | | (nm_003243) |
| | | | iii (betaglycan, | | | | | | |
| | | | 300 kda) | | | | | | |
| 318 | LOC91316 | image:50877 | similar to bk246h3.1 | ughs.407693:186; | − | 0.0007 | SEQ ID | SEQ ID | SEQ ID No: 1090 |
| | | | (immunoglobulin | ughs.148656:186 | | | No: 792 | No: 1088 or | (AK125808) |
| | | | lambda-like | | | | | SEQ ID | |
| | | | polypeptide 1, pre-b- | | | | | No: 1089 | |
| | | | cell specific) | | | | | | |
| 95 | | image:156715 | | ughs.416139:186 | − | 0.0007 | SEQ ID | SEQ ID | SEQ ID No: 1120 |
| | | | | | | | No: 227 | No: 1060 | |
| 6 | S100A8 | image:1089513 | s100 calcium | ughs.416073:186 | − | 0.0008 | SEQ ID | | SEQ ID No: 12 |
| | | | binding protein a8 | | | | No: 11 | | (nm_002964) |
| | | | (calgranulin a) | | | | | | |
| 299 | PIM2 | image:46959 | pim-2 oncogene | ughs.496096:186 | − | 0.0009 | SEQ ID | SEQ ID No: 742 | SEQ ID No: 743 |
| | | | | | | | No: 741 | | (nm_006875) |
| 175 | TP53 | image:236338 | tumor protein p53 | ughs.408312:186 | − | 0.001 | SEQ ID | SEQ ID No: 413 | SEQ ID No: 414 |
| | | | (li-fraumeni | | | | No: 412 | | (nm_000546) |
| | | | syndrome) | | | | | | |
| 404 | ITGB3 | ipso:0000152 | integrin, beta 3 | ughs.218040:186 | − | 0.0012 | | SEQ ID No: 995 | SEQ ID No: 374 |
| | | | (platelet | | | | | | (nm_000212) |
| | | | glycoprotein iiia, | | | | | | |
| | | | antigen cd61) | | | | | | |
| 287 | LAMB1 | image:428443 | laminin, beta 1 | ughs.489646:186 | − | 0.0012 | SEQ ID | SEQ ID No: 710 | SEQ ID No: 711 |
| | | | | | | | No: 709 | | (nm_002291) |
| 269 | SILV | image:342383 | silver homolog | ughs.95972:186 | − | 0.0012 | SEQ ID | SEQ ID No: 662 | SEQ ID No: 663 |
| | | | (mouse) | | | | No: 661 | | (nm_006928) |
| 392 | | ipso:0000040 | cdna flj42596 fis, | ughs.113271:186 | − | 0.0012 | | SEQ ID No: 977 | SEQ ID No: 1102 |
| | | | clone brace3010283 | | | | | | (AK124587) |
| 100 | PIGR | image:159410 | polymeric | ughs.497589:186 | − | 0.0012 | SEQ ID | | SEQ ID No: 237 |
| | | | immunoglobulin | | | | No: 236 | | (nm_002644) |
| | | | receptor | | | | | | |
| 25 | CSH1 | image:133891 | chorionic | ughs.347963:186 | − | 0.0016 | | SEQ ID No: 59 | SEQ ID No: 60 |
| | | | somatomammotropin | | | | | | (nm_022640) |
| | | | hormone 1 | | | | | | |
| | | | (placental lactogen) | | | | | | |
| 153 | RDX | image:193081 | radixin | ughs.263671:186 | − | 0.0018 | SEQ ID | SEQ ID No: 360 | SEQ ID No: 361 |
| | | | | | | | No: 359 | | (nm_002906) |
| 49 | ETF1/FLT1 | image:146976 | eukaryotic | ughs.483494:186; | − | 0.0019 | SEQ ID | | SEQ ID No: 119 |
| | | | translation | ughs.507621:186 | | | No: 118 | | (nm_004730) or |
| | | | termination factor | | | | | | SEQ ID No: 1109 |
| | | | 1/fms-related | | | | | | (NM_002019) |
| | | | tyrosine kinase 1 | | | | | | |
| 69 | PFKP | image:152714 | phosphofructokinase, | ughs.26010:186 | − | 0.0019 | SEQ ID | SEQ ID No: 166 | SEQ ID No: 167 |
| | | | platelet | | | | No: 165 | | (nm_002627) |
| 143 | CXORF38 | image:188005 | chromosome x open | ughs.495961:186 | − | 0.002 | SEQ ID | SEQ ID No: 338 | SEQ ID No: 339 |
| | | | reading frame 38 | | | | No: 337 | | (nm_144970) |
| 140 | MGC15606 | image:187120 | family with | ughs.130195:186 | − | 0.0021 | SEQ ID | SEQ ID No: 332 | SEQ ID No: 333 |
| | | | sequence similarity | | | | No: 331 | | (nm_145037) |
| | | | 55, member c | | | | | | |
| 402 | ITGB3 | ipso:0000143 | integrin, beta 3 | ughs.218040:186 | − | 0.0022 | | SEQ ID No: 992 | SEQ ID No: 374 |
| | | | (platelet | | | | | | (nm_000212) |
| | | | glycoprotein iiia, | | | | | | |
| | | | antigen cd61) | | | | | | |
| 34 | SLAC2-B | image:142546 | slac2-b | N_A | − | 0.0024 | | SEQ ID No: 82 | SEQ ID No: 83 |
| | | | | | | | | | (nm_015065) |
| 139 | FLJ10986 | image:187119 | hypothetical protein | ughs.444301:186; | − | 0.0026 | SEQ ID | SEQ ID No: 329 | SEQ ID No: 330 |
| | | | flj10986 | ughs.439112:186 | | | No: 328 | | (nm_018291) |
| 421 | SERPINB1 | ipso:0000605 | serine (or cysteine) | ughs.381167:186 | − | 0.0037 | | SEQ ID | SEQ ID No: 1024 |
| | | | proteinase inhibitor, | | | | | No: 1023 | (nm_030666) |
| | | | clade b (ovalbumin), | | | | | | |
| | | | member 1 | | | | | | |
| 96 | RPS6KA3 | image:156808 | ribosomal protein s6 | ughs.445387:186 | − | 0.0048 | | SEQ ID No: 228 | SEQ ID No: 229 |
| | | | kinase, 90 kda, | | | | | | (nm_004586) |
| | | | polypeptide 3 | | | | | | |
| 370 | GATA6 | image:771332 | gata binding protein 6 | ughs.514746:186 | − | 0.0049 | SEQ ID | SEQ ID No: 924 | SEQ ID No: 925 |
| | | | | | | | No: 923 | | (nm_005257) |
| 316 | MTIF2 | image:50754 | mitochondrial | ughs.149894:186 | − | 0.0054 | SEQ ID | | SEQ ID No: 788 |
| | | | translational | | | | No: 787 | | (nm_001005369) |
| | | | initiation factor 2 | | | | | | |
| 413 | | ipso:0000376 | | N_A | − | 0.0057 | | SEQ ID | SEQ ID No: 1104 |
| | | | | | | | | No: 1010 | (AK128524) |
| 397 | | ipso:0000119 | | N_A | − | 0.0064 | | SEQ ID No: 985 | SEQ ID No: 1103 |
| | | | | | | | | | (BX108410) |
| 27 | IFNGR1 | image:136478 | interferon gamma | ughs.520414:186 | − | 0.0066 | SEQ ID | SEQ ID No: 65 | SEQ ID No: 66 |
| | | | receptor 1 | | | | No: 64 | | (nm_000416) |
| 425 | EBF | ipso:0000617 | early b-cell factor | ughs.308048:186 | − | 0.0067 | | SEQ ID | SEQ ID No: 1030 |
| | | | | | | | | No: 1029 | (nm_024007) |
| 92 | | image:156283 | | N_A | − | 0.0073 | SEQ ID | SEQ ID No: 222 | SEQ ID No: 1119 |
| | | | | | | | No: 221 | | |
| 148 | p66alpha | image:188422 | GATA zinc finger | ughs.551742:186 | − | 0.0074 | SEQ ID | | SEQ ID No: 1068 |
| | | | domain containing | | | | No: 350 | | (AK024670) |
| | | | 2A (p66alpha) | | | | | | |
| 102 | FKBP1A | image:159521 | fk506 binding | ughs.471933:186 | − | 0.0089 | SEQ ID | SEQ ID No: 240 | SEQ ID No: 241 |
| | | | protein 1a, 12 kda | | | | No: 239 | | (nm_000801) |
| 168 | SNAPC3 | image:219829 | small nuclear rna | ughs.546299:186 | − | 0.0089 | SEQ ID | SEQ ID No: 397 | SEQ ID No: 398 |
| | | | activating complex, | | | | No: 396 | | (nm_003084) |
| | | | polypeptide 3, | | | | | | |
| | | | 50 kda | | | | | | |
| 159 | ITGB3 | image:200209 | integrin, beta 3 | ughs.218040:186 | − | 0.0097 | SEQ ID | | SEQ ID No: 374 |
| | | | (platelet | | | | No: 373 | | (nm_000212) |
| | | | glycoprotein iiia, | | | | | | |
| | | | antigen cd61) | | | | | | |
| 30 | IL2RB | image:139073 | interleukin 2 | ughs.474787:186; | − | 0.0097 | SEQ ID | SEQ ID No: 73 | SEQ ID No: 74 |
| | | | receptor, beta | ughs.555488:186 | | | No: 72 | | (nm_000878) |
| 313 | | image:50541 | Homo sapiens | ughs.535157:186 | − | 0.0097 | SEQ ID | SEQ ID No: 780 | SEQ ID No: 1087 |
| | | | mRNA for FLJ00204 | | | | No: 779 | | (AK074131) |
| | | | protein | | | | | | |
| 381 | ETV4 | image:809959 | ets variant gene 4 | ughs.434059:186 | − | 0.01 | SEQ ID | SEQ ID No: 954 | SEQ ID No: 955 |
| | | | (e1a enhancer | | | | No: 953 | | (nm_001986) |
| | | | binding protein, | | | | | | |
| | | | e1af) | | | | | | |
| 29 | IL1R2 | image:137575 | interleukin 1 | ughs.25333:186 | − | 0.0101 | SEQ ID | SEQ ID No: 70 | SEQ ID No: 71 |
| | | | receptor, type ii | | | | No: 69 | | (nm_004633) |
| 416 | IGHG1 | ipso:0000434 | immunoglobulin | ughs.510635:186 | − | 0.0104 | | SEQ ID | SEQ ID No: 1105 |
| | | | heavy constant | | | | | No: 1015 | (BC072392) |
| | | | gamma 1 (g1m | | | | | | |
| | | | marker) | | | | | | |
| 343 | LCN2 | image:544683 | lipocalin 2 | ughs.204238:186 | − | 0.0107 | SEQ ID | SEQ ID No: 855 | SEQ ID No: 856 |
| | | | (oncogene 24p3) | | | | No: 854 | | (nm_005564) |
| 97 | CMRF35 | image:156937 | cd300c antigen | ughs.2605:186 | − | 0.0112 | | SEQ ID No: 230 | SEQ ID No: 231 |
| | | | | | | | | | (nm_006678) |
| 244 | CXCL1 | image:323238 | chemokine (c—x—c | ughs.789:186 | − | 0.0117 | SEQ ID | SEQ ID No: 592 | SEQ ID No: 593 |
| | | | motif) ligand 1 | | | | No: 591 | | (nm_001511) |
| | | | (melanoma growth | | | | | | |
| | | | stimulating activity, | | | | | | |
| | | | alpha) | | | | | | |
| 353 | MYBL2 | image:724259 | v-myb | ughs.179718:186 | − | 0.0122 | SEQ ID | SEQ ID No: 880 | SEQ ID No: 384 |
| | | | myeloblastosis viral | | | | No: 879 | | (nm_002466) |
| | | | oncogene homolog | | | | | | |
| | | | (avian)-like 2 | | | | | | |
| 216 | SLAMF8 | image:288807 | slam family member 8 | ughs.438683:186 | − | 0.0131 | SEQ ID | SEQ ID No: 518 | SEQ ID No: 519 |
| | | | | | | | No: 517 | | (nm_020125) |
| 239 | CTSC | image:320656 | cathepsin c | ughs.128065:186 | − | 0.016 | SEQ ID | SEQ ID No: 578 | SEQ ID No: 579 |
| | | | | | | | No: 577 | | (nm_001814) |
| 430 | ENPP2 | ipso:0000727 | ectonucleotide | ughs.190977:186 | − | 0.0205 | | SEQ ID | SEQ ID No: 1039 |
| | | | pyrophosphatase/ | | | | | No: 1038 | (nm_006209) |
| | | | phosphodiesterase 2 | | | | | | |
| | | | (autotaxin) | | | | | | |
| 14 | LAD1 | image:121551 | ladinin 1 | ughs.519035:186 | − | 0.021 | SEQ ID | SEQ ID No: 30 | SEQ ID No: 31 |
| | | | | | | | No: 29 | | (nm_005558) |
| 138 | RABL3 | image:186926 | rab, member of ras | ughs.444360:186; | − | 0.0221 | | SEQ ID No: 326 | SEQ ID No: 327 |
| | | | oncogene family-like 3 | ughs.548087:186 | | | | | (nm_173825) |
| 237 | HDAC2 | image:309924 | histone deacetylase 2 | ughs.3352:186 | − | 0.0243 | SEQ ID | SEQ ID No: 572 | SEQ ID No: 573 |
| | | | | | | | No: 571 | | (nm_001527) |
| 40 | VGLL1 | image:143622 | vestigial like 1 | N_A | − | 0.0245 | SEQ ID | SEQ ID No: 97 | SEQ ID No: 98 |
| | | | (drosophila) | | | | No: 96 | | (nm_016267) |
| 94 | npc-a-5 | image:156691 | nasopharyngeal | ughs.510543:186 | − | 0.0259 | SEQ ID | SEQ ID | SEQ ID No: 1059 |
| | | | carcinoma- | | | | No: 226 | No: 1058 | (AK091113) |
| | | | associated antigen | | | | | | |
| | | | npc-a-5 | | | | | | |
| 355 | CDK4 | image:725349 | cyclin-dependent | ughs.95577:186 | − | 0.0262 | SEQ ID | SEQ ID No: 885 | SEQ ID No: 886 |
| | | | kinase 4 | | | | No: 884 | | (nm_000075) |
| 426 | ABCC5 | ipso:0000654 | atp-binding | ughs.368563:186 | − | 0.0262 | | SEQ ID | SEQ ID No: 1032 |
| | | | cassette, sub-family | | | | | No: 1031 | (nm_005688) |
| | | | c (cftr/mrp), member 5 | | | | | | |
| 319 | MGC9913 | image:50892 | hypothetical protein | ughs.23133:186 | − | 0.0271 | SEQ ID | SEQ ID No: 794 | SEQ ID No: 1091 |
| | | | mgc9913 | | | | No: 793 | | (XM_378178) |
| 98 | FUT8 | image:156966 | fucosyltransferase 8 | ughs.118722:186 | − | 0.0283 | SEQ ID | | SEQ ID No: 233 |
| | | | (alpha (1,6) | | | | No: 232 | | (nm_178155) |
| | | | fucosyltransferase) | | | | | | |
| 375 | SFRP1 | image:783700 | secreted frizzled- | ughs.213424:186 | − | 0.0301 | SEQ ID | | SEQ ID No: 938 |
| | | | related protein 1 | | | | No: 937 | | (nm_003012) |
| 112 | ARPC2 | image:162208 | actin related protein | ughs.529303:186 | − | 0.0324 | SEQ ID | | SEQ ID No: 264 |
| | | | 2/3 complex, subunit | | | | No: 263 | | (nm_152862) |
| | | | 2, 34 kda | | | | | | |
| 227 | LILRB2 | image:30470 | leukocyte | ughs.534386:186 | − | 0.0329 | SEQ ID | SEQ ID No: 545 | SEQ ID No: 546 |
| | | | immunoglobulin-like | | | | No: 544 | | (nm_005874) |
| | | | receptor, subfamily | | | | | | |
| | | | b (with tm and itim | | | | | | |
| | | | domains), member 2 | | | | | | |
| 350 | IGKC | image:713852 | immunoglobulin | ughs.449621:186; | − | 0.0346 | SEQ ID | SEQ ID | SEQ ID No: 1099 |
| | | | kappa constant | ughs.546620:186 | | | No: 872 | No: 1097 or | (BC066343) |
| | | | | | | | | SEQ ID | |
| | | | | | | | | No: 1098 | |
| 429 | SN | ipso:0000704 | sialoadhesin | ughs.31869:186 | − | 0.0377 | | SEQ ID | SEQ ID No: 1037 |
| | | | | | | | | No: 1036 | (nm_023068) |
| 229 | C1ORF38 | image:307255 | chromosome 1 open | ughs.10649:186 | − | 0.0378 | SEQ ID | SEQ ID No: 549 | SEQ ID No: 550 |
| | | | reading frame 38 | | | | No: 548 | | (nm_004848) |
| 423 | PADI2 | ipso:0000610 | peptidyl arginine | ughs.33455:186 | − | 0.0418 | | SEQ ID | SEQ ID No: 1027 |
| | | | deiminase, type ii | | | | | No: 1026 | (nm_007365) |
| 410 | MONDOA | ipso:0000314 | mlx interactor | ughs.437153:186 | − | 0.0455 | | SEQ ID | SEQ ID No: 1005 |
| | | | | | | | | No: 1004 | (nm_014938) |
| 329 | TAP1 | image:51782 | transporter 1, atp- | ughs.352018:186; | − | 0.0458 | SEQ ID | SEQ ID No: 819 | SEQ ID No: 820 |
| | | | binding cassette, | ughs.552165:186 | | | No: 818 | | (nm_000593) |
| | | | sub-family b | | | | | | |
| | | | (mdr/tap) | | | | | | |
| 157 | CYP2D6 | image:199680 | cytochrome p450, | ughs.534311:186 | − | 0.047 | | SEQ ID No: 369 | SEQ ID No: 370 |
| | | | family 2, subfamily | | | | | | (nm_000106) |
| | | | d, polypeptide 6 | | | | | | |

TABLE VII
Metagene underEGFR

| | | | | | Reg- | | | | |
|---|---|---|---|---|---|---|---|---|---|
| Set | Gene | | | | ula- | | | | |
| No. | symbol | Clone ID | Gene name | Unigene Cluster | tion | P value | Seq3′ | Seq5′ | Ref. Seq |
| 197 | | image:266500 | LOC255743: | N_A | − | 1E−05 | SEQ ID | | SEQ ID No: 1071 |
| | | | Nephronectin | | | | No: 472 | | (NM_001033047) |
| 107 | LU | image:160656 | lutheran blood group | ughs.155048:186 | − | 1E−05 | SEQ ID | | SEQ ID No: 254 |
| | | | (auberger b antigen | | | | No: 253 | | (nm_005581) |
| | | | included) | | | | | | |
| 3 | TFF1 | image:1075949 | trefoil factor 1 | ughs.162807:186 | − | 1E−05 | SEQ ID | | SEQ ID No: 6 |
| | | | (breast cancer, | | | | No: 5 | | (nm_003225) |
| | | | estrogen-inducible | | | | | | |
| | | | sequence | | | | | | |
| | | | expressed in) | | | | | | |
| 354 | ESR1 | image:725321 | estrogen receptor 1 | ughs.208124:186 | − | 1E−05 | SEQ ID | SEQ ID No: 882 | SEQ ID No: 883 |
| | | | | | | | No: 881 | | (nm_000125) |
| 226 | XBP1 | image:301950 | x-box binding | ughs.437638:186 | − | 1E−05 | SEQ ID | SEQ ID No: 542 | SEQ ID No: 543 |
| | | | protein 1 | | | | No: 541 | | (nm_005080) |
| 275 | SCUBE2 | image:346321 | signal peptide, cub | ughs.523468:186 | − | 1E−05 | SEQ ID | SEQ ID No: 680 | SEQ ID No: 681 |
| | | | domain, egf-like 2 | | | | No: 679 | | (nm_020974) |
| 26 | GATA3 | image:135118 | gata binding protein 3 | ughs.524134:186 | − | 1E−05 | SEQ ID | SEQ ID No: 62 | SEQ ID No: 63 |
| | | | | | | | No: 61 | | (nm_001002295) |
| 31 | GATA3 | image:139076 | gata binding protein 3 | ughs.524134:186 | − | 1E−05 | SEQ ID | SEQ ID No: 76 | SEQ ID No: 63 |
| | | | | | | | No: 75 | | (nm_001002295) |
| 88 | EIF2C3 | image:155341 | eukaryotic | ughs.530333:186 | − | 1E−05 | | SEQ ID No: 211 | SEQ ID No: 212 |
| | | | translation initiation | | | | | | (nm_024852) |
| | | | factor 2c, 3 | | | | | | |
| 309 | C4A | image:491004 | complement | ughs.534847:186 | − | 1E−05 | SEQ ID | SEQ ID No: 771 | SEQ ID No: 635 |
| | | | component 4b, | | | | No: 770 | | (nm_001002029) |
| | | | telomeric | | | | | | |
| 223 | TFF3 | image:298417 | trefoil factor 3 | ughs.82961:186 | − | 1E−05 | SEQ ID | | SEQ ID No: 535 |
| | | | (intestinal) | | | | No: 534 | | (nm_003226) |
| 424 | | ipso:0000614 | | N_A | − | 3E−05 | | SEQ ID | SEQ ID No: 1125 |
| | | | | | | | | No: 1028 | |
| 44 | NAT1 | image:145894 | n-acetyltransferase | ughs.155956:186 | − | 3E−05 | SEQ ID | SEQ ID No: 108 | SEQ ID No: 109 |
| | | | 1 (arylamine n- | | | | No: 107 | | (nm_000662) |
| | | | acetyltransferase) | | | | | | |
| 144 | COL4A2 | image:188193 | collagen, type iv, | ughs.508716:186 | − | 3E−05 | SEQ ID | SEQ ID No: 341 | SEQ ID No: 342 |
| | | | alpha 2 | | | | No: 340 | | (nm_001846) |
| 259 | C4A | image:340753 | complement | ughs.534847:186 | − | 3E−05 | SEQ ID | SEQ ID No: 634 | SEQ ID No: 635 |
| | | | component 4b, | | | | No: 633 | | (nm_001002029) |
| | | | telomeric | | | | | | |
| 371 | RABEP1 | image:772890 | rabaptin, rab gtpase | ughs.551518:186 | − | 3E−05 | SEQ ID | | SEQ ID No: 927 |
| | | | binding effector | | | | No: 926 | | (nm_004703) |
| | | | protein 1 | | | | | | |
| 398 | | ipso:0000125 | | N_A | − | 5E−05 | | SEQ ID No: 986 | SEQ ID No: 1124 |
| 51 | RHOBTB3 | image:147138 | rho-related btb | ughs.445030:186 | − | 6E−05 | SEQ ID | SEQ ID No: 123 | SEQ ID No: 124 |
| | | | domain containing 3 | | | | No: 122 | | (nm_014899) |
| 119 | CASKI | image:166862 | cask interacting | ughs.530863:186; | − | 6E−05 | SEQ ID | | SEQ ID No: 280 |
| | N1/flj12650 | | protein 1 | ughs.470259:186 | | | No: 279 | | (nm_020764) or |
| | | | | | | | | | SEQ ID No: 1110 |
| | | | | | | | | | (NM_024522) |
| 126 | CXXC5 | image:173797 | cxxc finger 5 | ughs.189119:186 | − | 9E−05 | SEQ ID | SEQ ID No: 296 | SEQ ID No: 297 |
| | | | | | | | No: 295 | | (nm_016463) |
| 317 | MAPT | image:50764 | microtubule- | ughs.101174:186 | − | 0.0001 | SEQ ID | SEQ ID No: 790 | SEQ ID No: 791 |
| | | | associated protein | | | | No: 789 | | (nm_016835) |
| | | | tau | | | | | | |
| 87 | MGC24047 | image:155072 | chromosome 1 open | ughs.29190:186 | − | 0.0001 | SEQ ID | SEQ ID No: 209 | SEQ ID No: 210 |
| | | | reading frame 64 | | | | No: 208 | | (nm_178840) |
| 332 | MGC45441 | image:52118 | hypothetical protein | ughs.488337:186 | − | 0.0003 | SEQ ID | | SEQ ID No: 827 |
| | | | mgc45441 | | | | No: 826 | | (nm_152499) |
| 135 | CYP2B6 | image:182295 | Cytochrome P450, | N_A | − | 0.0007 | SEQ ID | SEQ ID No: 320 | SEQ ID No: 1064 |
| | | | family 2, subfamily | | | | No: 319 | | (NM_000767) |
| | | | B, polypeptide 6 | | | | | | |
| 61 | CROCC | image:149567 | ciliary rootlet coiled- | ughs.309403:186; | − | 0.0007 | | SEQ ID No: 146 | SEQ ID No: 147 |
| | | | coil, rootletin | ughs.135718:186 | | | | | (nm_014675) |
| 136 | USP21 | image:183062 | ubiquitin specific | ughs.8015:186 | − | 0.0008 | SEQ ID | SEQ ID No: 322 | SEQ ID No: 323 |
| | | | protease 21 | | | | No: 321 | | (nm_001014443) |
| 43 | TRAF5 | image:145410 | tnf receptor- | ughs.523930:186 | − | 0.0011 | SEQ ID | SEQ ID No: 105 | SEQ ID No: 106 |
| | | | associated factor 5 | | | | No: 104 | | (nm_004619) |
| 75 | GSTM2 | image:153444 | glutathione s- | ughs.279837:186 | − | 0.0013 | | SEQ ID No: 180 | SEQ ID No: 181 |
| | | | transferase m2 | | | | | | (nm_000848) |
| | | | (muscle) | | | | | | |
| 160 | DUSP4 | image:2044325 | dual specificity | ughs.417962:186 | − | 0.0015 | SEQ ID | | SEQ ID No: 376 |
| | | | phosphatase 4 | | | | No: 375 | | (nm_057158) |
| 47 | ASF1A | image:146634 | asf1 anti-silencing | ughs.292316:186 | − | 0.0018 | SEQ ID | | SEQ ID No: 116 |
| | | | function 1 homolog | | | | No: 115 | | (nm_014034) |
| | | | a (s. cerevisiae) | | | | | | |
| 106 | CSF2 | image:1601601 | colony stimulating | ughs.1349:186 | − | 0.0024 | SEQ ID | | SEQ ID No: 252 |
| | | | factor 2 | | | | No: 251 | | (nm_000758) |
| | | | (granulocyte- | | | | | | |
| | | | macrophage) | | | | | | |
| 320 | CLSTN2 | image:50970 | calsyntenin 2 | ughs.158529:186 | − | 0.0025 | SEQ ID | SEQ ID No: 796 | SEQ ID No: 797 |
| | | | | | | | No: 795 | | (nm_022131) |
| 365 | GLI3 | image:767495 | gli-kruppel family | ughs.199338:186 | − | 0.0028 | SEQ ID | | SEQ ID No: 911 |
| | | | member gli3 (greig | | | | No: 910 | | (nm_000168) |
| | | | cephalopolysyndactyly | | | | | | |
| | | | syndrome) | | | | | | |
| 291 | REPS2 | image:43488 | ralbp1 associated | ughs.186810:186; | − | 0.0031 | SEQ ID | SEQ ID No: 719 | SEQ ID No: 720 |
| | | | eps domain | ughs.131188:186 | | | No: 718 | | (nm_004726) |
| | | | containing 2 | | | | | | |
| 356 | GSTM1 | image:73778 | glutathione s- | ughs.301961:186 | − | 0.0031 | SEQ ID | SEQ ID No: 888 | SEQ ID No: 889 |
| | | | transferase m1 | | | | No: 887 | | (nm_000561) |
| 407 | PLAT | ipso:0000253 | plasminogen | ughs.491582:186 | − | 0.0034 | | SEQ ID | SEQ ID No: 250 |
| | | | activator, tissue | | | | | No: 1000 | (nm_000930) |
| 74 | DLG5 | image:153368 | discs, large homolog | ughs.500245:186 | − | 0.0039 | SEQ ID | | SEQ ID No: 179 |
| | | | 5 (drosophila) | | | | No: 178 | | (nm_004747) |
| 315 | FLJ00012 | image:50602 | flj00012 protein | ughs.21051:186 | − | 0.004 | SEQ ID | SEQ ID No: 785 | SEQ ID No: 786 |
| | | | | | | | No: 784 | | (nm_033388) |
| 73 | SIDT2 | image:153205 | sid1 transmembrane | ughs.410977:186 | − | 0.0041 | SEQ ID | | SEQ ID No: 177 |
| | | | family, member 2 | | | | No: 176 | | (nm_015996) |
| 39 | | image:143169 | | N_A | − | 0.0043 | SEQ ID | SEQ ID | SEQ ID No: 1047 |
| | | | | | | | No: 95 | No: 1046 | (BC012900) |
| 128 | BCL9 | image:1756392 | b-cell cll/lymphoma 9 | ughs.415209:186 | − | 0.0043 | SEQ ID | | SEQ ID No: 301 |
| | | | | | | | No: 300 | | (nm_004326) |
| 86 | USP13 | image:155064 | ubiquitin specific | ughs.175322:186 | − | 0.0052 | SEQ ID | SEQ ID No: 206 | SEQ ID No: 207 |
| | | | protease 13 | | | | No: 205 | | (nm_003940) |
| | | | (isopeptidase t-3) | | | | | | |
| 374 | DNALI1 | image:782688 | dynein, axonemal, | ughs.406050:186 | − | 0.0061 | SEQ ID | SEQ ID No: 935 | SEQ ID No: 936 |
| | | | light intermediate | | | | No: 934 | | (nm_003462) |
| | | | polypeptide 1 | | | | | | |
| 367 | FOXC1/ | image:768370 | forkhead box c1/ras | ughs.348883:186; | − | 0.0065 | | SEQ ID No: 915 | SEQ ID No: 916 |
| | RHOB | | homolog gene | ughs.502876:186 | | | | | (nm_001453) or |
| | | | | | | | | | SEQ ID No: 1116 |
| | | | | | | | | | (NM_004040) |
| 62 | | image:149760 | | N_A | − | 0.007 | SEQ ID | SEQ ID No: 149 | SEQ ID No: 1052 |
| | | | | | | | No: 148 | | (BX096026) |
| 345 | GSTM2 | image:664233 | glutathione s- | ughs.279837:186 | − | 0.0079 | SEQ ID | SEQ ID No: 860 | SEQ ID No: 181 |
| | | | transferase m2 | | | | No: 859 | | (nm_000848) |
| | | | (muscle) | | | | | | |
| 66 | KRT18 | image:151663 | keratin 18 | ughs.406013:186 | − | 0.0088 | SEQ ID | SEQ ID No: 158 | SEQ ID No: 159 |
| | | | | | | | No: 157 | | (nm_000224) |
| 340 | | image:52898 | | ughs.548040:186 | − | 0.0089 | SEQ ID | SEQ ID | SEQ ID No: 1096 |
| | | | | | | | No: 847 | No: 1094 or | (AK127274) |
| | | | | | | | | SEQ ID | |
| | | | | | | | | No: 1095 | |
| 13 | DNAJC12 | image:120138 | dnaj (hsp40) | ughs.260720:186 | − | 0.0094 | SEQ ID | SEQ ID No: 27 | SEQ ID No: 28 |
| | | | homolog, subfamily | | | | No: 26 | | (nm_021800) |
| | | | c, member 12 | | | | | | |
| 77 | | image:153617 | cdna flj41270 fis, | ughs.445414:186 | − | 0.0096 | SEQ ID | SEQ ID No: 185 | SEQ ID No: 1054 |
| | | | clone | | | | No: 184 | | (AK123264) |
| | | | bramy2036387 | | | | | | |
| 12 | SPDEF/ | image:1188588 | sam pointed domain | ughs.124299:186; | − | 0.0098 | SEQ ID | SEQ ID No: 24 | SEQ ID No: 25 |
| | c8orf13 | | containing ets | ughs.485158:186 | | | No: 23 | | (nm_012391) or |
| | | | transcription factor/ | | | | | | SEQ ID No: 1108 |
| | | | chromosome 8 open | | | | | | (NM_053279) |
| | | | reading frame 13 | | | | | | |
| 331 | C20ORF23 | image:52103 | chromosome 20 | ughs.101774:186 | − | 0.0102 | SEQ ID | | SEQ ID No: 825 |
| | | | open reading frame | | | | No: 824 | | (nm_024704) |
| | | | 23 | | | | | | |
| 60 | FLJ20366 | image:149549 | hypothetical protein | ughs.390738:186 | − | 0.0128 | SEQ ID | SEQ ID No: 144 | SEQ ID No: 145 |
| | | | flj20366 | | | | No: 143 | | (nm_017786) |
| 204 | COX6C | image:278531 | cytochrome c | ughs.351875:186 | − | 0.014 | SEQ ID | SEQ ID No: 490 | SEQ ID No: 491 |
| | | | oxidase subunit vic | | | | No: 489 | | (nm_004374) |
| 202 | RGS11 | image:277917 | regulator of | ughs.65756:186 | − | 0.0142 | | SEQ ID No: 484 | SEQ ID No: 485 |
| | | | g-protein signalling | | | | | | (nm_003834) |
| | | | 11 | | | | | | |
| 206 | | image:280743 | Hypothetical protein | ughs.508559:186 | − | 0.0148 | SEQ ID | SEQ ID No: 496 | SEQ ID No: 1072 |
| | | | LOC153561 | | | | No: 495 | | (AY007114) |
| 116 | SEMA6B | image:166010 | sema domain, | ughs.465642:186 | − | 0.0157 | SEQ ID | SEQ ID No: 273 | SEQ ID No: 274 |
| | | | transmembrane | | | | No: 272 | | (nm_032108) |
| | | | domain (tm), and | | | | | | |
| | | | cytoplasmic domain, | | | | | | |
| | | | (semaphorin) 6b | | | | | | |
| 109 | AP1G2 | image:161763 | adaptor-related | ughs.343244:186 | − | 0.0171 | SEQ ID | SEQ ID No: 257 | SEQ ID No: 258 |
| | | | protein complex 1, | | | | No: 256 | | (nm_080545) |
| | | | gamma 2 subunit | | | | | | |
| 124 | AKAP8L | image:171679 | a kinase (prka) | ughs.399800:186 | − | 0.0182 | SEQ ID | SEQ ID No: 291 | SEQ ID No: 292 |
| | | | anchor protein 8-like | | | | No: 290 | | (nm_014371) |
| 322 | PRKCBP1 | image:511899 | protein kinase c | ughs.446240:186 | − | 0.0184 | SEQ ID | SEQ ID No: 802 | SEQ ID No: 803 |
| | | | binding protein 1 | | | | No: 801 | | (nm_183047) |
| 120 | GSTM2 | image:166910 | glutathione s- | ughs.279837:186 | − | 0.0186 | SEQ ID | SEQ ID No: 282 | SEQ ID No: 181 |
| | | | transferase m2 | | | | No: 281 | | (nm_000848) |
| | | | (muscle) | | | | | | |
| 105 | PLAT | image:160149 | plasminogen | ughs.491582:186 | − | 0.0196 | SEQ ID | SEQ ID No: 249 | SEQ ID No: 250 |
| | | | activator, tissue | | | | No: 248 | | (nm_000930) |
| 147 | CENTG3 | image:188414 | centaurin, gamma 3 | ughs.195048:186 | − | 0.0205 | SEQ ID | SEQ ID No: 348 | SEQ ID No: 349 |
| | | | | | | | No: 347 | | (nm_031946) |
| 339 | | image:52870 | genomic region on | ughs.159853:186 | − | 0.0246 | SEQ ID | SEQ ID No: 846 | SEQ ID No: 1123 |
| | | | chromosome 1 | | | | No: 1093 | or SEQ ID | |
| | | | | | | | | No: 1092 | |
| 306 | SLC40A1 | image:489218 | solute carrier family | ughs.529285:186 | − | 0.0246 | SEQ ID | SEQ ID No: 762 | SEQ ID No: 763 |
| | | | 40 (iron-regulated | | | | No: 761 | | (nm_014585) |
| | | | transporter), | | | | | | |
| | | | member 1 | | | | | | |
| 183 | CCND2 | image:249688 | cyclin d2 | ughs.376071:186 | − | 0.0272 | SEQ ID | SEQ ID No: 437 | SEQ ID No: 438 |
| | | | | | | | No: 436 | | (nm_001759) |
| 38 | KLHDC2 | image:143060 | kelch domain | N_A | − | 0.028 | SEQ ID | SEQ ID No: 93 | SEQ ID No: 94 |
| | | | containing 2 | | | | No: 92 | | (nm_014315) |
| 338 | ABCA3 | image:52741 | atp-binding | ughs.26630:186 | − | 0.0344 | SEQ ID | SEQ ID No: 844 | SEQ ID No: 845 |
| | | | cassette, sub-family | | | | No: 843 | | (nm_001089) |
| | | | a (abc1), member 3 | | | | | | |
| 293 | LOC143381 | image:44338 | hypothetical protein | ughs.388347:186; | − | 0.0371 | SEQ ID | SEQ ID No: 725 | SEQ ID No: 1084 |
| | | | loc143381 | ughs.557061:186 | | | No: 724 | | (BX648964) |
| 296 | FLJ21439 | image:45814 | hypothetical protein | ughs.550536:186 | − | 0.0375 | SEQ ID | SEQ ID No: 733 | SEQ ID No: 734 |
| | | | flj21439 | | | | No: 732 | | (nm_025137) |
| 377 | HOXA4 | image:785930 | homeo box a4 | ughs.77637:186 | − | 0.039 | SEQ ID | SEQ ID No: 942 | SEQ ID No: 943 |
| | | | | | | | No: 941 | | (nm_002141) |
| 311 | CACNA1D/ | image:49630 | Calcium channel, | ughs.476358:186; | − | 0.0396 | SEQ ID | SEQ ID | SEQ ID No: 1085 |
| | KIF5C | | voltage-dependent, | ughs.435557:186 | | | No: 775 | No: 1086 | (NM_000720) |
| | | | L type, alpha 1D | | | | | | |
| | | | subunit/kinesin | | | | | | |
| | | | famillly member 5c | | | | | | |
| 117 | GNG3 | image:166254 | guanine nucleotide | ughs.179915:186 | − | 0.0494 | | SEQ ID No: 275 | SEQ ID No: 276 |
| | | | binding protein (g | | | | | | (nm_012202) |
| | | | protein), gamma 3 | | | | | | |

TABLE VIII
Metagene overEGFR

| Set | Gene | | | Unigene | Regu- | | | | |
|---|---|---|---|---|---|---|---|---|---|
| No. | symbol | Clone ID | Gene name | Cluster | lation | P value | Seq3′ | Seq5′ | Ref. Seq |
| 171 | GSTP1 | image:231424 | glutathione s- | ughs.523836:186 | + | 0.00005 | SEQ ID | SEQ ID No: 404 | SEQ ID No: 405 |
| | | | transferase pi | | | | No: 403 | | (nm_000852) |
| 402 | ITGB3 | ipso:0000143 | integrin, beta 3 | ughs.218040:186 | + | 0.00008 | | SEQ ID No: 992 | SEQ ID No: 374 |
| | | | (platelet | | | | | | (nm_000212) |
| | | | glycoprotein iiia, | | | | | | |
| | | | antigen cd61) | | | | | | |
| 217 | IGHG1 | image:289337 | immunoglobulin | ughs.510635:186 | + | 0.00011 | SEQ ID | SEQ ID No: 521 | SEQ ID No: 1122 |
| | | | heavy constant | | | | No: 520 | | |
| | | | gamma 1 (g1m | | | | | | |
| | | | marker) | | | | | | |
| 246 | SOD2 | image:324014 | superoxide | ughs.487046:186 | + | 0.00072 | SEQ ID | | SEQ ID No: 598 |
| | | | dismutase 2, | | | | No: 597 | | (nm_000636) |
| | | | mitochondrial | | | | | | |
| 111 | CEBPB | image:161993 | ccaat/enhancer | ughs.517106:186 | + | 0.00089 | SEQ ID | | SEQ ID No: 262 |
| | | | binding protein | | | | No: 261 | | (nm_005194) |
| | | | (c/ebp), beta | | | | | | |
| 350 | IGKC | image:713852 | immunoglobulin | ughs.449621:186; | + | 0.00177 | SEQ ID | SEQ ID | SEQ ID No: 1099 |
| | | | kappa constant | ughs.546620:186 | | | No: 872 | No: 1097 or | (BC066343) |
| | | | | | | | | SEQ ID | |
| | | | | | | | | No: 1098 | |
| 282 | ENO1 | image:392678 | enolase 1, (alpha) | ughs.517145:186 | + | 0.00201 | SEQ ID | | SEQ ID No: 696 |
| | | | | | | | No: 695 | | (nm_001428) |
| 94 | npc-a-5 | image:156691 | nasopharyngeal | ughs.510543:186 | + | 0.00352 | SEQ ID | SEQ ID | SEQ ID No: 1059 |
| | | | carcinoma- | | | | No: 226 | No: 1058 | (AK091113) |
| | | | associated antigen | | | | | | |
| | | | npc-a-5 | | | | | | |
| 302 | MMP7 | image:471134 | matrix | ughs.2256:186 | + | 0.00698 | SEQ ID | SEQ ID No: 750 | SEQ ID No: 751 |
| | | | metalloproteinase 7 | | | | No: 749 | | (nm_002423) |
| | | | (matrilysin, uterine) | | | | | | |
| 142 | | image:187744 | | N_A | + | 0.01196 | | SEQ ID No: 336 | SEQ ID No: 1121 |
| 122 | MKI67 | image:1693709 | antigen identified by | ughs.80976:186 | + | 0.0122 | SEQ ID | | SEQ ID No: 286 |
| | | | monoclonal antibody | | | | No: 285 | | (nm_002417) |
| | | | ki-67 | | | | | | |
| 103 | ARHGEF1 | image:159568 | rho guanine | ughs.278186:186 | + | 0.01427 | SEQ ID | SEQ ID No: 243 | SEQ ID No: 244 |
| | | | nucleotide exchange | | | | No: 242 | | (nm_199002) |
| | | | factor (gef) 1 | | | | | | |
| 8 | ATF2 | image:110999 | activating | ughs.425104:186 | + | 0.0148 | SEQ ID | SEQ ID No: 17 | SEQ ID No: 18 |
| | | | transcription factor 2 | | | | No: 16 | | (nm_001880) |
| 50 | TFCP2L1 | image:1470131 | transcription factor | ughs.156471:186 | + | 0.0259 | SEQ ID | | SEQ ID No: 121 |
| | | | cp2-like 1 | | | | No: 120 | | (nm_014553) |
| 427 | IGKC | ipso:0000658 | immunoglobulin | N_A | + | 0.02767 | | SEQ ID | SEQ ID No: 1107 |
| | | | kappa variable 1-5 | | | | | No: 1033 | (BC073775) |
| | | | (IGKC) | | | | | | |
| 42 | PRSS12 | image:145310 | protease. serine, 12 | ughs.445857:186 | + | 0.03118 | SEQ ID | SEQ ID No: 102 | SEQ ID No: 103 |
| | | | (neurotrypsin, | | | | No: 101 | | (nm_003619) |
| | | | motopsin) | | | | | | |
| 84 | IGLC2 | image:154809 | immunoglobulin | ughs.449585:186 | + | 0.04077 | SEQ ID | SEQ ID No: 201 | SEQ ID No: 1118 |
| | | | lambda joining 3 | | | | No: 200 | | |
| 18 | CSF1 | image:124554 | colony stimulating | ughs.173894:186 | + | 0.0412 | SEQ ID | | SEQ ID No: 42 |
| | | | factor 1 | | | | No: 41 | | (nm_000757) |
| | | | (macrophage) | | | | | | |
| 145 | LOC114659 | image:188196 | SH3-domain GRB2- | ughs.406166:186; | + | 0.04453 | SEQ ID | SEQ ID | SEQ ID No: 1067 |
| | | | like pseudogene 1 | ughs.438861:186 | | | No: 343 | No: 1065 | (AK123784) |
| | | | | | | | (=SEQ ID | | |
| | | | | | | | No: 1066) | | |

### Example 5

### Use of Metagenes According to the Invention on an Affymetrix® Platform (GeneChip® Human Genome U133 Plus 2.0 Array)

We profiled 113 samples from the validation set on the Affymetrix® platform to evaluate agreement between the 2 platforms.

A mapping was performed to find the Affymetrix® probesets corresponding to the sequences comprised into the 3 metagenes, using standard sequence alignment (blast) algorithms.

For a given gene, several Image clones may exist, each of them covering a particular region of the gene, more commonly in the 3′ region. Affymetrix® probesets are also designed to target a specific region of a gene, of around 1000 nucleotides. Clone inserts and Affymetrix® targets do not necessarily overlap, even if the same gene is considered.

Given this information, there were two possibilities to find a correspondence between Discovery™ and Affymetrix® plateform:

i) sequence alignment of clone inserts and probesets against a Reference Sequence (ReSeq), which represents a specific gene, and selection of pairs (Clone, Probeset) with homologies to the same Refseq, even if the these sequences do not overlap;

ii) consider only pairs which overlap, assuming that signal may differ according to the region we focus on. This second approach was chosen to select Affymetrix® probe sets corresponding to the Discovery clones.

Raw data from Affymetrix® platform were first normalized using the RMA (Robust Multichip Average) method available in Bioconductor (Irizarry et al. 2 . . . ) (Affymetrix® package), then corrected to take into account the inter-platform effect and calculate the score for each sample. The data processing applied was the same as previously described on the Discovery™ platform for normalization and Metagenes calculation.

As an example, comparing sample classification into good or poor prognosis group on Discovery™ and Affymetrix® platform, we obtained 95% when using appropriate confidence interval around the threshold.

The following tables (IX to XIV) are examples of metagenes of the invention that may be used with an Affymetrix® platform according to the above described methods. For each metagene (IX to XIV), at least two, preferably five, most preferably ten or all of the markers listed, e.g., genes, or marker-derived polynucleotides, e.g., Affymetrix® Probe Sets, may be used to perform these methods. The sequences of the listed Affymetrix® Probe Sets are provided in the enclosed sequence listing and are also publicly available from internet, e.g., www.affymetrix.com. For example, these underER, underPR and underEGFR metagenes may be used in the above described method using a Cox regression analysis and the score S_{C}=a×underER+b×underPR+c×under EGFR, wherein “a” is comprised in the interval [−6.26; +0.49], “b” is comprised in the interval [−2.65; +0.29] and “c” is comprised in the interval [−6.69; +1.65]. For example the formula is: S_{C}=−2.90279×underER−1.47423×underPR−4.17198×under EGFR. Preferably, metagenes of tables IX to XI are used together one the one hand, and metagenes of tables XII to XIV are used together on the other hand.

The error on the score was integrated by calculating a confidence interval around the threshold, within which sample classification was considered non robust. Considering the score distribution Gaussian, we estimated the confidence interval around the threshold using standard deviation calculation method (estimated standard deviation of the population/√n).

The inventors have established that a woman having a score (S_{C}) of more than 0.16 have at least a double propensity of poor clinical outcome than a woman with a score (S_{C}) of less than 0.015.

TABLE IX
Metagene underER

| Affymetrix ® | | | | | Reference sequence | |
|---|---|---|---|---|---|---|
| Probe Set | Clone | Gene | symbol | Unigene Reference | (refseq) | genbank |
| 213094_at | image:259884 | G protein-coupled receptor 126 | GPR126 | hs.318894 | nm_001032394, | al033377 |
| | | | | | nm_001032395, | |
| | | | | | nm_020455, | |
| | | | | | nm_198569 | |
| 204259_at | image:471134 | matrix metallopeptidase | MMP7 | hs.2256 | nm_002423 | nm_002423 |
| | | 7_matrilysin, uterine_{—} | | | | |
| 204733_at | image:724109 | kallikrein 6_neurosin, zyme_{—} | KLK6 | hs.79361 | nm_001012964, | nm_002774 |
| | | | | | nm_001012965, | |
| | | | | | nm_001012966, | |
| | | | | | nm_002774 | |
| 203560_at | image:809588 | gamma-glutamyl | GGH | hs.78619 | nm_003878 | nm_003878 |
| | | hydrolase_conjugase, | | | | |
| | | folylpolygammaglutamyl | | | | |
| | | hydrolase_{—} | | | | |
| 202705_at | image:845594 | cyclin B2 | CCNB2 | hs.194698 | nm_004701 | nm_004701 |
| 227004_at | image:301018 | Cyclin-dependent kinase-like 5 | CDKL5 | hs.435570 | nm_003159 | ai611074 |
| 202967_at | image:345309 | glutathione S-transferase A4 | GSTA4 | hs.485557 | nm_001512 | nm_001512 |
| 218060_s_at | image:43457 | hypothetical protein FLJ13154 | FLJ13154 | hs.408702 | nm_024598 | nm_024598 |
| 201579_at | image:1028762 | FAT tumor suppressor homolog | FAT | hs.481371 | nm_005245 | nm_005245 |
| | | 1_Drosophila_{—} | | | | |
| 208370_s_at | ipso:0000077 | Down syndrome critical region | DSCR1 | hs.282326 | nm_004414, | nm_004414 |
| | | gene 1 | | | nm_203417, | |
| | | | | | nm_203418 | |
| 225565_at | image:147707 | CDNA FLJ34215 fis, clone | NA | hs.516646 | — | aa769455 |
| | | FCBBF3021985 | | | | |
| 217728_at | image:512420 | S100 calcium binding protein | S100A6 | hs.275243 | nm_014624 | nm_014624 |
| | | A6_calcyclin_{—} | | | | |
| 236449_at | image:51814 | Cystatin B_stefin B_{—} | CSTB | hs.695 | nm_000100 | ai885390 |
| 212501_at | image:161993 | CCAAT_enhancer binding | CEBPB | hs.517106 | nm_005194 | al564683 |
| | | protein_C_EBP_, beta | | | | |
| 201487_at | image:320656 | cathepsin C | CTSC | hs.128065 | nm_001814, | nm_001814 |
| | | | | | nm_148170 | |
| 203287_at | image:121551 | ladinin 1 | LAD1 | hs.519035 | nm_005558 | nm_005558 |
| 212531_at | image:544683 | lipocalin 2_oncogene 24p3_{—} | LCN2 | hs.204238 | nm_005564 | nm_005564 |
| 212397_at | image:193081 | radixin | RDX | hs.263671 | nm_002906 | al137751 |
| 202917_s_at | image:1089513 | S100 calcium binding protein | S100A8 | hs.416073 | nm_002964 | nm_002964 |
| | | A8_calgranulin A_{—} | | | | |
| 205487_s_at | image:143622 | vestigial like 1_Drosophila_{—} | VGLL1 | hs.496843 | nm_016267 | nm_016267 |
| 221477_s_at | image:324014 | hypothetical protein MGC5618 | MGC5618 | NA | — | bf575213 |
| 201037_at | image:152714 | phosphofructokinase, platelet | PFKP | hs.26010 | nm_002627 | nm_002627 |

TABLE X
Metagene underPR

| | | | | | Reference | |
|---|---|---|---|---|---|---|
| Affymetrix ® | | | | Unigene | Sequence | |
| Probe Set | Clone | Gene | symbol | reference | (refseq) | genbank |
| 201487_at | image:320656 | cathepsin C | CTSC | hs.128065 | nm_001814, | nm_001814 |
| | | | | | nm_148170 | |
| 203287_at | image:121551 | ladinin 1 | LAD1 | hs.519035 | nm_005558 | nm_005558 |
| 212531_at | image:544683 | lipocalin 2_oncogene 24p3_{—} | LCN2 | hs.204238 | nm_005564 | nm_005564 |
| 212397_at | image:193081 | radixin | RDX | hs.263671 | nm_002906 | al137751 |
| 202917_s_at | image:1089513 | S100 calcium binding protein | S100A8 | hs.416073 | nm_002964 | nm_002964 |
| | | A8_calgranulin A_{—} | | | | |
| 205487_s_at | image:143622 | vestigial like 1_Drosophila_{—} | VGLL1 | hs.496843 | nm_016267 | nm_016267 |
| 221477_s_at | image:324014 | hypothetical protein MGC5618 | MGC5618 | NA | — | bf575213 |
| 201037_at | image:152714 | phosphofructokinase, platelet | PFKP | hs.26010 | nm_002627 | nm_002627 |
| 202603_at | image:261401 | ADAM metallopeptidase | ADAM10 | hs.172028 | nm_001110 | n51370 |
| | | domain 10 | | | | |
| 210785_s_at | image:307255 | chromosome 1 open reading | C1orf38 | hs.10649 | nm_004848 | ab035482 |
| | | frame 38 | | | | |
| 219386_s_at | image:288807 | SLAM family member 8 | SLAMF8 | hs.438683 | nm_020125 | nm_020125 |
| 203988_s_at | image:156966 | fucosyltransferase 8_alpha_1, | FUT8 | hs.118722 | nm_004480, | nm_004480 |
| | | 6_fucosyltransferase_{—} | | | nm_178154, | |
| | | | | | nm_178155, | |
| | | | | | nm_178156, | |
| | | | | | nm_178157 | |
| 202307_s_at | image:51782 | transporter 1, ATP-binding | TAP1 | hs.352018 | nm_000593 | nm_000593 |
| | | cassette, sub-family | | | | |
| | | B_MDR_TAP_{—} | | | | |
| 210465_s_at | image:219829 | small nuclear RNA activating | SNAPC3 | hs.546299 | nm_003084 | u71300 |
| | | complex, polypeptide 3, 50 kDa | | | | |
| 207498_s_at | image:199680 | cytochrome P450, family 2, | CYP2D6 | hs.534311 | nm_000106, | nm_000106 |
| | | subfamily D, polypeptide 6 | | | nm_001025161 | |
| 215370_at | ipso:0000040 | NA | NA | NA | — | au145394 |
| 209212_s_at | image:208991 | Kruppel-like factor 5_intestinal_{—} | KLF5 | hs.508234 | nm_001730 | ab030824 |
| 219336_s_at | image:50892 | activating signal cointegrator 1 | ASCC1 | hs.500007 | nm_015947 | nm_015947 |
| | | complex subunit 1 | | | | |
| 200709_at | image:159521 | FK506 binding protein 1A, | FKBP1A | hs.471933 | nm_000801, | nm_000801 |
| | | 12 kDa | | | nm_054014 | |
| 229659_s_at | image:159410 | Polymeric immunoglobulin | PIGR | hs.497589 | nm_002644 | be501712 |
| | | receptor | | | | |
| 213572_s_at | ipso:0000605 | serpin peptidase inhibitor, clade | SERPINB1 | hs.381167 | nm_030666 | ai554300 |
| | | B_ovalbumin_, member 1 | | | | |
| 203095_at | image:50754 | mitochondrial translational | MTIF2 | hs.149894 | nm_001005369, | nm_002453 |
| | | initiation factor 2 | | | nm_002453 | |
| 240385_at | image:771332 | GATA binding protein 6 | GATA6 | hs.514746 | nm_005257 | bf002339 |
| 243011_at | image:187120 | family with sequence similarity | FAM55C | hs.130195 | nm_145037 | bf317081 |
| | | 55, member C | | | | |
| 207004_at | image:342181 | B-cell CLL_lymphoma 2 | BCL2 | hs.150749 | nm_000633, | nm_000657 |
| | | | | | nm_000657 | |
| 219718_at | image:187119 | hypothetical protein FLJ10986 | FLJ10986 | hs.444301 | nm_018291 | nm_018291 |
| 202122_s_at | image:188005 | mannose-6-phosphate receptor | M6PRBP1 | hs.140452 | nm_005817 | nm_005817 |
| | | binding protein 1 | | | | |
| 211372_s_at | image:137575 | interleukin 1 receptor, type II | IL1R2 | hs.25333 | nm_004633, | u64094 |
| | | | | | nm_173343 | |
| 220529_at | image:154483 | hypothetical protein FLJ11710 | FLJ11710 | NA | — | nm_024846 |
| 207988_s_at | image:162208 | actin related protein 2_3 | ARPC2 | hs.529303 | nm_005731, | nm_005731 |
| | | complex, subunit 2, 34 kDa | | | nm_152862 | |
| 211430_s_at | image:289337 | immunoglobulin heavy | IGH@_ | hs.510635 | — | m87789 |
| | | locus immunoglobulin | IGHG1 | | | |
| | | heavy | IGHG2 | | | |
| | | constant gamma 1_G1m | IGHG3 | | | |
| | | marker_ immunoglobulin | IGHM | | | |
| | | heavy constant gamma 2_G2m | | | | |
| | | marker immunoglobulin heavy | | | | |
| | | constant gamma 3_G3m | | | | |
| | | marker immunoglobulin heavy | | | | |
| | | constant mu | | | | |
| 220616_at | image:156691 | NA | NA | NA | — | nm_006448 |
| 213502_x_at | image:50877 | similar to | LOC91316 | hs.407693 | xm_498877 | aa398569 |
| | | bK246H3.1_immunoglobulin | | | | |
| | | lambda-like | | | | |
| | | polypeptide 1, pre-B-cell | | | | |
| | | specific_{—} | | | | |

TABLE XI
Metagene underEGFR

| Affymetrix ® | | | | Unigene | Reference | |
|---|---|---|---|---|---|---|
| Probe Set | Clone | Gene | symbol | reference | Sequence (refseq) | Genbank |
| 214440_at | image:145894 | N-acetyltransferase 1_arylamine | NAT1 | hs.155956 | nm_000662 | nm_000662 |
| | | N- | | | | |
| | | acetyltransferase_{—} | | | | |
| 232889_at | image:280743 | hypothetical protein | LOC153561 | NA | nm_207331 | au147591 |
| | | LOC153561 | | | | |
| 219414_at | image:50970 | calsyntenin 2 | CLSTN2 | hs.158529 | nm_022131 | nm_022131 |
| 223044_at | image:489218 | solute carrier family 40_iron- | SLC40A1 | hs.529285 | nm_014585 | al136944 |
| | | regulated transporter_, member 1 | | | | |
| 229381_at | image:155072 | chromosome 1 open reading | C1orf64 | hs.29190 | nm_178840 | ai732488 |
| | | frame 64 | | | | |
| 219197_s_at | image:346321 | signal peptide, CUB domain, | SCUBE2 | hs.523468 | nm_020974 | ai424243 |
| | | EGF-like 2 | | | | |
| 225379_at | image:50764 | microtubule-associated protein | MAPT | hs.101174 | nm_005910, | aa199717 |
| | | tau | | | nm_016834, | |
| | | | | | nm_016835, | |
| | | | | | nm_016841 | |
| 219570_at | image:52103 | chromosome 20 open reading | C20orf23 | hs.101774 | nm_024704 | nm_024704 |
| | | frame 23 | | | | |
| 225789_at | image:188414 | centaurin, gamma 3 | CENTG3 | hs.195048 | nm_031946 | be876194 |
| 219438_at | image:166862 | family with sequence similarity | FAM77C | hs.470259 | nm_024522 | nm_024522 |
| | | 77, member C | | | | |
| 204352_at | image:145410 | TNF receptor-associated factor 5 | TRAF5 | hs.523930 | nm_001033910, | nm_004619 |
| | | | | | nm_004619, | |
| | | | | | nm_145759 | |
| 228994_at | image:52118 | coiled-coil domain containing | CCDC24 | hs.488337 | nm_152499 | au153816 |
| | | 24 | | | | |
| 204550_x_at | image:73778 | glutathione S-transferase M1 | GSTM1 | hs.301961 | nm_000561, | nm_000561 |
| | | | | | nm_146421 | |
| 204623_at | image:298417 | trefoil factor 3_intestinal_{—} | TFF3 | hs.82961 | nm_003226 | nm_003226 |
| 222005_s_at | image:166254 | guanine nucleotide binding | GNG3 | hs.179915 | nm_012202 | al538966 |
| | | protein_G protein_, gamma 3 | | | | |
| 220192_x_at | image:1188588 | SAM pointed domain containing | SPDEF | hs.485158 | nm_012391 | nm_012391 |
| | | ets transcription factor | | | | |
| 218064_s_at | image:171679 | A kinase_PRKA_anchor | AKAP8L | hs.399800 | nm_014371 | nm_014371 |
| | | protein 8-like | | | | |
| 40093_at | image:160656 | Lutheran blood group_Auberger | LU | hs.155048 | nm_001013257, | x83425 |
| | | b antigen included_{—} | | | nm_005581 | |
| 203428_s_at | image:146634 | ASF1 anti-silencing function 1 | ASF1A | hs.292316 | nm_014034 | ab028628 |
| | | homolog A_S. cerevisiae_{—} | | | | |
| 204129_at | image:1756392 | B-cell CLL_lymphoma 9 | BCL9 | hs.415209 | nm_004326 | nm_004326 |
| 224182_x_at | image:166010 | sema domain, transmembrane | SEMA6B | hs.465642 | nm_020241, | af293363 |
| | | domain_TM_, and cytoplasmic | | | nm_032108, | |
| | | domain,_semaphorin_6B | | | nm_133327 | |
| 204418_x_at | image:166910 | glutathione S-transferase M2_muscle_{—} | GSTM2 | hs.279837 | nm_000848 | nm_000848 |
| 201681_s_at | image:153368 | discs, large homolog 5_Drosophila_{—} | DLG5 | hs.500245 | nm_004747 | ab011155 |
| 233955_x_at | image:173797 | CXXC finger 5 | CXXC5 | hs.189119 | nm_016463 | ak001782 |
| 205225_at | image:725321 | estrogen receptor 1 | ESR1 | hs.208124 | nm_000125 | nm_000125 |
| 205201_at | image:767495 | GLI-Kruppel family member | GLI3 | hs.199338 | nm_000168 | nm_000168 |
| | | GLI3_Greig | | | | |
| | | cephalopolysyndactyly | | | | |
| | | syndrome_{—} | | | | |
| 209049_s_at | image:511899 | protein kinase C binding protein 1 | PRKCBP1 | hs.446240 | nm_012408, | bc001004 |
| | | | | | nm_183047, | |
| | | | | | nm_183048 | |
| 218367_x_at | image:183062 | ubiquitin specific peptidase 21 | USP21 | hs.8015 | nm_001014443, | nm_012475 |
| | | | | | nm_012475 | |
| 212099_at | image:768370 | ras homolog gene family, | RHOB | hs.502876 | nm_004040 | ai263909 |
| | | member B | | | | |
| 201613_s_at | image:161763 | adaptor-related protein complex | AP1G2 | hs.343244 | nm_003917, | bc000519 |
| | | 1, gamma 2 subunit | | | nm_080545 | |
| 201754_at | image:278531 | cytochrome c oxidase subunit | COX6C | hs.351875 | nm_004374 | nm_004374 |
| | | VIc | | | | |
| 222282_at | image:155064 | Ubiquitin specific peptidase | USP13 | hs.175322 | nm_003940 | av761453 |
| | | 13_isopeptidase | | | | |
| | | T-3_{—} | | | | |
| 208451_s_at | image:340753 | complement component 4A | C4A C4B | hs.534847 | nm_000592, | nm_000592 |
| | | complement component 4B | | | nm_001002029, | |
| | | complement component 4B, | | | nm_007293 | |
| | | telomeric | | | | |
| 214428_x_at | image:491004 | complement component 4A | C4A C4B | hs.534847 | nm_000592, | k02403 |
| | | complement component 4B | | | nm_001002029, | |
| | | complement component 4B, | | | nm_007293 | |
| | | telomeric | | | | |
| 219426_at | image:155341 | eukaryotic translation initiation | EIF2C3 | hs.567761 | nm_024852, | nm_024852 |
| | | factor 2C, 3 | | | nm_177422 | |
| 209604_s_at | image:139076 | GATA binding protein 3 | GATA3 | hs.524134 | nm_001002295, | bc003070 |
| | | | | | nm_002051 | |
| 201596_x_at | image:151663 | keratin 18 | KRT18 | hs.406013 | nm_000224, | nm_000224 |
| | | | | | nm_199187 | |

TABLE XII
Metagene underER

| Affymetrix ® | | | | | Reference Sequence | |
|---|---|---|---|---|---|---|
| Probe Set | Clone | Gene | symbol | Unigene reference | (refseq) | Genbank |
| 200824_at | image:231424 | glutathione S-transferase pi | GSTP1 | Hs.523836 | NM_000852 | NM_000852 |
| 201037_at | image:152714 | phosphofructokinase, platelet | PFKP | Hs.26010 | NM_002627 | NM_002627 |
| 201201_at | image:51814 | cystatin B (stefin B) | CSTB | Hs.695 | NM_000100 | NM_000100 |
| 201231_s_at | image:392678 | enolase 1, (alpha) | ENO1 | Hs.517145 | NM_001428 | NM_001428 |
| 201487_at | image:320656 | cathepsin C | CTSC | Hs.128065 | NM_001814 | NM_001814 |
| 201579_at | image:1028762 | FAT tumor suppressor homolog | FAT | Hs.481371 | NM_005245 | NM_005245 |
| | | 1 (Drosophila) | | | | |
| 201710_at | image:207378 | v-myb myeloblastosis viral | MYBL2 | Hs.179718 | NM_002466 | NM_002466 |
| | | oncogene homolog (avian)-like 2 | | | | |
| 202705_at | image:845594 | cyclin B2 | CCNB2 | Hs.194698 | NM_004701 | NM_004701 |
| 202967_at | image:345309 | glutathione S-transferase A4 | GSTA4 | Hs.485557 | NM_001512 | NM_001512 |
| 203256_at | ipso:0000143 | cadherin 3, type 1, P-cadherin | CDH3 | Hs.461074 | NM_001793 | NM_001793 |
| | | (placental) | | | | |
| 203287_at | image:121551 | ladinin 1 | LAD1 | Hs.519035 | NM_005558 | NM_005558 |
| 203560_at | image:809588 | gamma-glutamyl hydrolase | GGH | Hs.78619 | NM_003878 | NM_003878 |
| | | (conjugase, | | | | |
| | | folylpolygammaglutamyl | | | | |
| | | hydrolase) | | | | |
| 204092_s_at | image:1912132 | aurora kinase A | AURKA | Hs.250822 | NM_003600 | NM_003600 |
| 204259_at | image:471134 | matrix metallopeptidase 7 | MMP7 | Hs.2256 | NM_002423 | NM_002423 |
| | | (matrilysin, uterine) | | | | |
| 204733_at | image:724109 | kallikrein-related peptidase 6 | KLK6 | Hs.79361 | NM_001012964 | NM_002774 |
| 208370_s_at | ipso:0000077 | regulator of calcineurin 1 | RCAN1 | Hs.282326 | NM_004414 | NM_004414 |
| 208456_s_at | image:278490 | related RAS viral (r-ras) | RRAS2 | Hs.502004 | NM_012250 | NM_012250 |
| | | oncogene homolog 2 | | | | |
| 209791_at | ipso:0000610 | peptidyl arginine deiminase, | PADI2 | Hs.33455 | NM_007365 | AL049569 |
| | | type II | | | | |
| 210453_x_at | ipso:0000267 | ATP synthase, H+ transporting, | ATP5L | Hs.486360 | NM_006476 | AL050277 |
| | | mitochondrial F0 complex, | | | | |
| | | subunit G | | | | |
| 212398_at | image:193081 | radixin | RDX | Hs.263671 | NM_002906 | AI057093 |
| 212501_at | image:161993 | CCAAT/enhancer binding | CEBPB | Hs.517106 | NM_005194 | AL564683 |
| | | protein (C/EBP), beta | | | | |
| 212531_at | image:544683 | lipocalin 2 (oncogene 24p3) | LCN2 | Hs.204238 | NM_005564 | NM_005564 |
| 213094_at | image:259884 | G protein-coupled receptor 126 | GPR126 | Hs.318894 | NM_001032394 | AL033377 |
| 214370_at | image:1089513 | S100 calcium binding protein | S100A8 | Hs.416073 | NM_002964 | AW238654 |
| | | A8 | | | | |
| 215223_s_at | image:324014 | superoxide dismutase 2, | SOD2 | Hs.487046 | NM_000636 | W46388 |
| | | mitochondrial | | | | |
| 215729_s_at | image:143622 | vestigial like 1 (Drosophila) | VGLL1 | Hs.496843 | NM_016267 | BE542323 |
| 217728_at | image:512420 | S100 calcium binding protein | S100A6 | Hs.275243 | NM_014624 | NM_014624 |
| | | A6 | | | | |
| 218060_s_at | image:43457 | chromosome 16 open reading | C16orf57 | Hs.588873 | NM_024598 | NM_024598 |
| | | frame 57 | | | | |
| 221477_s_at | ipso:0000488 | hypothetical protein MGC5618 | MGC5618 | NA | NA | BF575213 |

TABLE XIII
Metagene underPR

| Affymetrix ® | | | | Unigene | Reference Sequence | |
|---|---|---|---|---|---|---|
| Probe Set | Clone | Gene | symbol | reference | (refseq) | Genbank |
| 201487_at | image:320656 | cathepsin C | CTSC | Hs.128065 | NM_001814 | NM_001814 |
| 201505_at | image:428443 | laminin, beta 1 | LAMB1 | Hs.650585 | NM_002291 | NM_002291 |
| 201710_at | image:207378 | v-myb myeloblastosis viral | MYBL2 | Hs.179718 | NM_002466 | NM_002466 |
| | | oncogene homolog (avian)-like 2 | | | | |
| 201710_at | image:724259 | v-myb myeloblastosis viral | MYBL2 | Hs.179718 | NM_002466 | NM_002466 |
| | | oncogene homolog (avian)-like 2 | | | | |
| 202036_s_at | image:783700 | secreted frizzled-related protein 1 | SFRP1 | Hs.213424 | NM_003012 | AF017987 |
| 202246_s_at | image:725349 | cyclin-dependent kinase 4 | CDK4 | Hs.95577 | NM_000075 | NM_000075 |
| 202307_s_at | image:51782 | transporter 1, ATP-binding | TAP1 | Hs.352018 | NM_000593 | NM_000593 |
| | | cassette, sub-family B | | | | |
| | | (MDR/TAP) | | | | |
| 202519_at | image:159783 | MLX interacting protein | MLXIP | Hs.437153 | NM_014938 | NM_014938 |
| 203095_at | image:50754 | mitochondrial translational | MTIF2 | Hs.149894 | NM_001005369 | NM_002453 |
| | | initiation factor 2 | | | | |
| 203256_at | ipso:0000143 | cadherin 3, type 1, P-cadherin | CDH3 | Hs.461074 | NM_001793 | NM_001793 |
| | | (placental) | | | | |
| 203287_at | image:121551 | ladinin 1 | LAD1 | Hs.519035 | NM_005558 | NM_005558 |
| 203685_at | image:342181 | B-cell CLL/lymphoma 2 | BCL2 | Hs.150749 | NM_000633 | NM_000633 |
| 203934_at | image:193857 | kinase insert domain receptor | KDR | Hs.479756 | NM_002253 | NM_002253 |
| | | (a type III receptor tyrosine | | | | |
| | | kinase) | | | | |
| 204470_at | image:323238 | chemokine (C—X—C motif) ligand | CXCL1 | Hs.789 | NM_001511 | NM_001511 |
| | | 1 (melanoma growth stimulating | | | | |
| | | activity, alpha) | | | | |
| 204628_s_at | image:200209 | integrin, beta 3 (platelet | ITGB3 | Hs.218040 | NM_000212 | NM_000212 |
| | | glycoprotein IIIa, antigen CD61) | | | | |
| 205890_s_at | ipso:0000252 | ubiquitin D | UBD | Hs.44532 | NM_006398 | NM_006398 |
| 206324_s_at | image:156808 | death-associated protein kinase 2 | DAPK2 | Hs.237886 | NM_014326 | NM_014326 |
| 206792_x_at | image:219829 | phosphodiesterase 4C, cAMP- | PDE4C | Hs.631628 | NM_000923 | NM_000923 |
| | | specific (phosphodiesterase E1 | | | | |
| | | dunce homolog, Drosophila) | | | | |
| 207270_x_at | image:156937 | CD300c molecule | CD300C | Hs.2605 | NM_006678 | NM_006678 |
| 207498_s_at | image:199680 | cytochrome P450, family 2, | CYP2D6 | Hs.648256 | NM_000106 | NM_000106 |
| | | subfamily D, polypeptide 6 | | | | |
| 207571_x_at | image:307255 | chromosome 1 open reading | C1orf38 | Hs.10649 | NM_001039477 | NM_004848 |
| | | frame 38 | | | | |
| 209138_x_at | ipso:0000434 | immunoglobulin lambda locus | IGL@ | Hs.449585 | NA | M87790 |
| 209791_at | ipso:0000610 | peptidyl arginine deiminase, | PADI2 | Hs.33455 | NM_007365 | AL049569 |
| | | type II | | | | |
| 209848_s_at | image:342383 | silver homolog (mouse) | SILV | Hs.95972 | NM_006928 | U01874 |
| 210002_at | image:771332 | GATA binding protein 6 | GATA6 | Hs.514746 | NM_005257 | D87811 |
| 211372_s_at | image:137575 | interleukin 1 receptor, type II | IL1R2 | Hs.25333 | NM_004633 | U64094 |
| 211430_s_at | image:289337 | immunoglobulin heavy constant | IGHG3 | Hs.510635 | NA | M87789 |
| | | gamma 3 (G3m marker) | | | | |
| 212398_at | image:193081 | radixin | RDX | Hs.263671 | NM_002906 | AI057093 |
| 212531_at | image:544683 | lipocalin 2 (oncogene 24p3) | LCN2 | Hs.204238 | NM_005564 | NM_005564 |
| 213572_s_at | ipso:0000605 | serpin peptidase inhibitor, clade | SERPINB1 | Hs.381167 | NM_030666 | AI554300 |
| | | B (ovalbumin), member 1 | | | | |
| 214370_at | image:1089513 | S100 calcium binding protein | S100A8 | Hs.416073 | NM_002964 | AW238654 |
| | | A8 | | | | |
| 215223_s_at | image:324014 | superoxide dismutase 2, | SOD2 | Hs.487046 | NM_000636 | W46388 |
| | | mitochondrial | | | | |
| 215729_s_at | image:143622 | vestigial like 1 (Drosophila) | VGLL1 | Hs.496843 | NM_016267 | BE542323 |
| 215946_x_at | image:50877 | similar to omega protein | CTA-246H3.1 | Hs.567636 | NM_001013618 | AL022324 |
| 216598_s_at | ipso:0000152 | chemokine (C-C motif) ligand 2 | CCL2 | Hs.303649 | NM_002982 | S69738 |
| 217865_at | image:186926 | ring finger protein 130 | RNF130 | Hs.484363 | NM_018434 | NM_018434 |
| 219386_s_at | image:288807 | SLAM family member 8 | SLAMF8 | Hs.438683 | NM_020125 | NM_020125 |
| 221651_x_at | image:156691 | immunoglobulin kappa constant | IGKC | Hs.449621 | NA | BC005332 |
| 221671_x_at | ipso:0000376 | immunoglobulin kappa constant | IGKC | Hs.449621 | NA | M63438 |
| 224795_x_at | image:713852 | immunoglobulin kappa constant | IGKC | Hs.449621 | NA | AW575927 |
| 227262_at | image:187120 | hyaluronan and proteoglycan | HAPLN3 | Hs.447530 | NM_178232 | BE348293 |
| | | link protein 3 | | | | |
| 243209_at | image:156966 | potassium voltage-gated | KCNQ4 | Hs.473058 | NM_004700 | BF725804 |
| | | channel, KQT-like subfamily, | | | | |
| | | member 4 | | | | |

TABLE XIV
Metagene underEGFR

| Affymetrix ® | | | | Unigene | Reference Sequence | |
|---|---|---|---|---|---|---|
| Probe Set | Clone | Gene | symbol | reference | (refseq) | Genbank |
| 200670_at | ipso:0000125 | X-box binding protein 1 | XBP1 | Hs.437638 | NM_005080 | NM_001079539 |
| 200670_at | image:301950 | X-box binding protein 1 | XBP1 | Hs.437638 | NM_005080 | NM_001079539 |
| 201596_x_at | image:151663 | keratin 18 | KRT18 | Hs.406013 | NM_000224 | NM_000224 |
| 201613_s_at | image:161763 | adaptor-related protein complex | AP1G2 | Hs.343244 | BC000519 | NM_003917 |
| | | 1, gamma 2 subunit | | | | |
| 201681_s_at | image:153368 | discs, large homolog 5 | DLG5 | Hs.654780 | AB011155 | NM_004747 |
| | | (Drosophila) | | | | |
| 201754_at | image:278531 | cytochrome c oxidase subunit | COX6C | Hs.351875 | NM_004374 | NM_004374 |
| | | VIc | | | | |
| 201860_s_at | image:160149 | plasminogen activator, tissue | PLAT | Hs.491582 | NM_000930 | NM_000930 |
| 201860_s_at | ipso:0000253 | plasminogen activator, tissue | PLAT | Hs.491582 | NM_000930 | NM_000930 |
| 204129_at | image:1756392 | B-cell CLL/lymphoma 9 | BCL9 | Hs.415209 | NM_004326 | NM_004326 |
| 204352_at | image:145410 | TNF receptor-associated factor 5 | TRAF5 | Hs.523930 | NM_004619 | NM_001033910 |
| 204418_x_at | image:166910 | glutathione S-transferase M2 | GSTM2 | Hs.279837 | NM_000848 | NM_000848 |
| | | (muscle) | | | | |
| 204418_x_at | image:153444 | glutathione S-transferase M2 | GSTM2 | Hs.279837 | NM_000848 | NM_000848 |
| | | (muscle) | | | | |
| 204418_x_at | image:664233 | glutathione S-transferase M2 | GSTM2 | Hs.279837 | NM_000848 | NM_000848 |
| | | (muscle) | | | | |
| 204550_x_at | image:73778 | glutathione S-transferase M1 | GSTM1 | Hs.301961 | NM_000561 | NM_000561 |
| 204623_at | image:298417 | trefoil factor 3 (intestinal) | TFF3 | Hs.82961 | NM_003226 | NM_003226 |
| 205009_at | image:1075949 | trefoil factor 1 | TFF1 | Hs.162807 | NM_003225 | NM_003225 |
| 205186_at | image:782688 | dynein, axonemal, light | DNALI1 | Hs.406050 | NM_003462 | NM_003462 |
| | | intermediate chain 1 | | | | |
| 205201_at | image:767495 | GLI-Kruppel family member | GLI3 | Hs.21509 | NM_000168 | NM_000168 |
| | | GLI3 (Greig | | | | |
| | | cephalopolysyndactyly | | | | |
| | | syndrome) | | | | |
| 205225_at | image:725321 | estrogen receptor 1 | ESR1 | Hs.208124 | NM_000125 | NM_000125 |
| 206107_at | image:277917 | regulator of G-protein signaling | RGS11 | Hs.65756 | NM_003834 | NM_003834 |
| | | 11 | | | | |
| 206289_at | image:785930 | homeobox A4 | HOXA4 | Hs.654466 | NM_002141 | NM_002141 |
| 206401_s_at | image:50764 | microtubule-associated protein | MAPT | Hs.101174 | J03778 | NM_005910 |
| | | tau | | | | |
| 208451_s_at | image:340753 | complement component 4A | C4A | Hs.655564 | NM_000592 | NM_007293 |
| | | (Rodgers blood group) | | | | |
| 208451_s_at | image:491004 | complement component 4A | C4A | Hs.655564 | NM_000592 | NM_007293 |
| | | (Rodgers blood group) | | | | |
| 209048_s_at | image:511899 | zinc finger, MYND-type | ZMYND8 | Hs.446240 | AB032951 | NM_012408 |
| | | containing 8 | | | | |
| 209604_s_at | image:139076 | GATA binding protein 3 | GATA3 | Hs.524134 | BC003070 | NM_001002295 |
| 209604_s_at | ipso:0000286 | GATA binding protein 3 | GATA3 | Hs.524134 | BC003070 | NM_001002295 |
| 210108_at | image:49630 | calcium channel, voltage- | CACNA1D | Hs.476358 | BE550599 | NM_000720 |
| | | dependent, L type, alpha 1D | | | | |
| | | subunit | | | | |
| 210272_at | image:182295 | cytochrome P450, family 2, | CYP2B7P1 | Hs.529117 | M29873 | NR_001278 |
| | | subfamily B, polypeptide 7 | | | | |
| | | pseudogene 1 | | | | |
| 211038_s_at | image:149567 | ciliary rootlet coiled-coil, | CROCCL1 | Hs.631865 | BC006312 | XM_001130627 |
| | | rootletin-like 1 | | | | |
| 212099_at | image:768370 | ras homolog gene family, | RHOB | Hs.502876 | AI263909 | NM_004040 |
| | | member B | | | | |
| 212099_at | image:149760 | ras homolog gene family, | RHOB | Hs.502876 | AI263909 | NM_004040 |
| | | member B | | | | |
| 214440_at | image:145894 | N-acetyltransferase 1 | NAT1 | Hs.591847 | NM_000662 | NM_000662 |
| | | (arylamine N-acetyltransferase) | | | | |
| 218064_s_at | image:171679 | A kinase (PRKA) anchor protein | AKAP8L | Hs.399800 | NM_014371 | NM_014371 |
| | | 8-like | | | | |
| 218211_s_at | image:155341 | melanophilin | MLPH | Hs.102406 | NM_024101 | NM_001042467 |
| 218692_at | image:149549 | Golgi-localized protein | GOLSYN | Hs.390738 | NM_017786 | NM_001099743 |
| 219197_s_at | image:346321 | signal peptide, CUB domain, | SCUBE2 | Hs.523468 | AI424243 | NM_020974 |
| | | EGF-like 2 | | | | |
| 219438_at | image:166862 | family with sequence similarity | FAM77C | Hs.470259 | NM_024522 | NM_024522 |
| | | 77, member C | | | | |
| 219570_at | image:52103 | chromosome 20 open reading | C20orf23 | Hs.101774 | NM_024704 | NM_024704 |
| | | frame 23 | | | | |
| 220192_x_at | image:1188588 | SAM pointed domain containing | SPDEF | Hs.485158 | NM_012391 | NM_012391 |
| | | ets transcription factor | | | | |
| 220778_x_at | image:52741 | sema domain, transmembrane | SEMA6B | Hs.465642 | NM_020241 | NM_020241 |
| | | domain (TM), and cytoplasmic | | | | |
| | | domain, (semaphorin) 6B | | | | |
| 222005_s_at | image:166254 | guanine nucleotide binding | GNG3 | Hs.179915 | AL538966 | NM_012202 |
| | | protein (G protein), gamma 3 | | | | |
| 223044_at | image:489218 | solute carrier family 40 (iron- | SLC40A1 | Hs.643005 | AL136944 | NM_014585 |
| | | regulated transporter), member 1 | | | | |
| 223721_s_at | image:120138 | DnaJ (Hsp40) homolog, | DNAJC12 | Hs.260720 | AF176013 | NM_021800 |
| | | subfamily C, member 12 | | | | |
| 224516_s_at | image:173797 | CXXC finger 5 | CXXC5 | Hs.189119 | BC006428 | NM_016463 |
| 225092_at | image:772890 | nucleoporin 88 kDa | NUP88 | Hs.584784 | AL550977 | NM_002532 |
| 225883_at | image:50602 | ATG16 autophagy related 16- | ATG16L2 | Hs.653186 | AK024423 | NM_033388 |
| | | like 2 (S. cerevisiae) | | | | |
| 225911_at | image:266500 | nephronectin | NPNT | Hs.518921 | AL138410 | NM_001033047 |
| 226362_at | image:280743 | small EDRK-rich factor 1A | SERF1A | Hs.658079 | AI198515 | NM_021967 |
| | | (telomeric) | | | | |
| 226373_at | image:147138 | sideroflexin 5 | SFXN5 | Hs.368171 | AW166098 | NM_144579 |
| 226506_at | image:160656 | thrombospondin, type I, domain | THSD4 | Hs.387057 | AI742570 | NM_024817 |
| | | containing 4 | | | | |
| 227425_at | image:43488 | RALBP1 associated Eps | REPS2 | Hs.186810 | AI984607 | NM_001080975 |
| | | domain containing 2 | | | | |
| 227515_at | image:188414 | STAM binding protein | STAMBP | Hs.469018 | AU158421 | NM_006463 |
| 227550_at | image:44338 | hypothetical protein | LOC143381 | Hs.388347 | AW242720 | NA |
| | | LOC143381 | | | | |
| 227811_at | image:146634 | FYVE, RhoGEF and PH | FGD3 | Hs.411081 | AK000004 | NM_001083536 |
| | | domain containing 3 | | | | |
| 228528_at | image:153617 | NA | NA | NA | AI927692 | NA |
| 228994_at | image:52118 | coiled-coil domain containing | CCDC24 | Hs.632394 | AU153816 | NM_152499 |
| | | 24 | | | | |
| 229150_at | ipso:0000614 | melanophilin | MLPH | Hs.102406 | AI810764 | NM_001042467 |
| 229381_at | image:155072 | chromosome 1 open reading | C1orf64 | Hs.29190 | AI732488 | NM_178840 |
| | | frame 64 | | | | |

The above described protocol for finding a correspondence between a cDNA platform (e.g., Discovery™) and another platform (e.g., Affymetrix®) may be similarly applied by a person skilled in the art for the other metagenes according to the present invention.

1-89. (canceled)

90. A method of assessing the clinical outcome of a female mammal suffering from breast cancer, comprising the steps of:
a) generating a metagene adjusted value underER by comparing the expression level, in a biological sample from said female mammal and in a control, of at least 10 nucleic acid sequences selected in the group comprising or consisting of: SEQ ID No:374 (nm_{—}000212), SEQ ID No:1027 (nm_{—}007365), SEQ ID No:598 (nm_{—}000636), SEQ ID No:717 (nm_{—}024598), SEQ ID No:573 (nm_{—}001527), SEQ ID No:83 (nm_{—}015065), SEQ ID No:12 (nm_{—}002964), SEQ ID No:405 (nm_{—}000852), SEQ ID No:856 (nm_{—}005564), SEQ ID No:384 (nm_{—}002466), SEQ ID No:167 (nm_{—}002627), SEQ ID No:51 (nm_{—}198433), SEQ ID No:999 (nm_{—}145290), SEQ ID No:979 (nm_{—}004414), SEQ ID No:2 (nm_{—}005245), SEQ ID No:98 (nm_{—}016267), SEQ ID No:751 (nm_{—}002423), SEQ ID No:696 (nm_{—}001428), SEQ ID No:1050 (BC034638), SEQ ID No:488 (nm_{—}002979), SEQ ID No:262 (nm_{—}005194), SEQ ID No:1020 (nm_{—}000359), SEQ ID No:1106 (BC015969), SEQ ID No:952 (nm_{—}003878), SEQ ID No:675 (nm_{—}001512), SEQ ID No:289 (nm_{—}020179), SEQ ID No:553 (nm_{—}004701), SEQ ID No:579 (nm_{—}001814), SEQ ID No:760 (nm_{—}005746), SEQ ID No:805 (nm_{—}014624), SEQ ID No:361 (nm_{—}002906), SEQ ID No:448 (nm_{—}198569), SEQ ID No:170 (nm_{—}002428), SEQ ID No:878 (nm_{—}002774), SEQ ID No:1117, SEQ ID No:612 (nm_{—}032515), SEQ ID No:540 (nm_{—}003159), SEQ ID No:823 (nm_{—}000100), SEQ ID No:131 (nm_{—}145280), SEQ ID No:705 (nm_{—}005596), SEQ ID No:31 (nm_{—}005558), and SEQ ID No:199 (nm_{—}024323) fragments, derivatives or complementary sequences thereof;
b) generating a metagene adjusted value underPR by comparing the expression level, in a biological sample from said female mammal and in a control, of at least 6 nucleic acid sequences selected in the group comprising or consisting of: SEQ ID No:598 (nm_{—}000636), SEQ ID No:1122, SEQ ID No:364 (nm_{—}002253), SEQ ID No:387 (nm_{—}006563), SEQ ID No:34 (nm_{—}001229), SEQ ID No:657 (nm_{—}000633), SEQ ID No:384 (nm_{—}002466), SEQ ID No:451 (nm_{—}001110), SEQ ID No:999 (nm_{—}145290), SEQ ID No:1056 (AK126297), SEQ ID No:15 (nm_{—}003243), SEQ ID No:1090 (AK125808), SEQ ID No:1120, SEQ ID No:12 (nm_{—}002964), SEQ ID No:743 (nm_{—}006875), SEQ ID No:414 (nm_{—}000546), SEQ ID No:374 (nm_{—}000212), SEQ ID No:711 (nm_{—}002291), SEQ ID No:663 (nm_{—}006928), SEQ ID No:1102 (AK124587), SEQ ID No:237 (nm_{—}002644), SEQ ID No:60 (nm_{—}022640), SEQ ID No:361 (nm_{—}002906), SEQ ID No:119 (nm_{—}004730) (or SEQ ID No:1109 (NM_{—}002019)), SEQ ID No:167 (nm_{—}002627), SEQ ID No:339 (nm_{—}144970), SEQ ID No:333 (nm_{—}145037), SEQ ID No:83 (nm_{—}015065), SEQ ID No:330 (nm_{—}018291), SEQ ID No:1024 (nm_{—}030666), SEQ ID No:229 (nm_{—}004586), SEQ ID No:925 (nm_{—}005257), SEQ ID No:788 (nm_{—}001005369), SEQ ID No:1104 (AK128524), SEQ ID No:1103 (BX108410), SEQ ID No:66 (nm_{—}000416), SEQ ID No:1030 (nm_{—}024007), SEQ ID No:1119, SEQ ID No:1068 (AK024670), SEQ ID No:241 (nm_{—}000801), SEQ ID No:398 (nm_{—}003084), SEQ ID No:74 (nm_{—}000878), SEQ ID No:1087 (AK074131), SEQ ID No:955 (nm_{—}001986), SEQ ID No:71 (nm_{—}004633), SEQ ID No:1105 (BC072392), SEQ ID No:856 (nm_{—}005564), SEQ ID No:231 (nm_{—}006678), SEQ ID No:593 (nm_{—}001511), SEQ ID No:384 (nm_{—}002466), SEQ ID No:519 (nm_{—}020125), SEQ ID No:579 (nm_{—}001814), SEQ ID No:1039 (nm_{—}006209), SEQ ID No:31 (nm_{—}005558), SEQ ID No:327 (nm_{—}173825), SEQ ID No:573 (nm_{—}001527), SEQ ID No:98 (nm_{—}016267), SEQ ID No:1059 (AK091113), SEQ ID No:886 (nm_{—}000075), SEQ ID No:1032 (nm_{—}005688), SEQ ID No:1091 (XM_{—}378178), SEQ ID No:233 (nm_{—}178155), SEQ ID No:938 (nm_{—}003012), SEQ ID No:264 (nm_{—}152862), SEQ ID No:546 (nm_{—}005874), SEQ ID No:1099 (BC066343) SEQ ID No:1037 (nm_{—}023068), SEQ ID No:550 (nm_{—}004848), SEQ ID No:1027 (nm_{—}007365), SEQ ID No:1005 (nm_{—}014938), SEQ ID No:820 (nm_{—}000593), and SEQ ID No:370 (nm_{—}000106), fragments, derivatives or complementary sequences thereof;
c) generating a metagene adjusted value underEGFR by comparing the level, in a biological sample from said female mammal and in a control, of at least 10 nucleic acid sequences selected in the group comprising or consisting of: SEQ ID No:1071 (NM_{—}001033047), SEQ ID No:254 (nm_{—}005581), SEQ ID No:6 (nm_{—}003225), SEQ ID No:883 (nm_{—}000125), SEQ ID No:543 (nm_{—}005080), SEQ ID No:681 (nm_{—}020974), SEQ ID No:63 (nm_{—}001002295), SEQ ID No:212 (nm_{—}024852), SEQ ID No:635 (nm_{—}001002029), SEQ ID No:535 (nm_{—}003226), SEQ ID No:1125, SEQ ID No:109 (nm_{—}000662), SEQ ID No:342 (nm_{—}001846), SEQ ID No:927 (nm_{—}004703), SEQ ID No:1124, SEQ ID No:124 (nm_{—}014899), SEQ ID No:280 (nm_{—}020764) (or SEQ ID No:1110 (NM_{—}024522)), SEQ ID No:297 (nm_{—}016463), SEQ ID No:791 (nm_{—}016835), SEQ ID No:210 (nm_{—}178840), SEQ ID No:827 (nm_{—}152499), SEQ ID No:1064 (NM_{—}000767), SEQ ID No:147 (nm_{—}014675), SEQ ID No:323 (nm_{—}001014443), SEQ ID No:106 (nm_{—}004619), SEQ ID No:181 (nm_{—}000848), SEQ ID No:376 (nm_{—}057158), SEQ ID No:116 (nm_{—}014034), SEQ ID No:252 (nm_{—}000758), SEQ ID No:797 (nm_{—}022131), SEQ ID No:911 (nm_{—}000168), SEQ ID No:720 (nm_{—}004726), SEQ ID No:889 (nm_{—}000561), SEQ ID No:250 (nm_{—}000930), SEQ ID No:179 (nm_{—}004747), SEQ ID No:786 (nm_{—}033388), SEQ ID No:177 (nm_{—}015996), SEQ ID No:1047 (BC012900), SEQ ID No:301 (nm_{—}004326), SEQ ID No:207 (nm_{—}003940), SEQ ID No:936 (nm_{—}003462), SEQ ID No:916 (nm_{—}001453) (or SEQ ID No:1116 (NM_{—}004040)), SEQ ID No:1052 (BX096026), SEQ ID No:159 (nm_{—}000224), SEQ ID No:1096 (AK127274), SEQ ID No:28 (nm_{—}021800), SEQ ID No:1054 (AK123264), SEQ ID No:25 (nm_{—}012391) (or SEQ ID No:1108 (NM_{—}053279)), SEQ ID No:825 (nm_{—}024704), SEQ ID No:145 (nm_{—}017786), SEQ ID No:491 (nm_{—}004374), SEQ ID No:485 (nm_{—}003834), SEQ ID No:1072 (AY007114), SEQ ID No:274 (nm_{—}032108), SEQ ID No:258 (nm_{—}080545), SEQ ID No:292 (nm_{—}014371), SEQ ID No:803 (nm_{—}183047), SEQ ID No:349 (nm_{—}031946), SEQ ID No:1123, SEQ ID No:763 (nm_{—}014585), SEQ ID No:438 (nm_{—}001759), SEQ ID No:94 (nm_{—}014315), SEQ ID No:845 (nm_{—}001089), SEQ ID No:1084 (BX648964), SEQ ID No:734 (nm_{—}025137), SEQ ID No:943 (nm_{—}002141), SEQ ID No:1085 (NM_{—}000720), and SEQ ID No:276 (nm_{—}012202), fragments, derivatives or complementary sequences thereof;
d) generating a score (S_{C}) from said metagene adjusted values using a mathematical method establishing a relation between the combined metagene values and the clinical outcome of said female mammal.

91. The method of claim 90, wherein said metagene adjusted value underER is generated by comparing the expression level, in a biological sample from said female mammal and in a control, of the 20 nucleic acid sequences selected in the group consisting of: SEQ ID No:374 (nm_{—}000212); SEQ ID No:1027 (nm_{—}007365); SEQ ID No:598 (nm_{—}000636); SEQ ID No:573 (nm_{—}001527); SEQ ID No:83 (nm_{—}015065); SEQ ID No:12 (nm_{—}002964); SEQ ID No:405 (nm_{—}000852); SEQ ID No:856 (nm_{—}005564); SEQ ID No:167 (nm_{—}002627); SEQ ID No:51 (nm_{—}198433); SEQ ID No:98 (nm_{—}016267); SEQ ID No:751 (nm_{—}002423); SEQ ID No:696 (nm_{—}001428); SEQ ID No:262 (nm_{—}005194); SEQ ID No:1020 (nm_{—}000359); SEQ ID No:579 (nm_{—}001814); SEQ ID No:760 (nm_{—}005746); SEQ ID No:805 (nm_{—}014624); SEQ ID No:878 (nm_{—}002774); and SEQ ID No:612 (nm_{—}032515), fragments, derivatives or complementary sequences thereof.

92. The method of claim 90, wherein said metagene adjusted value underER is generated by comparing the expression level, in a biological sample from said female mammal and in a control, of the 27 nucleic acid sequences selected in the group consisting of: SEQ ID No:374 (nm_{—}000212); SEQ ID No:1027 (nm_{—}007365); SEQ ID No:598 (nm_{—}000636); SEQ ID No:573 (nm_{—}001527); SEQ ID No:83 (nm_{—}015065); SEQ ID No:12 (nm_{—}002964); SEQ ID No:405 (nm_{—}000852); SEQ ID No:856 (nm_{—}005564); SEQ ID No:167 (nm_{—}002627); SEQ ID No:51 (nm_{—}198433); SEQ ID No:98 (nm_{—}016267); SEQ ID No:751 (nm_{—}002423); SEQ ID No:696 (nm_{—}001428); SEQ ID No:262 (nm_{—}005194); SEQ ID No:1020 (nm_{—}000359); SEQ ID No:579 (nm_{—}001814); SEQ ID No:760 (nm_{—}005746); SEQ ID No:805 (nm_{—}014624); SEQ ID No:878 (nm_{—}002774); SEQ ID No:612 (nm_{—}032515); SEQ ID No:384 (nm_{—}002466); SEQ ID No:2 (nm_{—}005245); SEQ ID No:1050 (BC034638); SEQ ID No:952 (nm_{—}003878); SEQ ID No:361 (nm_{—}002906); SEQ ID No:31 (nm_{—}005558); and SEQ ID No:199 (nm_{—}024323), fragments, derivatives or complementary sequences thereof.

93. The method of claim 90, wherein said metagene adjusted value underPR is generated by comparing the expression level, in a biological sample from said female mammal and in a control, of the 6 nucleic acid sequences selected in the group consisting of: SEQ ID No:364 (nm_{—}002253); SEQ ID No:34 (nm_{—}001229); SEQ ID No:657 (nm_{—}000633); SEQ ID No:339 (nm_{—}144970); SEQ ID No:229 (nm_{—}004586); SEQ ID No:1119, fragments, derivatives or complementary sequences thereof.

94. The method of claim 90, wherein said metagene adjusted value underPR is generated by comparing the expression level, in a biological sample from said female mammal and in a control, of the 36 nucleic acid sequences selected in the group consisting of: SEQ ID No:364 (nm_{—}002253); SEQ ID No:34 (nm_{—}001229); SEQ ID No:657 (nm_{—}000633); SEQ ID No:339 (nm_{—}144970); SEQ ID No:229 (nm_{—}004586); SEQ ID No:1119; SEQ ID No:387 (nm_{—}006563); SEQ ID No:1056 (AK126297); SEQ ID No:15 (nm_{—}003243); SEQ ID No:1120; SEQ ID No:414 (nm_{—}000546); SEQ ID No:374 (nm_{—}000212); SEQ ID No:711 (nm_{—}002291); SEQ ID No:663 (nm_{—}006928); SEQ ID No:237 (nm_{—}002644); SEQ ID No:60 (nm_{—}022640); SEQ ID No:119 (nm_{—}004730); SEQ ID No:330 (nm_{—}018291); SEQ ID No:1024 (nm_{—}030666); SEQ ID No:925 (nm_{—}005257); SEQ ID No:1104 (AK128524); SEQ ID No:1103 (BX108410); SEQ ID No:66 (nm_{—}000416); SEQ ID No:1068 (AK024670); SEQ ID No:374 (nm_{—}000212); SEQ ID No:74 (nm_{—}000878); SEQ ID No:231 (nm_{—}006678); SEQ ID No:593 (nm_{—}001511); SEQ ID No:384 (nm_{—}002466); SEQ ID No:1039 (nm_{—}006209); SEQ ID No:327 (nm_{—}173825); SEQ ID No:886 (nm_{—}000075); SEQ ID No:1032 (nm_{—}005688); SEQ ID No:264 (nm_{—}152862); SEQ ID No:1037 (nm_{—}023068); and SEQ ID No:1005 (nm_{—}014938), fragments, derivatives or complementary sequences thereof.

95. The method of claim 90, wherein said metagene adjusted value underEGFR is generated by comparing the expression level, in a biological sample from said female mammal and in a control, of the 24 nucleic acid sequences selected in the group consisting of: SEQ ID No:1071 (nm_{—}001033047); SEQ ID No:254 (nm_{—}005581); SEQ ID No:6 (nm_{—}003225); SEQ ID No:883 (nm_{—}000125); SEQ ID No:543 (nm_{—}005080); SEQ ID No:681 (nm_{—}020974); SEQ ID No:63 (nm_{—}001002295); SEQ ID No:212 (nm_{—}024852); SEQ ID No:635 (nm_{—}001002029); SEQ ID No:535 (nm_{—}003226); SEQ ID No:1125); SEQ ID No:1124; SEQ ID No:297 (nm_{—}016463); SEQ ID No:791 (nm_{—}016835); SEQ ID No:827 (nm_{—}152499); SEQ ID No:207 (nm_{—}003940); SEQ ID No:916 (nm_{—}001453) (or SEQ ID No:1116 (nm_{—}004040)); SEQ ID No:1052 (BX096026); SEQ ID No:159 (nm_{—}000224); SEQ ID No:25 (nm_{—}012391) (or SEQ ID No:1108 (NM_{—}053279)); SEQ ID No:845 (nm_{—}001089); and SEQ ID No:1085 (NM_{—}000720), fragments, derivatives or complementary sequences thereof.

96. The method of claim 90, wherein said metagene adjusted value underEGFR is generated by comparing the expression level, in a biological sample from said female mammal and in a control, of the 37 nucleic acid sequences selected in the group consisting of: SEQ ID No:1071 (nm_{—}001033047); SEQ ID No:254 (nm_{—}005581); SEQ ID No:6 (nm_{—}003225); SEQ ID No:883 (nm_{—}000125); SEQ ID No:543 (nm_{—}005080); SEQ ID No:681 (nm_{—}020974); SEQ ID No:63 (nm_{—}001002295); SEQ ID No:212 (nm_{—}024852); SEQ ID No:635 (nm_{—}001002029); SEQ ID No:535 (nm_{—}003226); SEQ ID No:1125; SEQ ID No:1124; SEQ ID No:297 (nm_{—}016463); SEQ ID No:791 (nm_{—}016835); SEQ ID No:827 (nm_{—}152499); SEQ ID No:207 (nm_{—}003940); SEQ ID No:916 (nm_{—}001453) (or SEQ ID No:1116 (nm_{—}004040)); SEQ ID No:1052 (BX096026); SEQ ID No:159 (nm_{—}000224); SEQ ID No:25 (nm_{—}012391) (or SEQ ID No:1108 (NM_{—}053279)); SEQ ID No:845 (nm_{—}001089); SEQ ID No:1085 (NM_{—}000720); SEQ ID No:109 (nm_{—}000662); SEQ ID No:342 (nm_{—}001846); SEQ ID No:927 (nm_{—}004703); SEQ ID No:280 (nm_{—}020764) (or SEQ ID No:1110 (NM_{—}024522)); SEQ ID No:210 (nm_{—}178840); SEQ ID No:181 (nm_{—}000848); SEQ ID No:116 (nm_{—}014034); SEQ ID No:250 (nm_{—}000930); SEQ ID No:177 (nm_{—}015996); SEQ ID No:825 (nm_{—}024704); SEQ ID No:145 (nm_{—}017786); and SEQ ID No:276 (nm_{—}012202), fragments, derivatives or complementary sequences thereof.

97. A method of assessing the clinical outcome of a female mammal suffering from breast cancer, comprising the steps of:
a) generating a metagene adjusted value underEGFR by comparing the expression level, in a biological sample from said female mammal and in a control, of at least one nucleic acid sequence selected in the group consisting of: SEQ ID No:1071 (NM_{—}001033047), SEQ ID No:254 (nm_{—}005581), SEQ ID No:6 (nm_{—}003225), SEQ ID No:883 (nm_{—}000125), SEQ ID No:543 (nm_{—}005080), SEQ ID No:681 (nm_{—}020974), SEQ ID No:63 (nm_{—}001002295), SEQ ID No:212 (nm_{—}024852), SEQ ID No:635 (nm_{—}001002029), SEQ ID No:535 (nm_{—}003226), SEQ ID No:1125, SEQ ID No:109 (nm_{—}000662), SEQ ID No:342 (nm_{—}001846), SEQ ID No:927 (nm_{—}004703), SEQ ID No:1124, SEQ ID No:124 (nm_{—}014899), SEQ ID No:280 (nm_{—}020764) (or SEQ ID No:1110 (NM_{—}024522)), SEQ ID No:297 (nm_{—}016463), SEQ ID No:791 (nm_{—}016835), SEQ ID No:210 (nm_{—}178840), SEQ ID No:827 (nm_{—}152499), SEQ ID No:1064 (NM_{—}000767), SEQ ID No:147 (nm_{—}014675), SEQ ID No:323 (nm_{—}001014443), SEQ ID No:106 (nm_{—}004619), SEQ ID No:181 (nm_{—}000848), SEQ ID No:376 (nm_{—}057158), SEQ ID No:116 (nm_{—}014034), SEQ ID No:252 (nm_{—}000758), SEQ ID No:797 (nm_{—}022131), SEQ ID No:911 (nm_{—}000168), SEQ ID No:720 (nm_{—}004726), SEQ ID No:889 (nm_{—}000561), SEQ ID No:250 (nm_{—}000930), SEQ ID No:179 (nm_{—}004747), SEQ ID No:786 (nm_{—}033388), SEQ ID No:177 (nm_{—}015996), SEQ ID No:1047 (BC012900), SEQ ID No:301 (nm_{—}004326), SEQ ID No:207 (nm_{—}003940), SEQ ID No:936 (nm_{—}003462), SEQ ID No:916 (nm_{—}001453) (or SEQ ID No:1116 (NM_{—}004040)), SEQ ID No:1052 (BX096026), SEQ ID No:159 (nm_{—}000224), SEQ ID No:1096 (AK127274), SEQ ID No:28 (nm_{—}021800), SEQ ID No:1054 (AK123264), SEQ ID No:25 (nm_{—}012391) (or SEQ ID No:1108 (NM_{—}053279)), SEQ ID No:825 (nm_{—}024704), SEQ ID No:145 (nm_{—}017786), SEQ ID No:491 (nm_{—}004374), SEQ ID No:485 (nm_{—}003834), SEQ ID No:1072 (AY007114), SEQ ID No:274 (nm_{—}032108), SEQ ID No:258 (nm_{—}080545), SEQ ID No:292 (nm_{—}014371), SEQ ID No:803 (nm_{—}183047), SEQ ID No:349 (nm_{—}031946), SEQ ID No:1123, SEQ ID No:763 (nm_{—}014585), SEQ ID No:438 (nm_{—}001759), SEQ ID No:94 (nm_{—}014315), SEQ ID No:845 (nm_{—}001089), SEQ ID No:1084 (BX648964), SEQ ID No:734 (nm_{—}025137), SEQ ID No:943 (nm_{—}002141), SEQ ID No:1085 (NM_{—}000720), and SEQ ID No:276 (nm_{—}012202), fragments, derivatives or complementary sequences thereof;
b) generating a metagene adjusted value overEGFR by comparing the expression level, in a biological sample from said female mammal and in a control, of at least one nucleic acid sequences selected in the group consisting of SEQ ID No:405 (nm_{—}000852), SEQ ID No:374 (nm_{—}000212), SEQ ID No:1122, SEQ ID No:598 (nm_{—}000636), SEQ ID No:262 (nm_{—}005194), SEQ ID No:1099 (BC066343), SEQ ID No:696 (nm_{—}001428), SEQ ID No:1059 (AK091113), SEQ ID No:751 (nm_{—}002423), SEQ ID No:1121, SEQ ID No:286 (nm_{—}002417), SEQ ID No:244 (nm_{—}199002), SEQ ID No:18 (nm_{—}001880), SEQ ID No:121 (nm_{—}014553), SEQ ID No:1107 (BC073775), SEQ ID No:103 (nm_{—}003619), SEQ ID No:1118, SEQ ID No:42 (nm_{—}000757), and SEQ ID No:1067 (AK123784), fragments, derivatives or complementary sequences thereof;
c) generating a score (S_{C}) from said metagene adjusted values using a mathematical method establishing a relation between the combined metagene values and the clinical outcome of said female mammal.

98. The method of claim 97, wherein said metagene adjusted value underEGFR is generated by comparing the expression level, in a biological sample from said female mammal and in a control, of the nucleic acid sequence consisting of: SEQ ID No:681 (nm_{—}020974).

99. The method of claim 97, wherein said metagene adjusted value underEGFR is generated by comparing the expression level, in a biological sample from said female mammal and in a control, of the 24 nucleic acid sequences selected in the group consisting of: SEQ ID No:1071 (nm_{—}001033047); SEQ ID No:254 (nm_{—}005581); SEQ ID No:6 (nm_{—}003225); SEQ ID No:883 (nm_{—}000125); SEQ ID No:543 (nm_{—}005080); SEQ ID No:681 (nm_{—}020974); SEQ ID No:63 (nm_{—}001002295); SEQ ID No:212 (nm_{—}024852); SEQ ID No:635 (nm_{—}001002029); SEQ ID No:535 (nm_{—}003226); SEQ ID No:1125); SEQ ID No:1124; SEQ ID No:297 (nm_{—}016463); SEQ ID No:791 (nm_{—}016835); SEQ ID No:827 (nm_{—}152499); SEQ ID No:207 (nm_{—}003940); SEQ ID No:916 (nm_{—}001453) (or SEQ ID No:1116 (nm_{—}004040)); SEQ ID No:1052 (BX096026); SEQ ID No:159 (nm_{—}000224); SEQ ID No:25 (nm_{—}012391) (or SEQ ID No:1108 (NM_{—}053279)); SEQ ID No:845 (nm_{—}001089); and SEQ ID No:1085 (NM_{—}000720), fragments, derivatives or complementary sequences thereof.

100. The method of claim 97, wherein said metagene adjusted value underEGFR is generated by comparing the expression level, in a biological sample from said female mammal and in a control, of the 37 nucleic acid sequences selected in the group consisting of: SEQ ID No:1071 (nm_{—}001033047); SEQ ID No:254 (nm_{—}005581); SEQ ID No:6 (nm_{—}003225); SEQ ID No:883 (nm_{—}000125); SEQ ID No:543 (nm_{—}005080); SEQ ID No:681 (nm_{—}020974); SEQ ID No:63 (nm_{—}001002295); SEQ ID No:212 (nm_{—}024852); SEQ ID No:635 (nm_{—}001002029); SEQ ID No:535 (nm_{—}003226); SEQ ID No:1125; SEQ ID No:1124; SEQ ID No:297 (nm_{—}016463); SEQ ID No:791 (nm_{—}016835); SEQ ID No:827 (nm_{—}152499); SEQ ID No:207 (nm_{—}003940); SEQ ID No:916 (nm_{—}001453) (or SEQ ID No:1116 (nm_{—}004040)); SEQ ID No:1052 (BX096026); SEQ ID No:159 (nm_{—}000224); SEQ ID No:25 (nm_{—}012391) (or SEQ ID No:1108 (NM_{—}053279)); SEQ ID No:845 (nm_{—}001089); SEQ ID No:1085 (NM_{—}000720); SEQ ID No:109 (nm_{—}000662); SEQ ID No:342 (nm_{—}001846); SEQ ID No:927 (nm_{—}004703); SEQ ID No:280 (nm_{—}020764) (or SEQ ID No:1110 (NM_{—}024522)); SEQ ID No:210 (nm_{—}178840); SEQ ID No:181 (nm_{—}000848); SEQ ID No:116 (nm_{—}014034); SEQ ID No:250 (nm_{—}000930); SEQ ID No:177 (nm_{—}015996); SEQ ID No:825 (nm_{—}024704); SEQ ID No:145 (nm_{—}017786); and SEQ ID No:276 (nm_{—}012202), fragments, derivatives or complementary sequences thereof.

101. The method of claim 97, wherein the step b) of generating a metagene adjusted value overEGFR is obtained by comparing the expression level, in a biological sample from said female mammal and in a control, of at least 5 nucleic acid sequences selected in said group.

102. The method of claim 97, wherein said metagene adjusted value overEGFR is generated by comparing the expression level, in a biological sample from said female mammal and in a control, of the nucleic acid sequence consisting of: SEQ ID No: 1107 (BC073775) or SEQ ID No: 1099 (BC066343), fragments, derivatives or complementary sequences thereof.

103. The method of claim 97, wherein said metagene adjusted value overEGFR is generated by comparing the expression level, in a biological sample from said female mammal and in a control, of the 5 nucleic acid sequences selected in the group consisting of: SEQ ID No:1122; SEQ ID No:598 (nm_{—}000636); SEQ ID No:696 (nm_{—}001428); SEQ ID No:1059 (AK091113); and SEQ ID No:121 (nm_{—}014553), fragments, derivatives or complementary sequences thereof.

104. The method of claim 97, wherein said metagene adjusted value overEGFR is generated by comparing the expression level, in a biological sample from said female mammal and in a control, of the 12 nucleic acid sequences selected in the group consisting of: SEQ ID No:1122; SEQ ID No:598 (nm_{—}000636); SEQ ID No:696 (nm_{—}001428); SEQ ID No:1059 (AK091113); SEQ ID No:121 (nm_{—}014553); SEQ ID No:262 (nm_{—}005194); SEQ ID No:1099 (BC066343); SEQ ID No:751 (nm_{—}002423); SEQ ID No:1121; SEQ ID No:286 (nm_{—}002417); SEQ ID No:103 (nm_{—}003619); and SEQ ID No:1118, fragments, derivatives or complementary sequences thereof.

105. A method of assessing the clinical outcome of a female mammal suffering from breast cancer, comprising the steps of:
a) generating a metagene adjusted value underER by comparing the expression level, in a biological sample from said female mammal and in a control, of at least two genes, e.g. by using nucleic acid sequences selected in the group of Affymetrix® Probe Sets, of table IX or XII, preferably table XII,
b) generating said metagene adjusted value underPR by comparing the expression level, in a biological sample from said female mammal and in a control, of at least two genes, e.g. by using nucleic acid sequences selected in the group of Affymetrix® Probe Sets, of table X or XIII, preferably table XIII,
c) generating said metagene adjusted value underEGFR by comparing the expression level, in a biological sample from said female mammal and in a control, of at least two genes, e.g. by using nucleic acid sequences selected in the group of Affymetrix® Probe Sets, of table XI or XIV preferably table XIV,
d) generating a score (S_{C}) from said metagene adjusted values using a mathematical method establishing a relation between the combined metagene values and the clinical outcome of said female mammal.

106. The method of claim 90, 97 or 105, wherein the mathematical method used in step d) comprises a Cox regression or CART analysis.

107. The method of claim 90, 97 or 105, wherein the mathematical method used in step d) is a Cox regression and the score (S_{C}) is generated according to the following formula: S_{C}=a×underER+b×underPR+c×under EGFR, wherein “a” is comprised in the interval [−6.26; +0.49] “b” is comprised in the interval [−2.65; +0.29] and “c” is comprised in the interval [−6.69; +1.65].

108. The method of claim 90. 97 or 105, further comprising the step e) of comparing said score (S_{C}) from the biological sample with a baseline or a score (S_{C}) from a control sample.

109. The method of claim 90, 97 or 105, further comprising the step of administrating a pharmaceutical treatment to a female mammal, for optimizing the clinical outcome of said female mammal in response to said treatment.

110. The method of claim 90, 97 or 105, further comprising the step of generating a printed report.

111. A Computer program comprising instructions for performing the method according to claim 90, 97 or 105.

112. A recording medium for recording the computer program according to claim 110.
